SEQUENCE LISTING (221 sequences)

SEQ ID NO: 1 - protein, 12 aa, Artificial Sequence
  A5 peptide
HHHSPSYAYHQF

SEQ ID NO: 2 - protein, 6 aa, Artificial Sequence
  his-tag
HHHHHH

SEQ ID NO: 3 - protein, 5 aa, Artificial Sequence
  his-tag
HHHHH

SEQ ID NO: 4 - protein, 12 aa, Artificial Sequence
  single peptide vaccines
HHHYRDGNPYAV

SEQ ID NO: 5 - protein, 12 aa, Artificial Sequence
  single peptide vaccines
HHHFAFRDLCIV

SEQ ID NO: 6 - protein, 12 aa, Artificial Sequence
  single peptide vaccines
HHHKCLKFYSKI

SEQ ID NO: 7 - protein, 12 aa, Artificial Sequence
  single peptide vaccines
HHHTLHEYMLDL

SEQ ID NO: 8 - protein, 12 aa, Artificial Sequence
  single peptide vaccines
HHHRAHYNIVTF

SEQ ID NO: 9 - protein, 12 aa, Artificial Sequence
  single peptide vaccines
HHHSTHVDIRTL

SEQ ID NO: 10 - protein, 11 aa, Artificial Sequence
  Position-scanning libraries - WT
HHHSPHQVFNL

SEQ ID NO: 11 - protein, 11 aa, Artificial Sequence
  Position-scanning libraries Env37-44-Pos1
  MISC_FEATURE (4)..(4): X=A,R,N,D,C,Q,E,G,H,l,L,K,M,F,P,S,T,W,Y,V
HHHXPHQVFNL

SEQ ID NO: 12 - protein, 11 aa, Artificial Sequence
  Env37-44-Pos2
  MISC_FEATURE (5)..(5): X=A,R,N,D,C,Q,E,G,H,l,L,K,M,F,P,S,T,W,Y,V
HHHSXHQVFNL

SEQ ID NO: 13 - protein, 11 aa, Artificial Sequence
  Env37-44-Pos3
  MISC_FEATURE (6)..(6): X=A,R,N,D,C,Q,E,G,H,l,L,K,M,F,P,S,T,W,Y,V
HHHSPXQVFNL

SEQ ID NO: 14 - protein, 11 aa, Artificial Sequence
  Env37-44-Pos4
  MISC_FEATURE (7)..(7): X=A,R,N,D,C,Q,E,G,H,l,L,K,M,F,P,S,T,W,Y,V
HHHSPHXVFNL

SEQ ID NO: 15 - protein, 11 aa, Artificial Sequence
  Env37-44-Pos5
  MISC_FEATURE (8)..(8): X=A,R,N,D,C,Q,E,G,H,l,L,K,M,F,P,S,T,W,Y,V
HHHSPHQXFNL

SEQ ID NO: 16 - protein, 11 aa, Artificial Sequence
  Env37-44-Pos6
  MISC_FEATURE (9)..(9): X=A,R,N,D,C,Q,E,G,H,l,L,K,M,F,P,S,T,W,Y,V
HHHSPHQVXNL

SEQ ID NO: 17 - protein, 11 aa, Artificial Sequence
  Env37-44-Pos7
  MISC_FEATURE (10)..(10): X=A,R,N,D,C,Q,E,G,H,l,L,K,M,F,P,S,T,W,Y,V
HHHSPHQVFXL

SEQ ID NO: 18 - protein, 11 aa, Artificial Sequence
  Env37-44-Pos8
  MISC_FEATURE (11)..(11): X=A,R,N,D,C,Q,E,G,H,l,L,K,M,F,P,S,T,W,Y,V
HHHSPHQVFNX

SEQ ID NO: 19 - protein, 12 aa, Artificial Sequence
  AH1 positional peptide libraries WT
HHHSPSYVYHQF

SEQ ID NO: 20 - protein, 12 aa, Artificial Sequence
  AH1-Pos1
  MISC_FEATURE (4)..(4): X=A,R,N,D,C,Q,E,G,H,l,L,K,M,F,P,S,T,W,Y,V
HHHXPSYVYHQF

SEQ ID NO: 21 - protein, 12 aa, Artificial Sequence
  AH1-Pos2
  MISC_FEATURE (5)..(5): X=A,R,N,D,C,Q,E,G,H,l,L,K,M,F,P,S,T,W,Y,V
HHHSXSYVYHQF

SEQ ID NO: 22 - protein, 12 aa, Artificial Sequence
  AH1-Pos3
  MISC_FEATURE (6)..(6): X=A,R,N,D,C,Q,E,G,H,l,L,K,M,F,P,S,T,W,Y,V
HHHSPXYVYHQF

SEQ ID NO: 23 - protein, 12 aa, Artificial Sequence
  AH1-Pos4
  MISC_FEATURE (7)..(7): X=A,R,N,D,C,Q,E,G,H,l,L,K,M,F,P,S,T,W,Y,V
HHHSPSXVYHQF

SEQ ID NO: 24 - protein, 12 aa, Artificial Sequence
  AH1-Pos5
  MISC_FEATURE (8)..(8): X=A,R,N,D,C,Q,E,G,H,l,L,K,M,F,P,S,T,W,Y,V
HHHSPSYXYHQF

SEQ ID NO: 25 - protein, 12 aa, Artificial Sequence
  AH1-Pos6
  MISC_FEATURE (9)..(9): X=A,R,N,D,C,Q,E,G,H,l,L,K,M,F,P,S,T,W,Y,V
HHHSPSYVXHQF

SEQ ID NO: 26 - protein, 12 aa, Artificial Sequence
  AH1-Pos7
  MISC_FEATURE (10)..(10): X=A,R,N,D,C,Q,E,G,H,l,L,K,M,F,P,S,T,W,Y,V
HHHSPSYVYXQF

SEQ ID NO: 27 - protein, 12 aa, Artificial Sequence
  AH1-Pos8
  MISC_FEATURE (11)..(11): X=A,R,N,D,C,Q,E,G,H,l,L,K,M,F,P,S,T,W,Y,V
HHHSPSYVYHXF

SEQ ID NO: 28 - protein, 12 aa, Artificial Sequence
  AH1-Pos9
  MISC_FEATURE (12)..(12): X=A,R,N,D,C,Q,E,G,H,l,L,K,M,F,P,S,T,W,Y,V
HHHSPSYVYHQX

SEQ ID NO: 29 - protein, 10 aa, Artificial Sequence
  Survivin T97M
ELMLGEFLKL

SEQ ID NO: 30 - protein, 10 aa, Artificial Sequence
  Melan A/MART126-35
EAAGIGILTV

SEQ ID NO: 31 - protein, 9 aa, Artificial Sequence
  Melan A/MART127-35
AAGIGILTV

SEQ ID NO: 32 - protein, 9 aa, Artificial Sequence
  Tyrosinase1-9
MLLAVLYCL

SEQ ID NO: 33 - protein, 9 aa, Artificial Sequence
  Tyrosinase368-376
YMDGTMSQV

SEQ ID NO: 34 - protein, 10 aa, Artificial Sequence
  Gp100457-466
LLDGTATLRL

SEQ ID NO: 35 - protein, 9 aa, Artificial Sequence
  Survivin-2B80-88
AYACNTSTL

SEQ ID NO: 36 - protein, 9 aa, Artificial Sequence
  NY-ESO-1b157-165
SLLMWITQC

SEQ ID NO: 37 - protein, 9 aa, Artificial Sequence
  WT1235-243
CMTWNQMNL

SEQ ID NO: 38 - protein, 9 aa, Artificial Sequence
  gp100209-217 (210M)
IMDQVPFSV

SEQ ID NO: 39 - protein, 9 aa, Artificial Sequence
  gp100280-288 (288V)
YLEPGPVTA

SEQ ID NO: 40 - protein, 10 aa, Artificial Sequence
  E711-20
YMLDLQPETT

SEQ ID NO: 41 - protein, 8 aa, Artificial Sequence
  E786-93
TLGIVCPI

SEQ ID NO: 42 - protein, 9 aa, Artificial Sequence
  E782-90
LLMGTLGIV

SEQ ID NO: 43 - protein, 9 aa, Artificial Sequence
  functional neo-antigen
AYTTQREEL

SEQ ID NO: 44 - protein, 9 aa, Artificial Sequence
  RragcL385P peptide
SPKALAHNG

SEQ ID NO: 45 - protein, 9 aa, Artificial Sequence
  Trp2180-188
SVYDFFVWL

SEQ ID NO: 46 - DNA, 20 bp, Artificial Sequence
  forward primer
tcactgttcacgtctgtcct

SEQ ID NO: 47 - DNA, 19 bp, Artificial Sequence
  reverse primer
actgagttctgaggtctct

SEQ ID NO: 48 - protein, 18 aa, Artificial Sequence
  PEP1
HHHHHHSPSYAYHQFERR

SEQ ID NO: 49 - protein, 18 aa, Artificial Sequence
  PEP2
HHHHHHSPSYAYHQFEEE

SEQ ID NO: 50 - protein, 18 aa, Artificial Sequence
  PEP3
HHHHHHSPSYAYHQFRRR

SEQ ID NO: 51 - protein, 15 aa, Artificial Sequence
  PEP4
HHHHHHSPSYAYHQF

SEQ ID NO: 52 - protein, 9 aa, Artificial Sequence
  PEP5
SPSYAYHQF

SEQ ID NO: 53 - protein, 12 aa, Artificial Sequence
  PEP6
SPSYAYHQFERR

SEQ ID NO: 54 - protein, 13 aa, Artificial Sequence
  PEP7
HSPSYAYHQFERR

SEQ ID NO: 55 - protein, 14 aa, Artificial Sequence
  PEP8
HHSPSYAYHQFERR

SEQ ID NO: 56 - protein, 15 aa, Artificial Sequence
  PEP9
HHHSPSYAYHQFERR

SEQ ID NO: 57 - protein, 16 aa, Artificial Sequence
  PEP10
HHHHSPSYAYHQFERR

SEQ ID NO: 58 - protein, 17 aa, Artificial Sequence
  PEP11
HHHHHSPSYAYHQFERR

SEQ ID NO: 59 - protein, 12 aa, Artificial Sequence
  PEP12
HHHSPSYVYHQF

SEQ ID NO: 60 - protein, 19 aa, Artificial Sequence
  PEP13
  MISC_FEATURE (19)..(19): Functionalized with a fluorophore
HHHHHHSPSYAYHQFERRX

SEQ ID NO: 61 - protein, 15 aa, Artificial Sequence
  PEP14
  MISC_FEATURE (15)..(15): Functionalized with a fluorophore
HHSPSYAYHQFERRX

SEQ ID NO: 62 - protein, 18 aa, Artificial Sequence
  CT1
HHHHHHSGPSYATYLERR

SEQ ID NO: 63 - protein, 18 aa, Artificial Sequence
  CT2
HHHHHHKGPKRDEQCERR

SEQ ID NO: 64 - protein, 18 aa, Artificial Sequence
  CT3
HHHHHHPGPIKCVPIERR

SEQ ID NO: 65 - protein, 18 aa, Artificial Sequence
  CT4
HHHHHHGPPGGFQEFERR

SEQ ID NO: 66 - protein, 18 aa, Artificial Sequence
  CT5
HHHHHHQFPSESEFFERR

SEQ ID NO: 67 - protein, 18 aa, Artificial Sequence
  CT6
HHHHHHTGPYVMMIGERR

SEQ ID NO: 68 - protein, 18 aa, Artificial Sequence
  CT7
HHHHHHSYETLKKSLERR

SEQ ID NO: 69 - protein, 18 aa, Artificial Sequence
  CT8
HHHHHHKYYAVNFSMERR

SEQ ID NO: 70 - protein, 18 aa, Artificial Sequence
  CT9
HHHHHHCYVTYKGEIERR

SEQ ID NO: 71 - protein, 18 aa, Artificial Sequence
  CT10
HHHHHHSYTSYIMAIERR

SEQ ID NO: 72 - protein, 18 aa, Artificial Sequence
  CT11
HHHHHHRYWTGIGVLERR

SEQ ID NO: 73 - protein, 18 aa, Artificial Sequence
  CT12
HHHHHHAYVNAIEKIERR

SEQ ID NO: 74 - protein, 18 aa, Artificial Sequence
  CT13
HHHHHHAYVNATGEVERR

SEQ ID NO: 75 - protein, 18 aa, Artificial Sequence
  CT14
HHHHHHFPPQNMFEFERR

SEQ ID NO: 76 - protein, 18 aa, Artificial Sequence
  CT15
HHHHHHEPQVEPLDFERR

SEQ ID NO: 77 - protein, 18 aa, Artificial Sequence
  CT16
HHHHHHIPDEAMVKLERR

SEQ ID NO: 78 - protein, 18 aa, Artificial Sequence
  CT17
HHHHHHSPNRRFTEMERR

SEQ ID NO: 79 - protein, 18 aa, Artificial Sequence
  CT18
HHHHHHRPIKGPRNIERR

SEQ ID NO: 80 - protein, 18 aa, Artificial Sequence
  CT19
HHHHHHVVLETFYDLERR

SEQ ID NO: 81 - protein, 18 aa, Artificial Sequence
  CT20
HHHHHHNPRAMQVLLERR

SEQ ID NO: 82 - protein, 18 aa, Artificial Sequence
  CT21
HHHHHHDPFATPLSMERR

SEQ ID NO: 83 - protein, 18 aa, Artificial Sequence
  CT22
HHHHHHEPQIAMDDMERR

SEQ ID NO: 84 - protein, 18 aa, Artificial Sequence
  CT23
HHHHHHGYEAVVRLLERR

SEQ ID NO: 85 - protein, 18 aa, Artificial Sequence
  CT24
HHHHHHHHTMMVQAIERR

SEQ ID NO: 86 - protein, 18 aa, Artificial Sequence
  CT25
HHHHHHHYLNDGDAIERR

SEQ ID NO: 87 - protein, 18 aa, Artificial Sequence
  CT26
HHHHHHLFVTVLEVIERR

SEQ ID NO: 88 - protein, 18 aa, Artificial Sequence
  CT27
HHHHHHPYFRLEHYLERR

SEQ ID NO: 89 - protein, 18 aa, Artificial Sequence
  CT28
HHHHHHPYLTALDDLERR

SEQ ID NO: 90 - protein, 18 aa, Artificial Sequence
  CT29
HHHHHHSYIKKLKELERR

SEQ ID NO: 91 - protein, 18 aa, Artificial Sequence
  CT30
HHHHHHSYTEQNVKLERR

SEQ ID NO: 92 - protein, 18 aa, Artificial Sequence
  CT31
HHHHHHTYWKGNPEMERR

SEQ ID NO: 93 - protein, 18 aa, Artificial Sequence
  CT32
HHHHHHVYFTINVNLERR

SEQ ID NO: 94 - protein, 18 aa, Artificial Sequence
  CT33
HHHHHHYYMRDVIAIERR

SEQ ID NO: 95 - protein, 18 aa, Artificial Sequence
  CT34
HHHHHHLFLKSPTALERR

SEQ ID NO: 96 - protein, 18 aa, Artificial Sequence
  CT35
HHHHHHSYLRRLPGLERR

SEQ ID NO: 97 - protein, 18 aa, Artificial Sequence
  CT36*
HHHHHHKTPELVEALERR

SEQ ID NO: 98 - protein, 18 aa, Artificial Sequence
  CT37*
HHHHHHSPKALAHNGERR

SEQ ID NO: 99 - protein, 18 aa, Artificial Sequence
  CT38*
HHHHHHSSKHRRSFVERR

SEQ ID NO: 100 - protein, 18 aa, Artificial Sequence
  CT39*
HHHHHHKLLASALSTERR

SEQ ID NO: 101 - protein, 18 aa, Artificial Sequence
  CT40*
HHHHHHNALSSMGALERR

SEQ ID NO: 102 - protein, 18 aa, Artificial Sequence
  CT41
HHHHHHRTPHVLWSWERR

SEQ ID NO: 103 - protein, 18 aa, Artificial Sequence
  CT42
HHHHHHWGAWLSGALERR

SEQ ID NO: 104 - protein, 18 aa, Artificial Sequence
  CT43
HHHHHHFPPQNMFEFERR

SEQ ID NO: 105 - protein, 18 aa, Artificial Sequence
  CT44
HHHHHHKGPYWLEVKERR

SEQ ID NO: 106 - protein, 18 aa, Artificial Sequence
  CT45
HHHHHHLVPNRFSRLERR

SEQ ID NO: 107 - protein, 18 aa, Artificial Sequence
  CT46
HHHHHHRGPPPHSAPERR

SEQ ID NO: 108 - protein, 18 aa, Artificial Sequence
  CT47
HHHHHHGPPPHSAPCERR

SEQ ID NO: 109 - protein, 18 aa, Artificial Sequence
  CT48
HHHHHHSGPIGPPEIERR

SEQ ID NO: 110 - protein, 18 aa, Artificial Sequence
  CT49
HHHHHHTPPSIFSTQERR

SEQ ID NO: 111 - protein, 18 aa, Artificial Sequence
  CT50
HHHHHHYGKGVNFYLERR

SEQ ID NO: 112 - protein, 18 aa, Artificial Sequence
  CT51
HHHHHHGRPSIDNVLERR

SEQ ID NO: 113 - protein, 18 aa, Artificial Sequence
  CT52
HHHHHHPTPRRFSPPERR

SEQ ID NO: 114 - protein, 18 aa, Artificial Sequence
  CT53
HHHHHHRGGPKKCALERR

SEQ ID NO: 115 - protein, 18 aa, Artificial Sequence
  CT54
HHHHHHEGPHGQPVSERR

SEQ ID NO: 116 - protein, 18 aa, Artificial Sequence
  CT55
HHHHHHTPPSHKLPLERR

SEQ ID NO: 117 - protein, 18 aa, Artificial Sequence
  CT56
HHHHHHRGEYEDPYYERR

SEQ ID NO: 118 - protein, 18 aa, Artificial Sequence
  CT57
HHHHHHKGPRNIQLDERR

SEQ ID NO: 119 - protein, 18 aa, Artificial Sequence
  CT58
HHHHHHGFPSPLGGIERR

SEQ ID NO: 120 - protein, 18 aa, Artificial Sequence
  CT59
HHHHHHTGRRRTATKERR

SEQ ID NO: 121 - protein, 18 aa, Artificial Sequence
  CT60
HHHHHHDSPNRRFTEERR

SEQ ID NO: 122 - protein, 18 aa, Artificial Sequence
  CT61
HHHHHHCQIAMVHYIERR

SEQ ID NO: 123 - protein, 18 aa, Artificial Sequence
  CT62
HHHHHHSFQPTADELERR

SEQ ID NO: 124 - protein, 18 aa, Artificial Sequence
  CT63
HHHHHHSYATYLQPAERR

SEQ ID NO: 125 - protein, 18 aa, Artificial Sequence
  CT64
HHHHHHLYLANLRHMERR

SEQ ID NO: 126 - protein, 18 aa, Artificial Sequence
  CT65
HHHHHHYYESDEFTVERR

SEQ ID NO: 127 - protein, 18 aa, Artificial Sequence
  CT66
HHHHHHRHNTMLKAMERR

SEQ ID NO: 128 - protein, 18 aa, Artificial Sequence
  CT67
HHHHHHRHNTMLKAMERR

SEQ ID NO: 129 - protein, 18 aa, Artificial Sequence
  CT68
HHHHHHEYEKIEIPIERR

SEQ ID NO: 130 - protein, 18 aa, Artificial Sequence
  CT69
HHHHHHKYFQAGKYEERR

SEQ ID NO: 131 - protein, 18 aa, Artificial Sequence
  CT70
HHHHHHTYAIALINVERR

SEQ ID NO: 132 - protein, 18 aa, Artificial Sequence
  CT71
HHHHHHVYFVAGVRVERR

SEQ ID NO: 133 - protein, 18 aa, Artificial Sequence
  CT72
HHHHHHSRLQSLTLIERR

SEQ ID NO: 134 - protein, 18 aa, Artificial Sequence
  CT73
HHHHHHFFMKMASKFERR

SEQ ID NO: 135 - protein, 18 aa, Artificial Sequence
  CT74
HHHHHHFYTSYLRRLERR

SEQ ID NO: 136 - protein, 18 aa, Artificial Sequence
  CT75
HHHHHHTYLQPAQAQERR

SEQ ID NO: 137 - protein, 18 aa, Artificial Sequence
  CT76
HHHHHHIYSRTTDVLERR

SEQ ID NO: 138 - protein, 18 aa, Artificial Sequence
  CT77
HHHHHHSYQAALAENERR

SEQ ID NO: 139 - protein, 18 aa, Artificial Sequence
  CT78
HHHHHHIYLESVAIMERR

SEQ ID NO: 140 - protein, 18 aa, Artificial Sequence
  CT79
HHHHHHGHVAAGGALERR

SEQ ID NO: 141 - protein, 18 aa, Artificial Sequence
  CT81
HHHHHHSEGNSSHTIERR

SEQ ID NO: 142 - protein, 18 aa, Artificial Sequence
  CT81
HHHHHHLNYPHITNLERR

SEQ ID NO: 143 - protein, 18 aa, Artificial Sequence
  CT82
HHHHHHPPQNMFEFPERR

SEQ ID NO: 144 - protein, 18 aa, Artificial Sequence
  CT83
HHHHHHFPSESEFFKERR

SEQ ID NO: 145 - protein, 18 aa, Artificial Sequence
  CT84
HHHHHHIGQMLQTHFERR

SEQ ID NO: 146 - protein, 18 aa, Artificial Sequence
  CT85
HHHHHHIPIFHILNTERR

SEQ ID NO: 147 - protein, 18 aa, Artificial Sequence
  CT86
HHHHHHMLARKQFDLERR

SEQ ID NO: 148 - protein, 18 aa, Artificial Sequence
  CT87
HHHHHHKPYESYTEQERR

SEQ ID NO: 149 - protein, 18 aa, Artificial Sequence
  CT88
HHHHHHKPFTICKYYERR

SEQ ID NO: 150 - protein, 18 aa, Artificial Sequence
  CT89
HHHHHHPGGFQEFNFERR

SEQ ID NO: 151 - protein, 18 aa, Artificial Sequence
  CT90
HHHHHHTALPVGFSLERR

SEQ ID NO: 152 - protein, 18 aa, Artificial Sequence
  CT91
HHHHHHRKIKGPYWLERR

SEQ ID NO: 153 - protein, 18 aa, Artificial Sequence
  CT92
HHHHHHLGYRYWTGIERR

SEQ ID NO: 154 - protein, 18 aa, Artificial Sequence
  CT93
HHHHHHLLYNAVVWLERR

SEQ ID NO: 155 - protein, 18 aa, Artificial Sequence
  CT94
HHHHHHDPQFAENYVERR

SEQ ID NO: 156 - protein, 18 aa, Artificial Sequence
  CT95
HHHHHHKYYAVNFSMERR

SEQ ID NO: 157 - protein, 18 aa, Artificial Sequence
  CT96
HHHHHHVVEANWTMLERR

SEQ ID NO: 158 - protein, 18 aa, Artificial Sequence
  CT97
HHHHHHTPHVLWSWGERR

SEQ ID NO: 159 - protein, 18 aa, Artificial Sequence
  CT98
HHHHHHYVYEIYMTFERR

SEQ ID NO: 160 - protein, 18 aa, Artificial Sequence
  CT99
HHHHHHGPPGGFQEFERR

SEQ ID NO: 161 - protein, 18 aa, Artificial Sequence
  CT100
HHHHHHMPQLFMVSKERR

SEQ ID NO: 162 - protein, 12 aa, Artificial Sequence
  3his-CT37/RragcL385P
HHHSPKALAHNG

SEQ ID NO: 163 - protein, 12 aa, Artificial Sequence
  3his- CT71/Eml5G44R
HHHVYFVAGVRV

SEQ ID NO: 164 - protein, 12 aa, Artificial Sequence
  3his-CT96/Tmem5S71N
HHHVVEANWTML

SEQ ID NO: 165 - protein, 12 aa, Artificial Sequence
  Renca_1
HHHAYATTYRQL

SEQ ID NO: 166 - protein, 12 aa, Artificial Sequence
  Renca_2
HHHAYTTQREEL

SEQ ID NO: 167 - protein, 12 aa, Artificial Sequence
  Renca_3
HHHEGARRMFTW

SEQ ID NO: 168 - protein, 12 aa, Artificial Sequence
  Renca_4
HHHFPAVVQCTL

SEQ ID NO: 169 - protein, 12 aa, Artificial Sequence
  Renca_5
HHHGGTSRGKLW

SEQ ID NO: 170 - protein, 12 aa, Artificial Sequence
  Renca_6
HHHGLPYWVTIL

SEQ ID NO: 171 - protein, 12 aa, Artificial Sequence
  Renca_6
HHHGYFGAVGAI

SEQ ID NO: 172 - protein, 12 aa, Artificial Sequence
  Renca_8
HHHGYHESGHAI

SEQ ID NO: 173 - protein, 12 aa, Artificial Sequence
  Renca_9
HHHHGPQLECTV

SEQ ID NO: 174 - protein, 12 aa, Artificial Sequence
  Renca_10
HHHIHETTFTSI

SEQ ID NO: 175 - protein, 12 aa, Artificial Sequence
  Renca_11
HHHIYVLARTRL

SEQ ID NO: 176 - protein, 12 aa, Artificial Sequence
  Renca_12
HHHLYAVATTLV

SEQ ID NO: 177 - protein, 12 aa, Artificial Sequence
  Renca_13
HHHMPFANSPWV

SEQ ID NO: 178 - protein, 12 aa, Artificial Sequence
  Renca_14
HHHSYLQGHTQQ

SEQ ID NO: 179 - protein, 12 aa, Artificial Sequence
  Renca_15
HHHSYVMISRAL

SEQ ID NO: 180 - protein, 12 aa, Artificial Sequence
  Renca_16
HHHTYFTSATMI

SEQ ID NO: 181 - protein, 12 aa, Artificial Sequence
  Renca_17
HHHVGPGALAVL

SEQ ID NO: 182 - protein, 12 aa, Artificial Sequence
  Renca_18
HHHVPNTCLFTI

SEQ ID NO: 183 - protein, 12 aa, Artificial Sequence
  Renca_19
HHHVPSVQTFLM

SEQ ID NO: 184 - protein, 12 aa, Artificial Sequence
  Renca_20
HHHYYLDVGKTL

SEQ ID NO: 185 - protein, 12 aa, Artificial Sequence
  E749-57
HHHRAHYNIVTF

SEQ ID NO: 186 - protein, 12 aa, Artificial Sequence
  E771-79
HHHSTHVDIRTL

SEQ ID NO: 187 - protein, 12 aa, Artificial Sequence
  E76-14
HHHTLHEYMLDL

SEQ ID NO: 188 - protein, 12 aa, Artificial Sequence
  E661-69
HHHYRDGNPYAV

SEQ ID NO: 189 - protein, 12 aa, Artificial Sequence
  E662-70
HHHFAFRDLCIV

SEQ ID NO: 190 - protein, 12 aa, Artificial Sequence
  E682-90
HHHKCLKFYSKI

SEQ ID NO: 191 - protein, 12 aa, Artificial Sequence
  Trp2_1
  misc_feature (4)..(4): Xaa can be any naturally occurring amino acid
HHHXVYDFFVWL

SEQ ID NO: 192 - protein, 12 aa, Artificial Sequence
  Trp2_2
  misc_feature (5)..(5): Xaa can be any naturally occurring amino acid
HHHSXYDFFVWL

SEQ ID NO: 193 - protein, 12 aa, Artificial Sequence
  Trp2_3
  misc_feature (6)..(6): Xaa can be any naturally occurring amino acid
HHHSVXDFFVWL

SEQ ID NO: 194 - protein, 12 aa, Artificial Sequence
  Trp2_4
  misc_feature (7)..(7): Xaa can be any naturally occurring amino acid
HHHSVYXFFVWL

SEQ ID NO: 195 - protein, 12 aa, Artificial Sequence
  Trp2_5
  misc_feature (8)..(8): Xaa can be any naturally occurring amino acid
HHHSVYDXFVWL

SEQ ID NO: 196 - protein, 12 aa, Artificial Sequence
  Trp2_6
  misc_feature (9)..(9): Xaa can be any naturally occurring amino acid
HHHSVYDFXVWL

SEQ ID NO: 197 - protein, 12 aa, Artificial Sequence
  Trp2_7
  misc_feature (10)..(10): Xaa can be any naturally occurring amino acid
HHHSVYDFFXWL

SEQ ID NO: 198 - protein, 12 aa, Artificial Sequence
  Trp2_8
  misc_feature (11)..(11): Xaa can be any naturally occurring amino acid
HHHSVYDFFVXL

SEQ ID NO: 199 - protein, 12 aa, Artificial Sequence
  Trp2_9
  misc_feature (12)..(12): Xaa can be any naturally occurring amino acid
HHHSVYDFFVWX

SEQ ID NO: 200 - protein, 12 aa, Artificial Sequence
  HHH-Trp2
  misc_feature (12)..(12): Xaa can be any naturally occurring amino acid
HHHSVYDFFVWX

SEQ ID NO: 201 - protein, 12 aa, Artificial Sequence
  HHH-Trp2_8A
HHHSVYDFFVAL

SEQ ID NO: 202 - protein, 12 aa, Artificial Sequence
  HHH-Trp2_8R
HHHSVYDFFVRL

SEQ ID NO: 203 - protein, 12 aa, Artificial Sequence
  HHH-Trp2_8N
HHHSVYDFFVNL

SEQ ID NO: 204 - protein, 12 aa, Artificial Sequence
  HHH-Trp2_8D
HHHSVYDFFVDL

SEQ ID NO: 205 - protein, 12 aa, Artificial Sequence
  HHH-Trp2_8C
HHHSVYDFFVCL

SEQ ID NO: 206 - protein, 12 aa, Artificial Sequence
  HHH-Trp2_8Q
HHHSVYDFFVQL

SEQ ID NO: 207 - protein, 12 aa, Artificial Sequence
  HHH-Trp2_8E
HHHSVYDFFVEL

SEQ ID NO: 208 - protein, 12 aa, Artificial Sequence
  HHH-Trp2_8G
HHHSVYDFFVGL

SEQ ID NO: 209 - protein, 12 aa, Artificial Sequence
  HHH-Trp2_8H
HHHSVYDFFVHL

SEQ ID NO: 210 - protein, 12 aa, Artificial Sequence
  HHH-Trp2_8I
HHHSVYDFFVIL

SEQ ID NO: 211 - protein, 12 aa, Artificial Sequence
  HHH-Trp2_8L
HHHSVYDFFVLL

SEQ ID NO: 212 - protein, 12 aa, Artificial Sequence
  HHH-Trp2_8K
HHHSVYDFFVKL

SEQ ID NO: 213 - protein, 12 aa, Artificial Sequence
  HHH-Trp2_8M
HHHSVYDFFVML

SEQ ID NO: 214 - protein, 12 aa, Artificial Sequence
  HHH-Trp2_8F
HHHSVYDFFVFL

SEQ ID NO: 215 - protein, 12 aa, Artificial Sequence
  HHH-Trp2_8P
HHHSVYDFFVPL

SEQ ID NO: 216 - protein, 12 aa, Artificial Sequence
  HHH-Trp2_8S
HHHSVYDFFVSL

SEQ ID NO: 217 - protein, 12 aa, Artificial Sequence
  HHH-Trp2_8T
HHHSVYDFFVTL

SEQ ID NO: 218 - protein, 12 aa, Artificial Sequence
  HHH-Trp2_8W
HHHSVYDFFVWL

SEQ ID NO: 219 - protein, 12 aa, Artificial Sequence
  HHH-Trp2_8Y
HHHSVYDFFVYL

SEQ ID NO: 220 - protein, 12 aa, Artificial Sequence
  HHH-Trp2_8V
HHHSVYDFFVVL

SEQ ID NO: 221 - protein, 9 aa, Artificial Sequence
  Trp2
SVYDFFVWL

(19) United States
(12) Patent Application Publication
(10) Pub. No.: US 2024/0091328 A1
(43) Pub. Date: Mar. 21, 2024

(54) FORMULATIONS AND METHODS FOR MHC-I RESTRICTED EPITOPE IMMUNIZATION

(71) Applicant: The Research Foundation for The State University of New York, Amherst, NY (US)

(72) Inventors: Jonathan LOVELL, Niagara Falls (CA); Xuedan HE, Amherst, NY (US); Shiqi ZHOU, Amherst, NY (US)

(21) Appl. No.: 18/261,456

(22) PCT Filed: Jan. 11, 2022
(86) PCT No.: PCT/US2022/012045

§ 371 Date: Jul. 13, 2023

Related U.S. Application Data
(60) Provisional application No. 63/137,036, filed on Jan. 13, 2021.

(51) Int. Cl. A61K 39/00; A61K 9/127; A61P 35/00; C07K 7/06

(52) U.S. Cl. CPC A61K 39/001102; A61K 9/1271; A61P 35/00; C07K 7/06; A61K 2039/55555; A61K 2039/55572; A61K 2039/55577

(57) ABSTRACT

The present disclosure provides compositions and methods for generating immune response or enhancing immune response. The method comprises administering to a subject in need of treatment a compositions comprising liposomes comprising porphyrins with cobalt chelated thereto such that the cobalt metal resides within the bilayer in the porphyrin macrocycle, and polyhistidine tagged MHC-I restricted tumor peptides.

### CROSS-REFERENCE TO RELATED APPLICATIONS

This application claims priority to U.S. provisional patent application No. 63/137,036, filed on Jan. 13, 2021, the disclosure of which is incorporated herein by reference.

### STATEMENT REGARDING FEDERALLY SPONSORED RESEARCH

This invention was made with government support under grant number R01 CA247771 awarded by the National Institutes of Health. The government has certain rights in the invention.

### BACKGROUND OF THE DISCLOSURE

To kill cancer cells, the CD8⁺ T cell receptor (TCR) must recognize short tumor-derived peptides of 8-10 amino acids in association with major histocompatibility complex (MHC) class I (MHC-I) molecules. These short peptide epitopes are appealing for cancer vaccine development as they are simple to produce and provide, in theory, a direct method to induce CD8⁺ T cells against the antigen (Ag)-bearing target cell. Unfortunately, peptide-based vaccine cancer clinical trials have not produced compelling clinical responses in contrast to more recent immunotherapies, such as immune checkpoint blockade. While a number of reasons may account for this, one challenge for peptide-based cancer vaccines is the inability to potently generate Ag-specific CD8⁺ T cells with sufficient quantity and quality. Considerable efforts have been devoted to the development of improved cancer peptide vaccine systems. Covalent conjugation is often employed to improve their immunogenicity and to this end, recent promising approaches have exploited conjugation to lipids (Kuai et al., Nature materials 16, 489-496 (2017), proteins (Mehta et al., Nature Biomedical Engineering, doi:10.1038/s41551-020-0563-4 (2020)), and long peptides (Lynn, et al. Nature Biotechnology, 1-13 (2020)).

Another challenge for cancer vaccine development is to identify those short peptide immunogens that induce Ag-specific CD8⁺ T cell responses capable of recognizing the target epitope on cancer cells. Neoantigens are mutated cancer-specific epitopes that provide a rich source of potential cancer vaccine targets. However, identifying immunogenic MHC-I-restricted neoantigens that give rise to functional immune responses has proven difficult. Indeed, attempts to target these using long peptides led to the finding that the long peptides exert efficacy through MHC class II (MHC-II) binding and CD4⁺ (not CD8⁺) T cell help (Kreiter, et al. Nature 520, 692-696, doi:10.1038/nature14426 (2015)). This complicates design of MHC-I and the practical implication is that the design of short peptides (which, by virtue of their length, are restricted to binding MHC class I) is an emerging practice, with limited capacity for rational selection of target amino acid sequences.

### SUMMARY OF THE DISCLOSURE

The present disclosure provides compositions and methods for generating an anti-tumor immune response or enhancing an immune response to MIC-I peptides using functionalized liposomes comprising MHC-I peptides.

In an aspect, the present disclosure provides functionalized liposomes (also referred to herein as nanostructures) comprising MIC-I binding peptides (also referred to herein as MIC-I restricted peptides and MHC-I targeting peptides). While reference to MIC-I is used in this disclosure, the disclosure includes peptides that bind to human leukocyte antigen (HLA) Class 1 molecules. Thus, where reference is made to MHC-I, the disclosure includes HLA-1 restricted peptides for use in humans. It is considered that any peptide described herein that is demonstrated or predicted to bind MHC-I and/or stimulate CD8+ T cells will have the same properties if used HLA-1 expressing T Cells. In embodiments, it is considered that the described peptides that can bind to MHC- and stimulate CD8+ T cells are presented in an MHC-I context.

For example, the liposomes can comprise human MHC-I binding peptides. The bilayer comprises cobalt porphyrin-phospholipid conjugate, phospholipids that are not conjugated to porphyrin, optionally, sterols and optionally polyethylene glycol (PEG). One or more MHC-I binding peptides having a polyhistidine tag are incorporated into the bilayer such that a portion of the polyhistidine tag resides in the bilayer and at least a portion of the MIC-I targeting peptide is exposed to the exterior of the bilayer. Instead of, or in addition to the cobalt porphyrin phospholipid conjugate, cobalt porphyrin can be used in the liposomal bilayers. The bilayer structures comprise porphyrins with cobalt chelated thereto such that the cobalt metal resides within the bilayer and the porphyrin macrocycle and further have MIC-I binding peptide molecules with a histidine tag non-covalently attached thereto, such that at least a part of the his-tag is within the bilayer and coordinated to the cobalt metal core. In embodiments, the cobalt porphyrin is cobalt porphyrin-phospholipid (CoPoP). The present liposomes may further comprise adjuvants incorporated in the bilayer or residing in the aqueous compartment or both. For example, in embodiments the liposomes further comprise QS21 and PHAD. CPQ refers to liposomes that include CoPoP, a PHAD variant and QS21. 2HPQ refers to liposomes that are identical but lack the cobalt in porphyrin macrocycle, and so they contain PoP, a PHAD variant and QS21.

In various embodiments, the present disclosure provides a liposome comprising: a) a bilayer, wherein the bilayer comprises: i) phospholipid, and ii) porphyrin having cobalt coordinated thereto forming cobalt-porphyrin; and b) a polyhistidine-tagged MHC-I restricted peptide, wherein at least a portion of the polyhistidine tag resides in the hydrophobic portion of the bilayer and one or more histidines of the polyhistidine tag are coordinated to the cobalt in the cobalt-porphyrin, wherein at least a portion of the polyhistidine-tagged MHC-I restricted peptide is exposed to the outside of the liposome, and the liposome binds to MHC-I class of molecules, but not MHC-II class of molecules, the polyhistidine-tag comprises 2-6 histidine residues (preferably less than 6 histidine residues), and the MHC-I restricted peptide is a peptide from 4 to 11 amino acids in length, including all integer values and ranges therebetween (e.g., 7 to 11 amino acids in length), not counting the histidines of the His-tag.

In various embodiments, the disclosure provides a vaccine composition comprising a pharmaceutical carrier and one or more liposomes comprising cobalt porphyrin-phospholipid conjugate, optionally phospholipids that are not conjugated to porphyrin, optionally sterols, and optionally polyethylene glycol (PEG), and having one or more MHC-I targeting peptides having a polyhistidine tag are incorporated into the bilayer such that a portion of the polyhistidine tag resides in the bilayer and at least a portion of the MHC-I targeting peptide is exposed to the exterior of the bilayer. In various embodiments, all or almost all of the MHC-I peptide is exposed to the exterior of the liposome and all or almost all of the polyhistidine tag resides in the bilayer. The vaccine composition may comprise a plurality of liposomes, each liposome comprising the same or different MHC-I peptide as another liposome in the composition. For example, a vaccine composition may comprise sets of liposomes, each liposome in a set comprising a specific MHC-I peptide, and each set comprising a different MHC-I peptide. In an embodiment, liposomes may comprise more than one MHC-I peptide and different liposomes in the composition may comprise different combinations of MHC-I peptides.

The present nanostructures can be used for generation of immune response or enhancement of immune response. The present cancer vaccine adjuvant can be used for generating anti-tumor responses using short, MHC-I restricted peptides. In an aspect, this disclosure provides methods for generating or enhancing an anti-tumor immune response. The method comprises administering to a subject in need of immunization, a composition comprising liposomes that comprises cobalt porphyrin-phospholipid conjugate, optionally phospholipids that are not conjugated to porphyrin, optionally sterols, and optionally polyethylene glycol (PEG), and having one or more MHC-I targeting peptides having a polyhistidine tag are incorporated into the bilayer such that a portion of the polyhistidine tag resides in the bilayer and at least a portion of the MHC-I targeting peptide is exposed to the exterior of the bilayer.

In some embodiments, the disclosure provides a method for increasing the immunogenicity of MHC-I peptides and/or eliciting neutralizing antibodies against MHC-I peptides by administering to a subject in need of treatment a composition comprising liposomes, wherein the liposomes comprise bilayers, which comprise cobalt porphyrin-phospholipid conjugate, optionally phospholipids that are not conjugated to porphyrin, optionally sterols, and optionally polyethylene glycol (PEG), and have one or more MHC-I peptides having a polyhistidine tag incorporated into the bilayer such that a portion of the polyhistidine tag resides in the bilayer and at least a portion of the MHC-I Peptide is exposed to the exterior of the bilayer. Optionally, one or more adjuvants may be incorporated into the nanostructures or administered separately.

In some embodiments, the present disclosure provides a method for in vivo cancer epitope screening and improvement using peptide microlibraries. The method comprises introducing one or more peptides into an animal model and subsequently assessing anti-cancer activity and/or CD8+ T cell activation generated by the one or more peptides. The peptides may be further improved by iteratively mutating residues and testing mutated peptides for improved activity. Combinations of peptides identified by the described screening may be used as multiplexed vaccines.

### BRIEF DESCRIPTION OF THE FIGURES

FIG. 1. CPQ liposomes rapidly and stably bind short MHC-I restricted peptides. A) Components of the CPQ adjuvant system used in this study with the model A5 peptide. The sequence of A5 is SEQ ID NO:1. B) Binding of liposomes to A5 peptide (with or without his-tag) following 1 hr incubation. C) Binding of A5 (with his-tag) to indicated liposomes following 1 hr incubation. D) and E) show hydrodynamic sizes of liposomes in (B) and (C), respectively. F) Cryo-electron micrographs of CPQ liposomes with or without A5 peptide bound. G) Binding kinetics of A5 to CPQ and 2HPQ liposomes. H) Refrigerated storage stability of CPQ and CPQ/A5 liposomes. I) Binding stability of A5 to CPQ and 2HPQ liposomes in the presence of 40% human serum with incubation at 37° C. Error bars show mean+/−std. dev. for n=3 independent experiments. * p<0.05, ** p<0.01, *** p<0.001, and **** p<0.0001, analyzed by two-way ANOVA (B, G, H, I), or one-way ANOVA (C) with Bonferroni multiple comparisons post-test.

FIG. 2. Sizes and polydispercities of liposomes after A5 binding. Peptide binding to liposomes with different mass ratio. A) Fluorescent A5 peptide was incubated with CPQ and 2HPQ liposome for 1 hr with a peptide to liposome mass ratio of 1:8, 1:4, 1:2 or 1:1. The fluorescence of A5 was quenched when bound with liposome. B) Binding of A5 peptide to liposomes by micro-centrifugation method. C) Sizes and D) polydispersity of liposomes after peptide binding. Error bar show mean+/−std. dev. for n=3 independent experiments.

FIG. 3. A5 admixed with CPQ liposome induces robust Ag-specific CD8⁺ T cell responses. BALB/c mice were immunized intramuscularly on day 0 and 7 with 500 ng A5 admixed with the indicated adjuvants. Ag-specific CD8⁺ T cells and effector-memory (Tem) phenotypes in the blood were then assessed by tetramer and surface marker staining. IFN-γ and TNF-α producing CD8⁺ T cells were assessed by intracellular staining of splenocytes. Flow cytometry gating (A) and percentage (B) of AH1 tetramer⁺ CD8⁺ T cells. T cell phenotype gating (C) and percentage (D) of Tem CD8⁺ T cells. IFN-γ producing CD8⁺ T cell gating (E) and percentage (F) in splenocytes after peptide re-stimulation. G) Percent of TNF-α producing CD8⁺ T cells in splenocytes after antigen re-stimulation. H) In vitro lysis of CT26 target cells (“T”) by T effector cells (“E”) from splenocytes of CPQ/A5-vaccinated mice or untreated mice at various E:T ratios. Error bars show mean+/−std. dev. for n=7 independent experiments for B, D, n=5 independent experiments for F, G and n=3 independent experiments for H. * p<0.05, ** p<0.01, *** p<0.001, and **** p<0.0001, analyzed by (B, D) one-way ANOVA, (H) two-way ANOVA with Bonferroni multiple comparisons post-test or (F, G) two-tailed unpaired Student's t test.

FIG. 4. CPQ/A5 vaccination elicits robust AH1 specific CD8⁺ T cells in the spleen. BALB/c mice were vaccinated intramuscularly with A5 admixed with indicated adjuvants on day 0&7. Each injection contained 0.5 pg antigen. On day 14, splenocytes were prepared from these mice and stained with antibodies. (A) Cells were first gated by SSC-FSC, then by CD8 positive but I-A/I-E, CD4, B220 negative. AH1-specific T cells were gated by AH1 tetramer positive cells. (B) Percent AH1 specific CD8⁺ T cells in the spleen of mice vaccinated with indicated vaccine. ****p<0.0001, analyzed by (B) one-way ANOVA.

FIG. 5. Durable and robust protection from diverse tumor model challenges with CPQ/A5 immunization. (A) Mice were immunized on day 0 and 7 with indicated vaccine dose and Ag-specific CD8⁺ T cells in blood were assessed on day 7 and 13 by tetramer staining. For each pair of bars, “Prime” is on the left and “Boost” is on the right. (B) Tumor-free days following CT26 challenge on day 14 following intramuscular immunization with CPQ/A5 on day 0 and day 7, at the indicated A5 dose. The study duration was 90 days. (C) Correlation of Ag-specific CD8⁺ T cells and tumor sizes 2 weeks after tumor inoculation. (D) AH1-specific CD8⁺ T cells in the blood on day 13 and spleen on day 13 (for each pair of bars, “Blood” is on the left and “Spleen” is on the right), and (E) tumor-free days after challenge with CT26 cells, for mice immunized with 500 ng A5 admixed with the indicated adjuvant. Percent of mice with tumor sizes smaller than 1 cm after immunization with 500 ng A5 admixed with CPQ or 2HPQ liposomes challenged subcutaneously with CT26 (F) or CMS4 (G) cells, or orthotopically with 4T07 (H) cells. (I) Kinetics of Ag-specific CD8⁺ T cell in the blood of mice vaccinated on days 0 and 7 with 500 ng A5 admixed with the indicated adjuvant. (J) Tumor growth of mice vaccinated with CPQ/A5 or 2HPQ/A5, 73 days after the final boost, when mice were challenged CT26 cells subcutaneously (indicated by arrow). Error bars show mean+/−std. dev. for n=5 mice per group. * p<0.05, ** p<0.01, *** p<0.001, and **** p<0.0001, analyzed by (B, E) one-way ANOVA, (A, D, I, J) two-way ANOVA with Bonferroni multiple comparisons post-test or long rank test (F, G, H).

FIG. 6. The effect of QS21 and antigen dosage to T cell production. CPQ liposome were prepared with mass ratio of [CoPoP:PHAD:QS21=1:1:1] or [CoPoP:PHAD:QS21=1:1:0.25], antigens were incubated with liposome with a mass ratio of 1:4. BALB/c mice were injected with CPQ/A5 vaccine on day 0&7, blood was collected for tetramer staining on day 14. Percent AH1 tetramer⁺ cells of CD8⁺ T cells in the blood of mice injected with indicated vaccine. Error bars show mean+/−std. dev. for n=3 independent experiments. * p<0.05, analyzed by one-way ANOVA with Bonferroni multiple comparisons post-test.

FIG. 7. Safety of CPQ/A5. CD-1 mice were untreated or vaccinated with CPQ/A5 on days 0 and 7 with 500 ng A5 admixed with CPQ liposomes containing 2 pg each of CoPoP, QS-21 and PHAD. Blood and organs were collected on day 14. (A) Body weight of CD-1 mice untreated or vaccinated with CPQ/A5. (B) Embedded hematoxylin and eosin stained slices of indicated organs. (C) Complete blood count parameters are as follows: WBC (white blood cells), NEU (neutrophils), LYM (lymphocytes), MONO (monocytes), EOS (eosinophils), BAS (basophils), RBC (red blood cell count), HGB (hemoglobin), HCT (hematocrit), MCV (mean cell volume), MCH (mean cell hemoglobin), MCHC (mean cell hemoglobin concentration), PLT (platelet), MPV (mean platelet volume), RDW (red cell distribution width) (“CPQ+A5” is the bar on the left and “Ctrl” is the bar on the right). (D) Serum markers with their general description are as follows: BUN (blood urea nitrogen), phosphorus, calcium, total protein, albumin, globulin, glucose, cholesterol, ALT (alanine aminotransferase), ALP (alkaline phosphatase) and total bilirubin. Values show mean+/−std. dev for n=5 mice per group. “ND”; no data provided for normal range. No statistically significant differences were observed in any groups (*P<0.05 based on One-way ANOVA) (“CPQ+A5” is the bar on the left and “Ctrl” is the bar on the right).

FIG. 8. Therapeutic efficacy of CPQ/A5 vaccination on early-stage CT26 cancers. BALB/c mice were inoculated with CT26 cells subcutaneously on day 0, then immunized with CPQ/A5 or 2HPQ/A5 (500 ng peptide) on days 5 and 12. Average tumor sizes (A) and tumor sizes of individual mice vaccinated with CPQ/A5 (B), 2HPQ/A5 (C) or untreated (D) and percent of mice with tumor sizes smaller than 1 cm following tumor inoculation (E), for n=5 mice per group. For the metastasis model, mice were injected intravenously with CT26 tumor cells, and then immunized 2 and 9 days later. Lungs were assessed for metastases on day 18. Metastases were present in the untreated and 2HPQ/A5 groups, but not in the CPQ/A5 group, as shown by a representative photograph (F), count of lung metastasis nodules (G) and the lung weights (H). Error bars show mean+/−std. dev. for n=5 mice per group. ** p<0.01, *** p<0.001, and **** p<0.0001, analyzed by (G, H) one-way ANOVA, (A) two-way ANOVA with Bonferroni multiple comparisons post-test or long rank test (E). Asterisks in panel A indicate statistically significant differences between CPQ/A5 and control group.

FIG. 9. Putative mechanism of CPQ/A5 immunization. (A) Schematic illustration of T cell activation following immunization with CPQ. (B) Immune cell populations in draining lymph nodes harvested two days after immunization with the indicated vaccination. For each series of bars, the left bar is “Untreated,” the middle bar is “CP/A5,” and the right part is “CPQ/A5.” (C) Uptake of A5 in murine macrophages or BMDCs following 1 hr incubation. For each series of bars, left is “BMDC” and right is “Macrophage.” (D) Uptake of A5 peptide in macrophages in the presence of the indicated phagocytosis and endocytosis inhibitors. (E) Confocal micrographs of murine BMDCs incubated with beads coated with CPQ or CPQ/A5-hilyte488 showing colocalization with H-2L^{d} (MHC-I) and LAMP-1. Scale bar, 10 μm. (F) Binding of A5 to liposomes containing CoPoP or CoNTA, followed by the addition of serum and lysosome extract as indicated. Error bars show mean+/−std. dev. for n=5 for lymph node studies and n=3 independent experiments for macrophage and BMDC uptake studies and A5 binding studies. ** p<0.01, ***p<0.001, and **** p<0.0001, analyzed by (D) one-way ANOVA, (B, C) two-way ANOVA with Bonferroni multiple comparisons post-test.

FIG. 10. Liposomes drain to lymph nodes rapidly. BALB/c mice were intramuscularly injected with 50 μL 320 pg/mL 2HP on the left leg, and 0, 1, 2 and 4 hr after vaccination, lymph nodes were collected from mice for fluorescent imaging. (A) Indication of lymph nodes in the mice. (B) Fluorescence of lymph nodes. (C) Number of fluorescent lymph nodes at indicated time point. Error bars show mean+/−std. dev. for n=3 mice. * p<0.05, analyzed by one-way ANOVA with Bonferroni multiple comparisons post-test.

FIG. 11. Macrophage uptake of CPQ/A5-Hilyte488 liposome. RAW264.7 macrophages were incubated with CPQ/A5, 2HPQ/A5 or A5 alone (1 μg/mL antigen concentration) for 1 hour, then washed by PBS and lysed by 0.1% triton. (A) Percent macrophage uptake of A5-Hilyte488 were measured at indicated time point. (B) Fluorescent images of macrophages, CPQ/A5, 2HPQ/A5 and A5 alone were incubated with macrophages for one hour. (C) Percent release of peptide in the microphage. Error bars show mean+/−std. dev. for n=3 independent experiments. ** p<0.01, *** p<0.001, analyzed by two-way ANOVA with Bonferroni multiple comparisons post-test. Asterisks in panel A indicate statistically significant differences between CPQ/A5 and A5 group.

FIG. 12. PoP lipids coated on silica beads. Silica beads were uncoated or coated with 2HPQ, then subject to fluorescence microscopy.

FIG. 13. Integrity of fluorescent A5 peptide uptaken by macrophages. Macrophages were incubated with CPQ/A5 for an hour or 2 hours then lysed by lysis buffer and subject to HPLC.

FIG. 14. Impact of ERR-tag and his-tag length of A5 on H-2L^{d} binding and inducing CD8⁺ T cells. (A, B) in vitro H-2L^{d} binding competing between A5 (no his-tag and no ERR-tag) and indicated peptides with different concentration. The sequences in FIG. 14 (A) with 6 Histidines are SEQ ID NO:2. The sequences in FIG. 14 (B) with 6 Histidines are SEQ ID NO:2, and 5 Histidines are SEQ ID NO:3. (C) Left panel; Ec50 of indicated peptides competing with A5 in terms of in vitro H-2L^{d} binding. BALB/c mice were vaccinated with CPQ and indicated peptides on day 0&7, then blood was collected for AH1 tetramer staining on day 14. Right panel; percent of AH1 tetramer⁺ cells of CD8⁺ T cells in the blood of mice vaccinated with CPQ and indicated peptides. Error bars show mean+/−std. dev. for n=3 independent experiments. The sequence in FIG. 14 (C) with 6 Histidines is SEQ ID NO:2, and 5 Histidines is SEQ ID NO:3.

FIG. 15. Impact of length of his-tag on A5 peptide binding and immunogenicity. BALB/c mice were injected with A5 peptide with indicated length of his-tag and CPQ or CQ on days 0 & 7, then blood was collected for AH1 tetramer staining on day 14. (A) Binding of peptide to CPQ and 2HPQ liposomes. (B) Sizes of CPQ liposomes after binding. (C) Percent AH1 tetramer⁺ cells of CD8⁺ T cells in blood of mice injected with indicated peptide combined with indicated adjuvant. Error bars show mean+/−std. dev. for n=3 independent experiments. * p<0.05, ** p<0.01, and **** p<0.0001, analyzed by two-way ANOVA with Bonferroni multiple comparisons post-test.

FIG. 16. Short peptide micro-library screening with CPQ reveals the RragcL385P 9mer peptide as a functional vaccine epitope to inhibit CT26 and 4T1 lung metastasis. (A) Approach used for in vivo screening of a 100 peptide micro-library. Mice were immunized with pooled micro-library peptides (5 peptides at a time, along with A5 serving as an internal control). Collected splenocytes were then re-stimulated with individual short peptides and IFN-γ was measured relative to A5 re-stimulation to indicate Ag-specific T cell presence. The sequences in FIG. 16 (A) with 6 Histidines are SEQ ID NO:2. (B) Identification of immunogenic peptides. Error bars show data range of triplicate wells from n=2 mice per group, expressed relative to the IFN-γ produced by A5 in the same immunization group. C) Mice were intravenously challenged with CT26 cells on day 0, and then immunized with CPQ with RragcL385P, Eml5G44R, Tmem5S71N or the combination 1 and 8 days later (1000 ng total peptide). Lung nodules (C) and weight (D) were assessed on day 18. The challenge was repeated but immunization was with 500 ng RragL385P with the indicated adjuvants, and lung metastases were assessed following challenge with CT26 (F) or 4T1 (H) cells. Images of lungs from different groups were taken (E). Lung weights also were assessed for CT26 (G) and 4T1 (I) tumor-bearing mice. Lines show mean for n=5 mice per group. * p<0.05, ** p<0.01, *** p<0.001, and **** p<0.0001, analyzed by one-way ANOVA with Bonferroni multiple comparisons post-test.

FIG. 17. Cytotoxicity of splenocytes from CPQ/A5 and CPQ/RragcL385P vaccinated mice. Effector splenocytes (E) were incubated with 5000 target CT26 cells (T) for 5 hours. Then specific lysis was analyzed by the nonradioactive LDH release assay by following the manufacturer's instructions. Error bars show mean+/−std. dev. for n=4 independent experiments.

FIG. 18. Identification of RENCA neo-antigens. (A) Approach used for in vivo screening of RENCA neo-antigen. (B) Flowcharts for the number of genomic variations or variant peptides identified at each stage of analysis, and finally the number of peptides validated as immunogenic.

FIG. 19. 20 RENCA neo-antigen candidates admix with CPQ liposome as a prophylactic vaccine induced CD8⁺ T cell response and inhibited tumor growth. BALB/c mice were immunized with CPQ/peptides vaccine on day 0 & 7, then inoculated with RENCA cells subcutaneously on day 14. 21 days after tumor inoculation, spleens were collected and splenocytes were prepared and stimulated with injected peptide individually for IFN-γ, TNF-α and TEM cell staining. (A) Binding percentage of 20 predicted peptides to CPQ and 2HPQ liposomes. Sizes (B) and polydispersity (C) of liposomes with or without peptides binding. For each series of bars, “CPQ” is on the left and “2HPQ” is on the right. (D) Tumor growth of CPQ/peptides and 2HPQ/peptides vaccinated mice. (E) Tumor growth of individual mice with CPQ/peptides vaccination (E) or 2HPQ/peptides vaccination (F). G) Percent TEM cells in CD8⁺ T cells in spleen. H) Percent IFN-γ producing CD8⁺ T cells in the spleen. Error bars show mean+/−std. dev. for n=5 independent experiments.

FIG. 20. CPQ/RENCA peptide 2 vaccine inhibited tumor growth in mice. BALB/c mice were immunized with CPQ/peptide vaccine on day 0 & 7, then inoculated with RENCA cells subcutaneously on day 14. (A) Tumor sizes of mice vaccinated with CPQ admixed with indicated peptide 18 days post tumor inoculation. Splenocytes were prepared 18 days post tumor inoculation and cultured in vitro for 5 days then stimulated with antigens and served as Effector cells (E), tumor cells were target cells (T). Effector cells were incubated with target cells for 5 hours, then specific lysis was analyzed by the nonradioactive LDH release assay by following the manufacturer's instructions. Percent cell lysis of RENCA cells (B) and percent cell lysis of irrelevant TC-1 cells (C). Error bars show mean+/−std. dev. for n=5 independent experiments.

FIG. 21. E6/E7 MHC-I epitope candidates form immunogenic particles that inhibit TC-1 tumor growth. (A) Approach for screening for functional MHC-I restricted epitopes by DNA sequencing the E6/E7 oncogenes; predicting MHC-I epitopes; using CPQ to form a multivalent vaccine; and immunizing mice to assess functional immunogenicity. (B) Binding of the 6 synthetized peptides identified to CPQ or 2HPQ liposomes. C57BL/6 mice were vaccinated with the multivalent vaccine on days 0 and 7; then on day 14, blood was collected and central memory (C) and effector memory phenotypes (D) within the gated CD8⁺ T cell population was assessed. (E) Epitope-specific, IFN-γ producing CD8⁺ T cells from PBMCs of mice vaccinated with the multiplexed vaccine. (F) Tumor growth in mice challenged with TC-1 cells following immunization with the multiplexed vaccine. Error bars show mean+/−std. dev. for n=3 per group for binding and size studies and n=5 per group for mice studies. * p<0.05, *** p<0.001 and **** p<0.0001, analyzed by (E, F) two-way ANOVA with Bonferroni multiple comparisons post-test or (B, C, D) two-tailed unpaired Student's t test.

FIG. 22. All E6/E7 synthetic epitopes formed particles, but only E7₄₉₋₅₇ inhibited TC-1 tumor growth. (A) Schematic of single peptide vaccines assessed to identify functional eptitopes. The sequences in FIG. 22 (A) for single peptide vaccine epitopes are, from left to right, SEQ ID NOs:4-9. Binding (B) and size (C) of predicted HPV-16 E6/E7 epitopes to CPQ and 2HPQ liposomes. Peptides were incubated for 1 hr at room temperatures with liposomes prior to measurement. Error bars show mean+/−std. dev. for n=3. Mice were immunized with 500 ng of each peptide mixed with CPQ on days 0 and 7 and subjected to TC-1 tumor challenge on day 14. Mean tumor volume (D) and percentage of mice with tumors size less than 1 cm (E) for n=5 mice per group. All mice in the CPQ/E7₄₉₋₅₇ group were tumor free at the end of the study.

FIG. 23. Immunization with E7_{HHH49-57}-admixed with CPQ inhibits tumor growth in a therapeutic setting more potently than poly(I:C). C57BL/6 mice were inoculated with TC-1 cells subcutaneously on day 0; then different vaccines were given 2 days post-tumor inoculation. Blood was collected 18 days post-tumor inoculation for CD8⁺ T cell analyses. (A) Tumor growth of mice that vaccinated with indicated vaccine. (B) Tumor sizes of mice on day 19. (C) Survival of mice. (D) Percentage of CD8⁺E7₄₉₋₅₇ tet⁺ cells in the blood. Error bars show mean+/−std. dev. for n=5 per group. * p<0.05, ** p<0.01 and **** p<0.0001, analyzed by (C) long rank test or (D) one-way ANOVA with Bonferroni multiple comparisons post-test.

FIG. 24. Developing a Trp2 e-mimotope with CPQ with improved function compared to the native epitope. (A) A two-step approach for developing of mimotopes using positional micro-libraries with in vivo screening and tumor challenge guiding the sequence selection. (B) C57BL/6 mice were immunized with peptide libraries with random amino acids at the indicated residue on day 0 and 7, then were challenged with B16-F10 cells on day 14. (C) Data show the day 21 tumor volume. Error bars show mean+/−std. dev. for n=3 independent experiments.

FIG. 25. Trp2_8C and Trp2_8Y have better anti-tumor efficacy compares to native Trp2_8W peptide when admixed with CPQ as a prophylactic vaccine. Mice were immunized with Trp2 peptides with indicated amino acid mutation at position 8. Data show tumor volume on day 21 following B16-F10 challenge. Tumor growth (A), survival (B) and day 23 tumor volume (C) of untreated mice or mice vaccinated with mimotopes (Trp2-8C and Trp2-8Y) or the native sequence (Trp2-8W). Error bars show mean+/−std. dev. for n=5 independent experiments. * p<0.05, ** p<0.01, *** p<0.001, and **** p<0.0001, analyzed by long rank test (B) or one-way ANOVA (C) with Bonferroni multiple comparisons post-test.

FIG. 26. Env₃₇₋₄₄ position-scanning peptide libraries as particle immunogens. Scheme of experimental design of demonstrating positional library as efficient immunogens. The sequences in the far left schematic are, from top to bottom, SEQ ID NOs:10-18.

FIG. 27. Env₃₇₋₄₄-Pos5 positional peptide vaccine induced T cells that bind with Env₃₇₋₄₄ tetramer and has high affinity to Env₃₇₋₄₄-Pos5 positional peptides and enhanced affinity to Env₃₇₋₄₄ peptide. BALB/c mice were untreated or vaccinated with CPQ and indicated peptide libraries on days 0 & 7, then blood was collected on day 14 for tetramer staining and analysis; splenocytes were prepared on day 21 for analysis. (A) NetMHC binding percentile of Env₃₇₋₄₄ positional libraries, dash line indicates the binding percentile of Env₃₇₋₄₄ peptide to H-2L^{d} (B) Percentage of Env₃₇₋₄₄ tet⁺ cells in the CD8⁺ T cell population in blood. (C) Percentage of effector-memory T cell phenotype in CD8⁺ T cell population. Splenocytes were stimulated with 10 μg mL⁻¹ Env₃₇₋₄₄ or Env₃₇₋₄₄-Pos5, then intracellular IFN-γ were stained and analyzed by flow cytometry. Flow cytometry gating (D) and percentage (E) of IFN-γ producing cells in the CD8⁺ T cell population after Ag stimulation. * p<0.05, and **** p<0.0001, analyzed by two-way ANOVA (E) with Bonferroni multiple comparisons post-test. Error bars show mean+/−std. dev. for n=5 per group. For each series of bars in (E), “Env₍₃₇₋₄₄₎” is on the right and “Pos5” is on the left.

FIG. 28. AH1 positional peptide libraries and library-mixtures as particle immunogens. Scheme of CPQ vaccines that made of one AH1 positional library or 4 AH1 positional libraries. The sequences in the far left schematic are, from top to bottom, SEQ ID NOs:19-28.

FIG. 29. AH1-Pos1, Pos3, Pos5 and Pos8 library vaccine immunogens induced AH1-specific T cells better than the wild-type epitope and protected mice from tumor challenging. BALB/c mice were untreated or vaccinated with CPQ and indicated peptide or peptide libraries on days 0 & 7, then blood was collected for analysis and CT26 tumor cells were inoculated subcutaneously on day 14. (A) AH1 positional library MHC-I binding percentile, dash line indicates the binding percentile of AH1 peptide to H-2L^{d}. (B) Percentage of AH1 tet⁺ cells and TEM cells (C) in the CD8⁺ T cell population. (D) Tumor free days of mice vaccinated with CPQ and indicated positional libraries. Error bars show mean+/−std. dev. for n=3 per group.

FIG. 30. Identification of AH1 peptides that has anti-tumor efficacy. Mice were immunized with AH1 peptides with indicated amino acid replacement on position 1 or 3 or 5 or 8 on day 0 & 7, then blood was collected for AH1 tetramer analysis and mice were challenged with CT26 cells subcutaneously on day 14. Tumor sizes of mice vaccinated with AH1 peptide with amino acid replacement at position 1 (SEQ ID NO:20) (A), position 3 (SEQ ID NO:22) (B), position 5 (SEQ ID NO:24) (C) or position 8 (SEQ ID NO:27) (D). Data show tumor volumes on day 28 following CT26 challenge.

### DESCRIPTION OF THE DISCLOSURE

Throughout this application, the use of the singular form encompasses the plural form and vice versa. For example, “a”, or “an” also includes a plurality of the referenced items, unless otherwise indicated.

Where a range of values is provided in this disclosure, it should be understood that each intervening value, and all intervening ranges, between the upper and lower limit of that range is/are also included, unless clearly indicated otherwise. The upper and lower limits from within the broad range may independently be included in the smaller ranges encompassed within the disclosure.

The term “therapeutically effective amount” as used herein refers to an amount of an agent or composition sufficient to achieve, in single or multiple doses, the intended purpose of treatment. Treatment does not have to lead to complete cure, although it may. Treatment can mean alleviation of one or more of the symptoms or markers of the indication. The exact amount desired or required will vary depending on the particular compound or composition used, its mode of administration, patient specifics and the like. Appropriate effective amount can be determined by one of ordinary skill in the art informed by the instant disclosure using only routine experimentation. Within the meaning of the disclosure, “treatment” also includes prophylaxis and treatment of relapse, as well as the alleviation of acute or chronic signs, symptoms and/or malfunctions associated with the indication. Treatment can be orientated symptomatically, for example, to suppress symptoms. It can be effected over a short period, over a medium term, or can be a long-term treatment, such as, for example within the context of a maintenance therapy. Administrations may be intermittent, periodic, or continuous.

The term “MHC-I restricted peptide” or “MHC-I targeting peptide” or “MHC-I binding peptide” are used interchangeably and refer to 4-11 mer peptides that are able to present on the MHC-I molecule and induce CD8⁺ T cell response.

Short MHC class-I restricted (MHC-I) peptides contain the minimal biochemical information to induce antigen (Ag)-specific CD8⁺ cytotoxic T lymphocytes (CTLs), but are generally ineffective in doing so without an adjuvant platform. In this disclosure, we describe a novel cobalt-porphyrin liposome vaccine adjuvant that induces rapid particleization of conventional, short MHC-I epitopes, leading to highly functional cellular responses with nanogram dosing. To demonstrate the effectiveness of the present liposomes, as further described in the examples, we carried out studies in mice, where immunization with a short MHC-I peptide derived from the gp70 oncogene as a model system generated functional, Ag-specific CD8⁺ T cells, resulting in rejection of multiple tumor cell lines, durable immunity, and control of local and metastatic disease. In vivo screening of peptide micro-libraries comprising a hundred putative MHC-I binding cancer epitopes revealed that a handful were immunogenic. To identify relevant human peptides, one or more neo-epitoes predicted to be MHC-I binders (using tumor cells or cell lines or by tumor-specific gene sequencing) may be incorporated into the present liposomes and their ability for generating or enhancing a functional immune response (e.g., inhibiting tumor growth) may be evaluated. A similar method may be used to identify relevant human peptides by using transgenic mice expressing HLA MHC molecules.

To address the challenge of poorly immunogenic target epitopes, positional micro-libraries can be developed to identify novel, enhanced peptide “e-peptide” with enhanced function compared to the native epitope. Further description is provided in the examples.

The present disclosure provides liposomes comprising MHC-I binding peptides, such as human MHC-I restricted peptides. The peptides may be native peptides or may be analogs thereof. The analogs may comprise modifications of the native peptides such that one or more functionalities of the peptides are altered. For example, the native peptides may be modified to improve binding to MHC-I; or stabilize the CD8⁺ TCR-peptide MHC-I complex or both. These modified constructs, which are altered peptide ligands, and termed herein as “e-mimotope”, can induce improved functional responses compared to the native peptide. There are several examples in the literature of “e-mimotope”, such as the A5 peptide in the case of mouse, which is derived from the AH1 epitope found in the gp70 glycoprotein (amino acid 423-431), a tumor-associated Ag expressed in various murine cancer cell lines. However, synthetic short A5 peptides conjugated to lipids and proteins display limited efficacy (Goodwin et al., Vaccine 35, 2550-2557, 2017; Zhang et al., Nature Communications 11, 1187, 2020). E-mimotope have been translated to clinical trials for cancer vaccines, such as the melanoma antigen Melan-A/MART-1₂₆₋₃₅ A27L, gp100 2M, NY-ESO-1 C165V, and Survivin T97M (ELMLGEFLKL (SEQ ID NO:29)).

In the present disclosure, using a next-generation vaccine adjuvant, we demonstrate that short, conventional MHC-I restricted synthetic peptides (without further covalent conjugation) can also address at least three challenges of peptide cancer vaccine development: (1) potently induce functional CD8⁺ T cells using simple short peptides; (2) enable novel functional epitope discovery via peptide micro-library screening; and (3) improve responses to established epitopes via bettertope evolution using mutated peptide libraries.

In an aspect, the present disclosure provides a composition comprising a pharmaceutical carrier and one or more liposomes comprising cobalt porphyrin-phospholipid conjugate, optionally phospholipids that are not conjugated to porphyrin, optionally sterols, and optionally polyethylene glycol (PEG), and having one or more MHC-I targeting peptides having a polyhistidine tag incorporated into the bilayer such that a portion of the polyhistidine tag resides in the bilayer and at least a portion of the MHC-I targeting peptide is exposed to the exterior of the bilayer. In various embodiments, all or almost all of the MHC-I peptide is exposed to the exterior of the liposome and all or almost all of the polyhistidine tag resides in the bilayer. The vaccine composition may comprise a plurality of liposomes, each liposome comprising the same or different MHC-I peptide as another liposome in the composition. For example, a vaccine composition may comprise a plurality of liposomes, each comprising the same one or more MHC-I binding peptide(s), or the vaccine composition may comprise a plurality of sets of liposomes, each liposome in a set comprising a specific one or more MHC-I peptide(s), and each set comprising a different one or more MHC-I peptide(s). In various embodiments, liposomes may comprise peptides with the same sequence or different sequences, and different liposomes in the composition may comprise different combinations of MHC-I peptides. The compositions may further comprise adjuvants, which may be incorporated into the liposomes, or may be present in the composition, but not incorporated into the liposomes.

In an aspect, this disclosure provides methods for generating or enhancing an anti-tumor immune response. The method comprises administering to a subject in need of immunization, a composition comprising liposomes that comprises cobalt porphyrin-phospholipid conjugate, optionally phospholipids that are not conjugated to porphyrin, optionally sterols, and optionally polyethylene glycol (PEG), and having one or more MHC-I targeting peptides having a polyhistidine tag are incorporated into the bilayer such that a portion of the polyhistidine tag resides in the bilayer and at least a portion of the MHC-I targeting peptide is exposed to the exterior of the bilayer.

In various embodiments, the disclosure provides a method for increasing the immunogenicity of MHC-I peptides and/or eliciting neutralizing antibodies against MHC-I peptides by administering to a subject in need of treatment a composition comprising liposomes, wherein the polyhistine-tagged MHC-I peptides are incorporated into the liposomes, wherein the liposomes comprise bilayers, which comprise cobalt porphyrin-phospholipid conjugate, optionally phospholipids that are not conjugated to porphyrin, optionally sterols, and optionally polyethylene glycol (PEG), such that a portion of the polyhistidine tag resides in the bilayer and at least a portion of the MHC-I Peptide is exposed to the exterior of the bilayer. Optionally, one or more adjuvants may be incorporated into the nanostructures or administered separately.

In an embodiment, this disclosure provides a method of inducing tumor antigen-specific CD8⁺ T cell responses capable of recognizing the antigen on cancer cells comprising administering to an individual in need of treatment a composition comprising liposomes, wherein polyhistidine-tagged MHC-I binding peptides from the tumor antigen are incorporated into the liposomes, wherein the liposomes comprise bilayers, which comprise cobalt porphyrin-phospholipid conjugate, optionally phospholipids that are not conjugated to porphyrin, optionally sterols, and optionally polyethylene glycol (PEG), such that a portion of the polyhistidine tag resides in the bilayer and at least a portion of the MHC-I binding peptide is exposed to the exterior of the bilayer.

In an embodiment, the disclosure provides a method of treating an individual who is afflicted with a tumor comprising administering to the individual a composition comprising liposomes that comprises cobalt porphyrin-phospholipid conjugate, optionally phospholipids that are not conjugated to porphyrin, optionally sterols, and optionally polyethylene glycol (PEG), and having one or more MHC-I targeting peptides having a polyhistidine tag are incorporated into the bilayer such that a portion of the polyhistidine tag resides in the bilayer and at least a portion of the MHC-I targeting peptide is exposed to the exterior of the bilayer. The composition may be administered one time or multiple times. For example, the treating clinician may follow the growth of the tumor and may adjust the dose and the frequency of administration of the composition.

In some embodiments, the present disclosure provides a method for in vivo cancer epitope screening and improvement using peptide microlibraries.

In embodiments, the disclosure provides for methods for screening a plurality of peptides to determine MHIC-1 binding, CD8+ T cell activation, anti-cancer activity or a combination thereof. The plurality of peptides may comprise a peptide library. In embodiments, the library comprises 2 or more peptides. In embodiments, the library comprises 2-1,000 peptides, inclusive, and including all ranges of numbers there between. In embodiments, the library comprises 2-100 peptides. Pluralities of peptides that comprise 2-100 peptides are referred to from time to time in this disclosure as microlibraries.

The peptides screened in the library can be from any source, or can be designer peptides. In certain embodiments, the peptides comprise or consist of amino acid sequences that are obtained or derived from neoantigens. In certain embodiments, the peptides are segments of neoantigens. In certain embodiments, the peptides are derivatives of segments of neoantigens. In certain embodiments, the peptides are designed based at least in part on predictions made using a computer implemented analysis, non-limiting examples of which are described herein.

In embodiments, peptides in the library used in a screen may be generated using a personalized medicine approach. This comprises determining polynucleotide sequences from cancer cells of an individual to identify encoded neoantigens, producing peptides based on identified neoantigens, and testing the peptides to determine one or more anti-cancer related characteristics of the tested peptides. Candidate peptides may be further characterized as described below. One or more peptides with demonstrated anti-cancer activity are used as prophylactic or therapeutic anti-cancer agents in the individual from whom the sample was obtained.

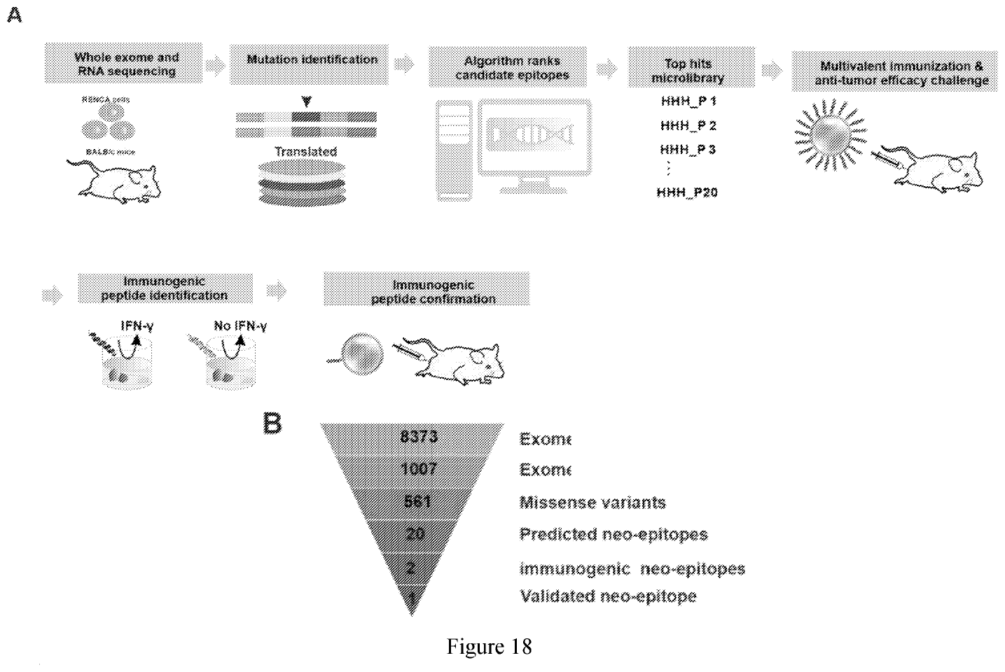

In embodiments, screening peptides is performed as follows. One or more peptides are introduced into an animal to assess the capability of the one or more peptides to stimulate a CD8+ T cell response, which indicates the one or more peptides bind to MIC-I and are candidates for use as anti-cancer agents. Various methods for determining whether or not any particular peptide can stimulate a CD8+ T cell response are known in the art and can be adapted for use in the described methods when given the benefit of the present specification. In one aspect, one or more peptides are introduced into an animal, such as a mouse model, after which cells from the animal tested in the presence of the one or more peptides that were introduced into the mouse. In certain approaches a mouse model comprising a humanized immune system can be used. Mouse models comprising humanized immune systems are commercially available, such as from THE JACKSON LABORATORY. In embodiments, the mouse model produces immune cells that express HLA-1 instead of MHC-I. In one embodiments, peptides introduced into the animal model stimulate IFN-γ production by CD8⁺ T cells within, or isolated from, the spleen of the animal, which indicates the peptides bind to MHC-1 and are candidates for use as anti-cancer vaccines. A representative overview of this process is provided in FIG. 18. FIG. 18 demonstrates whole exome and RNA sequencing. As will be recognized by those skilled in the art, exome sequencing comprises sequencing the portion of the genome that comprises exons. By using this approach and comparing the obtained exome sequences to a reference sequence, such as a non-cancer gene(s) control sequence, mutations in exons that are associated with cancer can be identified. The mutations can be evaluated using, for example, a computer implemented algorithm, including but not necessarily limited to a neural network simulation algorithm. The mutations may be any mutations that alter the protein coding sequence, including but not limited to mutations that cause alternatively or improperly spliced exons, missense mutations, nonsense mutations, frameshift mutations, insertions, and deletions, e.g., indels. The peptide evaluation can include a ranking of peptides based on a number of criteria, including but not necessarily limited to the predicted capability of the peptides to stimulate CD8⁺ T cell-mediated tumor cell lysis of a cancer cells that produce a protein that comprises an epitope that is comprised by the peptide. Further characterization of this and/or other capabilities of the peptides can be performed by immunization of an animal with one or more of the identified peptides to determine if any of the peptides can stimulate an anti-cancer response in an animal that is challenged with a cancer that produces a protein that includes an epitope comprised by the immunizing peptide. As illustrated in FIG. 18, the disclosure includes analysis of a plurality of distinct peptides to identify peptides that may be combined to produce, for example, a multivalent vaccine that comprises a plurality of identified peptides.

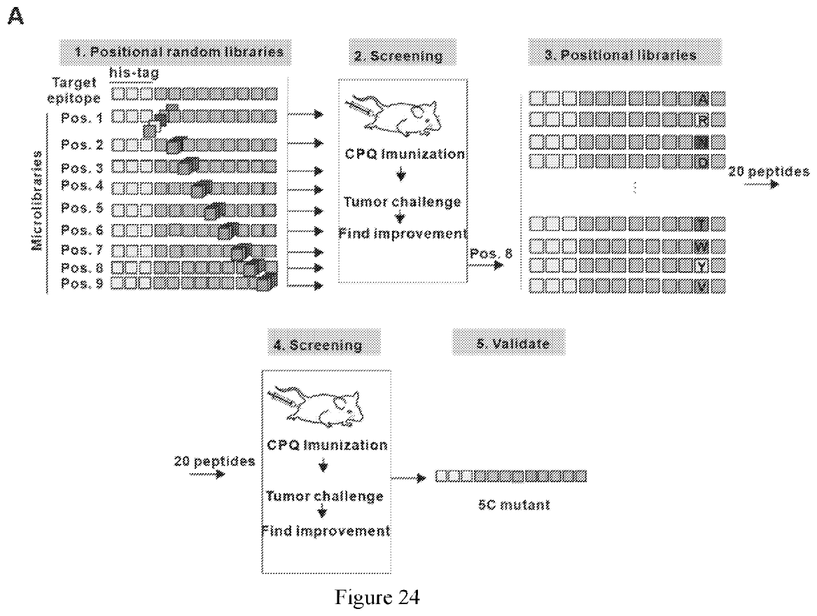

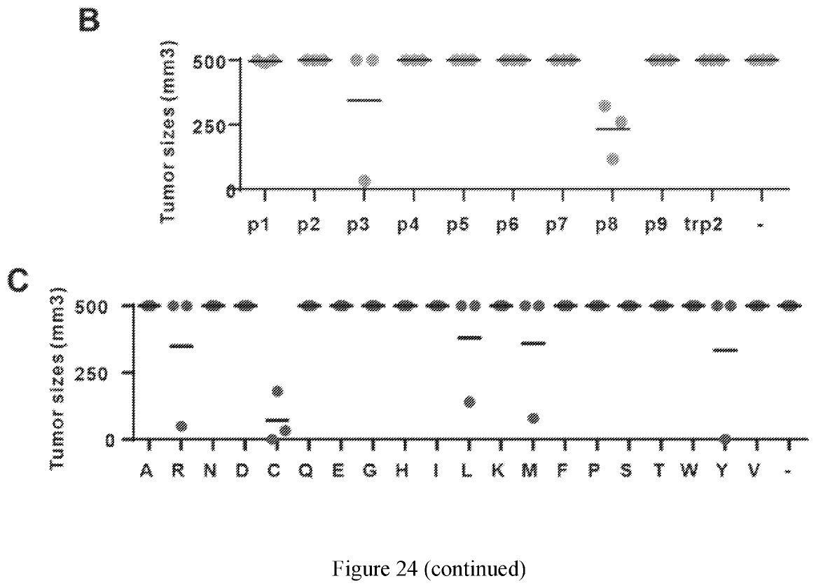

The disclosure further comprises improving the identified peptides by altering one or more amino acids in the identified peptides. A representative embodiment of this approach is shown in FIG. 24. As shown in FIG. 24, a peptide can be randomized at certain positions to produce a plurality of mutated peptides which are tested for changes in anti-cancer activity. For example, the disclosure provides for generating positional random libraries, screening positionally randomized peptides to select a first set of peptides, and if desired, making additional changes to the selected first set of peptides to provide a second set of peptides. The second set of peptides can be tested to identify one or more peptides that are further improved in one or more anti-cancer properties, relative to peptides in the first set.

An anti-cancer improvement in one or more peptides can be compared to any suitable reference value. In embodiments, the reference value is an anti-cancer effect elicited using a control peptide. The control peptide can be a native peptide, e.g., a wild type amino acid sequence, or a peptide that has a known anti-cancer effect that is compared to a peptide described herein, or a peptide identified by a method of the disclosure. The improved anti-cancer effect can be any anti-cancer effect. In embodiments, the anti-cancer effect is at least one of: an inhibition of growth of cancer cells, a reduction in tumor volume, an inhibition of metastasis, an inhibition of cancer relapse, a prolongation of survival, an improved response to a companion therapy used with the peptides, such as an adoptive immunotherapy, a chemotherapy, an antibody-based therapy, a CAR T therapy, or a checkpoint inhibitor therapy. An anti-cancer improvement in one or more peptides can also include improved activation of CD8+ T cells and/or improved affinity for MHC-I.

As discussed above, while this disclosure refers mostly to use of the described peptides with liposomes having bilayers or monolayers, the disclosure and various embodiments are also applicable to monolayers. The bilayers or monolayers are sometimes referred to herein as “membranes”.

Some or all of the cobalt porphyrins in the monolayer or bilayer of the liposome can non-covalently bind polyhistidine-tagged molecules, such that at least part of the polyhistidine tag of the tagged molecule resides within the bilayer and the tagged molecule is presented on the surface of the bilayer or otherwise exposed to the exterior of the liposome. It is considered that one or more histidine residues in the polyhistidine tag are coordinated to the cobalt metal within the bilayer. The imidazole groups of histidine residues of a polyhistidine tag may be coordinated to the cobalt metal bound to the porphyrin in the membrane. The entire histidine tag may reside within the bilayer. A porphyrin phospholipid conjugate that has cobalt metal conjugated thereto is referred to herein as CoPoP. Liposomes wherein the bilayer comprises CoPoP are referred to herein as CoPoP liposomes. The CoPoP liposomes can be functionalized with histidine tagged molecules. The term “His-tagged molecules” as used herein means molecules-such as, for example, MHC-I restricted peptides-which have a histidine tail. For example, a peptide with a histidine tail is a his-tagged molecule. Such his-tag containing CoPoP liposomes are referred to herein as His-tagged CoPoP liposomes or His-tagged CoPoP.

As used herein, “phospholipid” is a lipid having a hydrophilic head group having a phosphate group connected via a glycerol backbone to a hydrophobic lipid tail. The phospholipid comprises an acyl side chain of 6 to 22 carbons, including all integer number of carbons and ranges therebetween. In certain embodiments, the phospholipid of the porphyrin conjugate is 1-palmitoyl-2-hydroxy-sn-glycero-3-phosphocholine. The phospholipid of the porphyrin conjugate may comprise, or consist essentially of phosphatidylcholine (PC), phosphatidylethanoloamine (PE), phosphatidylserine (PS) and/or phosphatidylinositol (PI). Examples of phospholipids include, but are not limited to, Dipalmitoylphosphatidylcholine (DPPC), Dioleoyl phosphatidylcholine (DOPC), Dimyristoylphosphatidylcholine (DMPC), Distearoylphosphatidylcholine (DSPC), Distearoyl phosphatidylethanolamine (DSPE) and the like.

In certain embodiments, the porphyrin is conjugated to the glycerol group on the phospholipid by a carbon chain linker of 1 to 20 carbons, including all integer number of carbons therebetween.

The CoPoP bilayers functionalized with His-tagged MHC-I restricted peptides provide a platform for generation of specific immune responses to those His-tagged peptides. The His-tagged peptides are non-covalently attached to (coordinated to) the CoPoP and can be prepared by an incubation process. The process of preparation of the CoPoP does not require removal of reactive moieties—such as maleimide and the like—or exogenous catalysts or non-canonical amino acids that are used in other types of conjugation chemistries.

The cobalt-porphyrin may be in a bilayer or monolayer of self-assembling liposomes enclosing there within an aqueous compartment. Cobalt-porphyrin phospholipid (CoPoP) behaves like a conventional lipid with respect to its amphipathic nature. Therefore, monolayers or bilayers comprising CoPoP can be used for coating of nanoparticles by methods that are known to those skilled in the art. In one embodiment, the bilayer or monolayer of the present disclosure may be present on other nanoparticles, such as, for example, in the form of a coating. In one embodiment, the bilayer or monolayer containing cobalt-porphyrin (e.g., cobalt porphyrin-phospholipid) is present as a coating on gold or silica nanoparticles, or other nanoparticles with a hydrophilic surface. In one embodiment, the coating may be in the form of monolayers. In one embodiment the monolayer or bilayer containing cobalt-porphyrin (e.g., cobalt porphyrin-phospholipid) is present as a coating on hydrophobic surfaces such as carbon nanotubes. In one embodiment, the monolayers may form micelles surrounding one or more hydrophobic molecules.

The liposomes of the present disclosure comprise: i) porphyrin that has cobalt coordinated thereto forming cobalt-porphyrin, and wherein some or all of the cobalt porphyrin may be conjugated to a phospholipid to form a cobalt porphyrin-phospholipid conjugate, and ii) optionally, phospholipids that are not conjugated to the cobalt-porphyrin. Optionally, the liposomes may further comprise one or more of the following: sterols (e.g., cholesterol (abbreviated throughout as Chol), adjuvants (e.g., PHAD, QS-21, and the like, and combinations thereof), or oligoethers (e.g., PEG). For example, for a liposome comprising CoPoP/PoP, DOPC, Chol, PHAD, and QS-21, a typical mass ratio of [DOPC:Chol:CoPoP/PoP:PHAD:QS-21] is either [20:5:1:1:1] or [20:5:1:0.25:0.25].

The monolayer or the bilayer need not contain any phospholipids that are not conjugated to cobalt porphyrin and in this case only has cobalt porphyrin phospholipid conjugates. The cobalt porphyrin phospholipid can make up from 1 to 100 mol % of the monolayer or the bilayer, including 0.1 mol % values and ranges therebetween. For example, the cobalt porphyrin can make up from 1 to 20 mole %, or from 5 to 10 mol % of the monolayer or the bilayer. If the cobalt porphyrin makes up 100% of the monolayer or the bilayer, then there are no phospholipids present that are not conjugated to cobalt porphyrin. The bilayer or the monolayer can also comprise sterols and/or polyethylene glycol. The sterols can be cholesterol.

The histidine tag (His-tag) may carry a variety of MHC-I restricted peptides of interest for various applications. At least one ends of the his-tag can reside close to the outer surface of the liposome. In an embodiment, at least one end of the polyhistidine tag is covalently attached to a MHC-I restricted peptide. The number of histidines in the polyhistidine-tag in the monolayer or bilayer can be from 1 to 6. For example, the number of histidines in the polyhistidine-tag can be 1, 2, 3, 4, 5 or 6. It is preferred to have histidines less than 6 because fewer than 6 histidines were found to provide better enhancement of immune response. In an embodiment, the poly-His tag can contain 2-5 histidines. In one embodiment, one end of the His-tag is free and a peptide is attached to the other end. It is considered that at least a part of the his-tag is located within the bilayer such that it is coordinated to the cobalt metal.

In an embodiment, the present disclosure provides antigenic compositions comprising liposomes carrying MHC-I restricted peptides, such as human MHC-I restricted peptides. The present liposomes may also comprise one or more adjuvants. Examples of adjuvants include attenuated lipid A derivatives such as monophosphoryl lipid A (MPLA), or synthetic derivatives such as 3-deacylated monophosphoryl lipid A, or Monophosphoryl Hexa-acyl Lipid A, 3-Deacyl. In various embodiments, the adjuvants may be monophosphoryl lipid A (MPLA), aluminum phosphate, aluminum hydroxide, alum, phosphorylated hexaacyl disaccharide (PHAD), Sigma adjuvant system (SAS), AddaVax (Invitrogen), or saponinQS21, CpG oligodeoxynucleotides (CpG ODN) or Polyinosinic:polycytidylic acid (poly (I:C)). In some embodiments, the adjuvant is QS21. In some embodiments, the adjuvant is PHAD. In some embodiments, the adjuvant is QS21 and PHAD. It is preferable that QS21 and PHAD are incorporated into the same liposome into which is incorporated the his-tagged MHC-I restricted peptide. QS-21 has two hydrophilic head groups with several sugar residues, and a hydrophobic region made of a triterpene group and an alkyl ester and also incorporates into bilayers. QS-21 binds to cholesterol irreversibly to form a complex, so can also be localized in the lipid bilayer. MPLA is a phosphasphoryl lipid, it may be incorporated in the liposome bilayer.

An adjuvant can be used as a 0.001 to 50 wt % solution in phosphate buffered saline, and the antigen is present in the order of micrograms to milligrams, such as about 0.0001 to about 5 wt %, such as about 0.0001 to about 1 wt %, or such as about 0.0001 to about 0.05 wt %, relative to the total mass of the peptide and lipid in the formulation. The antigen can be present in an amount in the order of micrograms to milligrams, or, about 0.001 to about 20 wt %, such as about 0.01 to about 10 wt %, or about 0.05 to about 5 wt %.

For example, a suitable mass ratio of [CoPoP:PHAD:QS21:peptide] may be 4:4:4:1. Without intending to be bound by any particular theory, it is considered that the lower percentage of adjuvant PHAD and QS21 in the liposome formulation increases the T cell responses, likely owing to too high a dose of QS-21. For example, when the peptide dose is kept the same, decreasing the percentage of MPLA and QS21 to mass ratio of [CoPoP:PHAD:QS21:peptide] equals 4:4:1:1, and the T cell responses increases. A mass ratio for [CoPoP:PHAD:QS21:peptide] equal to 4:1:1:1 is also highly immunogenic. However, without QS21 in the liposome, there is almost no CD8⁺ T cell response. Without MPLA in the formulation, the immunogenicity of the vaccine platform also decreases dramatically.

In various embodiments, ranges of CoPoP to peptide molar ratios can vary from 0.5:1 to 4:1, ranges of CoPoP:PHAD can vary from 1:1 to 1:0.1, ranges of CoPoP:QS21 can vary from 1:1 to 0.1:1.

The porphyrin group of the cobalt-porphyrin or cobalt-porphyrin conjugate making up at least part of some of the bilayer of the liposomes or other structures comprise porphyrins, porphyrin derivatives, porphyrin analogs, or combinations thereof. Exemplary porphyrins include hematoporphyrin, protoporphyrin, and tetraphenylporphyrin. Exemplary porphyrin derivatives include, but are not limited to, pyropheophorbides, bacteriochlorophylls, Chlorophyll A, benzoporphyrin derivatives, tetrahydroxyphenyl chlorins, purpurins, benzochlorins, naphthochlorins, verdins, rhodins, keto chlorins, azachlorins, bacteriochlorins, tolyporphyrins, and benzobacteriochlorins. Additional exemplary porphyrin analogs include expanded porphyrin family members (such as texaphyrins, sapphyrins and hexaphyrins) and porphyrin isomers (such as porphycenes, inverted porphyrins, phthalocyanines, and naphthalocyanines). For example, the cobalt-porphyrin can be a vitamin B₁₂ (cobalamin) or derivative thereof.

In one embodiment, the CoPoP is pyropheophorbide-phospholipid. The structure of pyropheophorbide-phospholipid is shown below:

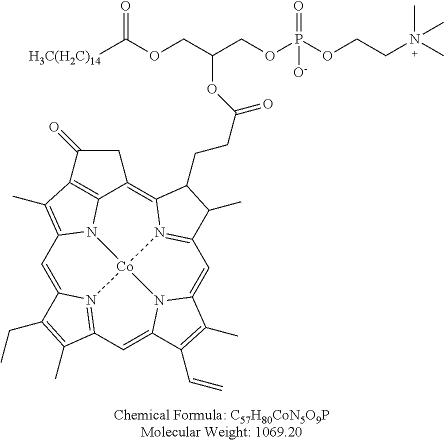

In an embodiment, the layer (monolayer or bilayer) has only CoPoP and the layer has His-tagged presentation molecules embedded therein. In this embodiment, the only phospholipid in the layer is CoPoP (i.e., CoPoP is 100 mol %). In one embodiment, the layer (monolayer or bilayer) has only CoPoP and porphyrin conjugated phospholipids (PoP), wherein CoPoP has histidines chelated thereto, with the histidines having a peptide or other presentation molecules attached thereto. In certain embodiments, there are no other phospholipids aside from CoPoP, but the layer (monolayer or bilayer) may optionally contain sterols and/or PEG-lipid.

In an embodiment, in addition to the CoPoP, the bilayer also has phospholipids which are not conjugated to porphyrin and therefore, not coordinated with Co. Such phospholipids may be referred to herein as “additional phospholipids”. In an embodiment, the only metal-PoP in the bilayer is CoPoP, which has His-tagged MHC-I restricted peptides embedded therein.

In an embodiment, the bilayer of the liposomes comprises CoPoP and PoP. In addition to the CoPoP and the PoP, the bilayer can have additional phospholipids. The bilayer may further comprise one or more sterols. In an embodiment, the bilayer consists essentially of, or consists of CoPoP, PoP, additional phospholipids, and optionally one or more sterols, and other lipids, such as gangliosides. In one embodiment, the only metal in the bilayer is Co.

In an embodiment, the CoPoP is present in the nanoparticles from 0.1 to 10 mol % with the remaining 99.9 to 90 mol % being additional lipids, with the mole percent being relative to the total amount of lipids in the bilayer. For example, the combination of CoPoP can be present from 0.1 to 10 mol %, sterol can be present from 0.1 to 50 mol %, optionally, attenuated lipid A derivatives such as monophosphoryl lipid A or 3-deacylated monophosphoryl lipid A or a related analog can be present from 0 to 20 mol % or 0.1 to 20 mol %, and the remainder is additional phospholipids. Examples of additional phospholipids include DOPC, DSPC, DMPC, or combinations thereof. The sterol, if present, can be cholesterol.

In an embodiment, the combination of CoPoP and PoP may be present in the nanoparticles from 0.1 to 10 mol % with the remaining 99.9 to 90 mol % being additional phospholipids. For example, the combination of CoPoP and PoP can be present from 0.1 to 10 mol %, sterol can be present from 0 to 50 mol % or 0.1 to 50 mol %, PEG can be present from 0 to 20 mol % or 0.1 to 20 mol %, and the remainder is additional phospholipids. The additional phospholipids can be DOPC, DSPC, DMPC, or combinations thereof. The sterol, if present, can be cholesterol.

In various embodiments, in addition to the porphyrin conjugates disclosed herein, the bilayer of the liposomes also comprises other phospholipids. The fatty acid chains of these phospholipids may contain a suitable number of carbon atoms to form a bilayer. For example, the fatty acid chain may contain 12, 14, 16, 18, or 20 carbon atoms. In different embodiments the bilayer comprises phosphatidylcholine, phosphatidylethanoloamine, phosphatidylserine and/or phosphatidylinositol.

The present bilayers and monolayers may also comprise sterols. The sterols may be animal sterols or plant sterols. Examples of sterols include cholesterol, sitosterol, stigmasterol, and cholesterol. In embodiments, cholesterol may be from 0 mol % to 50 mol %, or 0.1 to 50 mol %. In other embodiments, cholesterol may be present from 1 to 50 mol %, 5 to 45 mol %, 10 to 30 mol %.

In certain embodiments, the bilayer or monolayer further comprises an adjuvant such as attenuated lipid A derivatives such as monophosphoryl lipid A or 3-deacylated monophosphoryl lipid A.

The liposomes may be spherical or non-spherical. The size (e.g., longest linear dimension) of the liposomes can be from 50 to 1000 nm or more. In an embodiment, the liposomes have a size (e.g., a longest dimension such as, for example, a diameter) of 50 to 1000 nm, including all integer nm values and ranges therebetween. For example, the size may be from 50 to 200 nm or from 20 to 1000 nm. If the liposomes are not spherical, the longest dimension can be from 50 to 1000 nm. These dimensions can be achieved while preserving the nanostructure width of the bilayer. The RBD sequence or portion thereof can be incorporated in the bilayer. The liposomes can additionally carry cargo in the aqueous compartment. In an embodiment, the liposomes can have a size of 30 nm to 250 nm, including all integers to the nm and ranges therebetween. In an embodiment, the size of the liposomes is from 100-175 nm. In an embodiment, at least 50%, 60%, 70%, 80%, 85%, 90%, 95%, 99%, or 100% of the liposomes in the composition have a size of from 30 to 250 nm or from 100 to 175 nm. The liposomes can be more than 200 nm. In an embodiment, the liposomes are more than 1000 nm. In an embodiment, the nanostructures are from 200 to 1000 nm. In an embodiment, the largest dimensions of the liposomes are less than 200 nm, while preserving the nanostructure width of the bilayer. In an embodiment, the size of the liposomes exceed 200 nm in some dimensions, while preserving the nanostructure width of the bilayer. In an embodiment, the size of the liposomes exceed 1000 nm in some dimensions, while preserving the nanostructure width of the bilayer.

In one aspect, the disclosure provides a composition comprising liposomes or other structures of the present disclosure or a mixture of different liposomes or other structures. The compositions can also comprise a sterile, suitable carrier for administration to individuals including humans, such as, for example, a physiological buffer such as sucrose, dextrose, saline, pH buffering (such as from pH 5 to 9, from pH 7 to 8, from pH 7.2 to 7.6, (e.g., 7.4)) element such as histidine, citrate, or phosphate. In one embodiment, the composition comprises at least 0.1% (w/v) CoPoP liposomes or His-tagged-CoPoP liposomes or other structures. In various embodiments, the composition comprises from 0.1 to 100 mol % CoPoP liposomes or His-tagged CoPoP liposomes or other structures such as bilayer coated nanoparticles. In one embodiment, the composition comprises from 0.1 to 99 mol % CoPoP liposomes having His-tagged presentation molecules associated therewith.

In one embodiment, the compositions of the present disclosure are free of maleimide or succinimidyl ester reactive groups. In one embodiment, the tagged molecule to be attached to the membrane does not have a non-natural amino acid.

The MHC-I restricted peptides bearing the His-tag may be peptide containing from 4 to 11 amino acids. For example the peptides may have 4, 5, 6, 7, 8, 9, 10 or 11 amino acids (excluding the histidines). In various embodiments, the peptides may have 5 to 11, 5 to 10, 6 to 11, 6 to 10, 7 to 11, 7 to 10, 8 to 11, 8 to 10, 9 to 11, 9 to 10 amino acids (excluding the histidines). Antigen selection is a key step of developing a peptide cancer vaccine. Examples of human peptides include Melan A/MART₁₂₆₋₃₅ (EAAGIGILTV (SEQ ID NO:30)), Melan A/MART₁₂₇₋₃₅ (AAGIGILTV (SEQ ID NO:31)), Tyrosinasei-9 (MLLAVLYCL (SEQ ID NO:32)), Tyrosinase₃₆₈₋₃₇₆ (YMDGTMSQV (SEQ ID NO:33)), Gp100₄₅₇₋₄₆₆ (LLDGTATLRL (SEQ ID NO:34)), Survivin-2B₈₀₋₈₈ (AYACNTSTL (SEQ ID NO:35)), NY-ESO-1b₁₅₇₋₁₆₅ (SLLMWITQC (SEQ ID NO:36)), WT1₂₃₅₋₂₄₃ (mp235) (CMTWNQMNL (SEQ ID NO:37)), gp100₂₀₉₋₂₁₇ (210M) (IMDQVPFSV (SEQ ID NO:38)), gp100₂₈₀₋₂₈₈ (288V) (YLEPGPVTA (SEQ ID NO:39)), E7₁₁₋₂₀ (YMLDLQPETT (SEQ ID NO:40)), E7₈₆₋₉₃ (TLGIVCPI (SEQ ID NO:41)) and E7₈₂₋₉₀ (LLMGTLGIV (SEQ ID NO:42)). The peptides can have only naturally occurring amino acids, or can be a mixture of naturally occurring and non-naturally occurring amino acids, or can have only non-naturally occurring amino acids. Peptide for human vaccine can be commercially customized. The structures formed by the layers of the present disclosure are serum stable. For example, in vitro, the his-tag binding stability to the CoPoP bilayers is stable when incubated in 40% human serum at 37° C. for 21 days. Thus, these structures can be stable under serum or concentrated or diluted serum conditions.

In some embodiments, a composition suitable for treatment in humans could comprise 0.05 to 1 mg peptide, including all 0.01 mg values and ranges therebetween; 0.05 to 4 mg CoPoP, including all all 0.01 mg values and ranges therebetween; 0.01 to 0.5 mg QS21 including all 0.01 mg values and ranges therebetween; and 0.01 to 1 mg MPLA, including all 0.1 mg values and ranges therebetween.

The present disclosure also provides methods for using structures bearing the bilayers as described herein. In one embodiment, this disclosure provides a method of eliciting an immune response in a host. The immune response may generate CD8⁺ T cells. The method comprises administering to an individual a composition comprising a structure bearing CoPoP bilayers to which is conjugated one or more histidine tagged MHC-I restricted peptide or peptides. The compositions may be administered by any standard route of immunization including subcutaneous, intradermal, intramuscular, intratumoral, or any other route. The compositions may be administered in a single administration or may be administered in multiple administrations including booster shots. T cells and cytokine production can be measured to monitor the immune response.

In an embodiment, the present liposomes and compositions may be used in conjunction with other anti-cancer therapies. For example, the treatment using the present compositions may be augmented by checkpoint blockade such as PD-1, PD-L1, or CTLA-4 antibodies.

In one aspect, the disclosure provides a method of preparing bilayers comprising CoPoPs. Freebase PoP can be produced by esterifying a monocarboxylic acid porphyrin such as pyropheophorbide-a with 2-palmitoyl-2-hydroxy-sn-glycero-3-phosphocholine (lyso-C16-PC), (Avanti #855675P) using 1-Ethyl-3-(3-dimethylaminopropyl) carbodiimide and 4-dimethylaminopyridine in chloroform at a 1:1:2:2 lyso-C16-PC:Pyro:1-ethyl-3-(3-dimethylaminopropyl)carbodiimide (EDC):4-dimethylaminopyridine (DMAP) molar ratio by stirring overnight at room temperature. The PoP is then purified by silica gel chromatography. CoPoP can be generated by contacting porphyrin-phospholipid conjugate with a molar excess (e.g., 10-fold molar excess) of a cobalt salt (e.g., cobalt (II) acetate tetrahydrate) in a solvent (e.g., methanol) in the dark.

The following composition and method examples illustrate various aspects of the present disclosure:

A liposome comprising a) a bilayer, wherein the bilayer comprises: i) one or more phospholipids, and ii) one or more porphyrin-phospholipid conjugates having cobalt coordinated thereto forming cobalt-porphyrin phospholipid conjugate; and b) a polyhistidine-tagged MHC-I restricted peptide, wherein at least a portion of the polyhistidine tag resides in the hydrophobic portion of the bilayer and one or more histidines of the polyhistidine tag are coordinated to the cobalt in the cobalt-porphyrin, wherein at least a portion of the polyhistidine-tagged MHC-I restricted peptide is exposed to the outside of the liposome, the liposome will bind to MHC-I class of molecules, but not MHC-II class of molecules, the polyhistidine-tag comprises 2-6 histidine residues, and the MHC-I restricted peptide is a peptide of from 4 to 11 amino acids, said number of amino acids not including the histidines of the His-tag. In various embodiments, the polyhistidine-tag comprises 2-5 histidine residues, the MHC-I restricted peptides does not bind to MHC-II class molecules, the liposome may further comprises one or more additional adjuvants incorporated therein, which may be QS21 or PHAD, the one or more additional adjuvant may further comprises MPLA (including synthetic variants such as PHAD, 3D6A-PHAD, or 3D-PHAD), the one or more additional adjuvant may further comprise MPLA and QS21, the mass ratio of the MHC-I restricted peptide to the QS21 may be from 1:1 to 10:1, the mass ratio of the MHC-I restricted peptide to the PHAD may be from 1:1 to 1:10, the MHC-I restricted peptide, the QS21 and PHAD may be present in mass ratios of 1:1:1, 2:1:1, 3:1:1, 4:5:1:1, 6:1:1, 7:1:1, 8:1:1, 9:1:1, or 10:1:1, the cobalt porphyrin may be conjugated to a phospholipid to form a cobalt porphyrin-phospholipid conjugate, the cobalt porphyrin-phospholipid conjugate may make up from 1 to 25 mol % of the monolayer or the bilayer, the cobalt porphyrin-phospholipid conjugate may make up from about 2% to about 8%, more specifically 3-4% (mass ratio) of the bilayer, the bilayer may further comprise cholesterol with mass ratio of about 15 to 20% (e.g., 17-19%), and/or the size of the liposome may be 50 nm to 250 nm.

A method for generating an immune response against a tumor antigen, reducing the growth of a tumor in a subject, and/or inducing CD8+ positive cells in a host individual against tumor cells comprising administering to the individual a composition comprising the liposomes of as described herein in a pharmaceutical carrier. In embodiments, the individual is a human or non-human animal.

The following examples are presented to illustrate the present disclosure. They are not intended to limiting in any manner.

### Example 1

This example describes preparation and use of liposomes comprising MHC-I restricted peptides. This example describes studies in mice, where immunization with a short MHC-I peptide derived from the gp70 oncogene as a model system generated functional, Ag-specific CD8⁺ T cells, resulting in rejection of multiple tumor cell lines, durable immunity, and control of local and metastatic disease. In vivo screening of peptide micro-libraries comprising a hundred putative MHC-I binding cancer epitopes revealed that a handful were immunogenic. One epitope in the Rrgac gene shared by both mammary and colon cancer cell lines reduced metastatic disease following immunization. To identify relevant human peptides, one or more neo-epitopes predicted to be MHC-I binders (using tumor cells or cell lines) may be incorporated into the present liposomes and their ability for generating or enhancing a functional immune response (e.g., inhibiting tumor growth) may be evaluated. The method was as follows. We mixed the top 20 neo-epitopes (instead of top100) that predicted to be the best MHC-I binder from RENCA tumor cells with CPQ liposome, the vaccine inhibited tumor growth and (AYTTQREEL (SEQ ID NO:43)) were screened as the functional neo-antigen. HPV-16 cellular oncoproteins E6 and E7 were screened with the same method, 6 peptides that predicted to be the best MHC-I binder admixed with CPQ liposome, the vaccine inhibited tumor growth and the previously identified E7₄₉₋₅₇ epitope was screened ad the functional epitope.

Results

Short Peptides Form Particles when Admixed with CoPoP/PHAD/QS-21 (CPQ) Liposomes

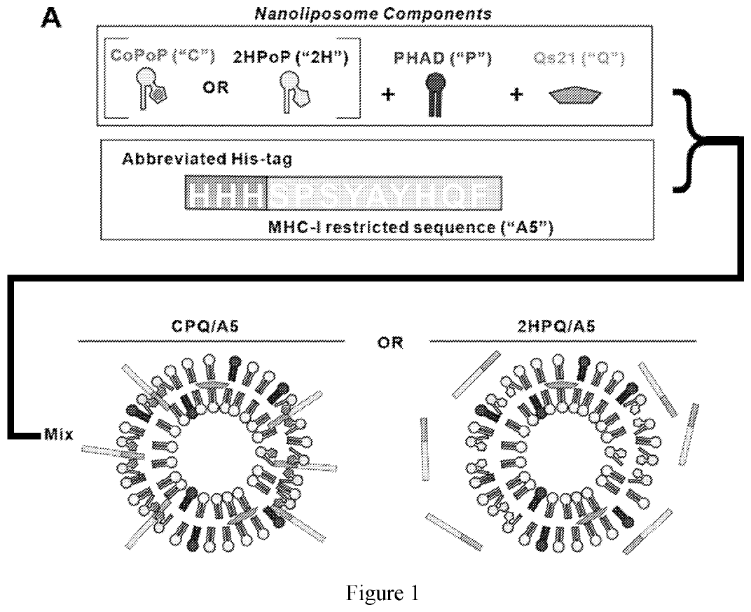

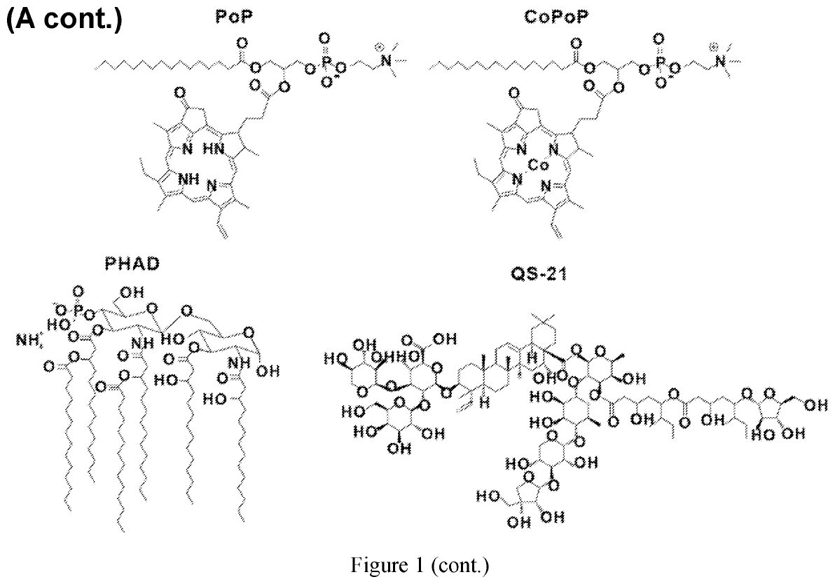

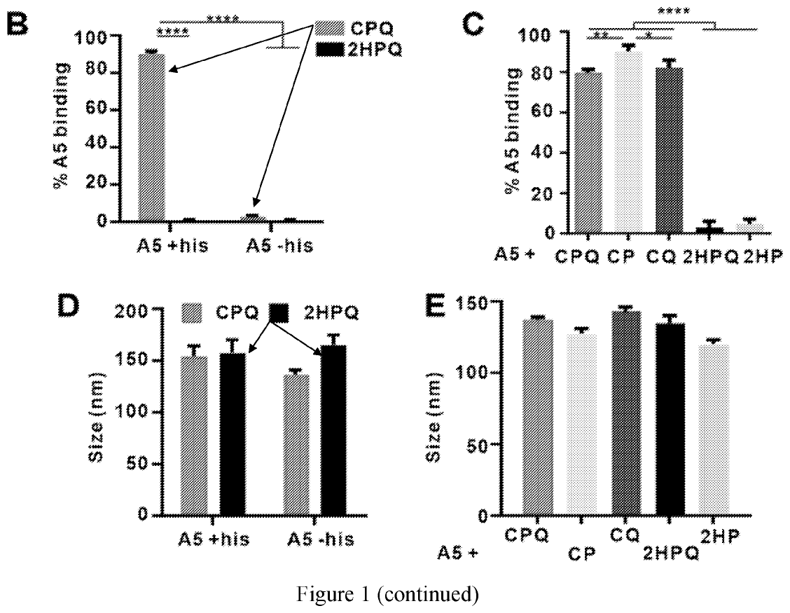

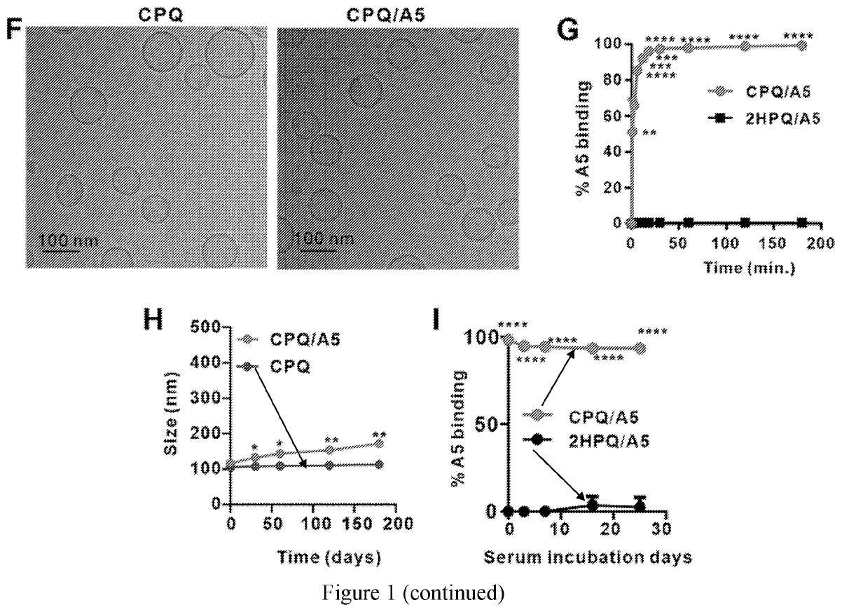

As shown in FIG. 1A, liposomes were formed with CoPoP, along with the immunostimulatory adjuvants QS-21, a saponin, and PHAD, a synthetic monophosphoryl lipid A (MPLA). The chemical structure of porphyrin-phospholipid (PoP, or 2HPoP since two hydrogens are present instead of cobalt), CoPoP, PHAD and QS-21 are shown in FIG. 1A. With 3 active components in the bilayer, these are referred to as “CPQ”. A role of CoPoP is to induce particle formation of the MHC-I restricted peptides. QS-21 and monophosphoryl lipid A (MPLA) are components of AS01, a liposomal adjuvant used in licensed vaccines for malaria and herpes zoster. MPLA is a Toll-like receptor 4 agonist lipid that can incorporate into bilayers. QS-21 has two hydrophilic head groups with several sugar residues, and a hydrophobic region made of a triterpene group and an alkyl ester and also incorporates into bilayers. QS-21 binds to cholesterol irreversibly to form a complex, so can also be localized in the lipid bilayer. Alone, it can bind to local cellular cholesterol and causes necrosis at injection sites. It can also bind to cholesterol in the lipid bilayer of erythrocytes and causes pores. To reduce QS-21 hemolysis and toxicity of, it is formulated with liposomes that contain cholesterol. Throughout this study, control liposomes that are identical to CPQ, but where two hydrogen atoms replace the cobalt in the porphyrin macrocycle are used as non-particle forming control liposomes and are termed “2HPQ”. Thus, comparison of CPQ and 2HPQ adjuvants enables examination of the impact of particle-based presentation of short peptides on immunogenicity.

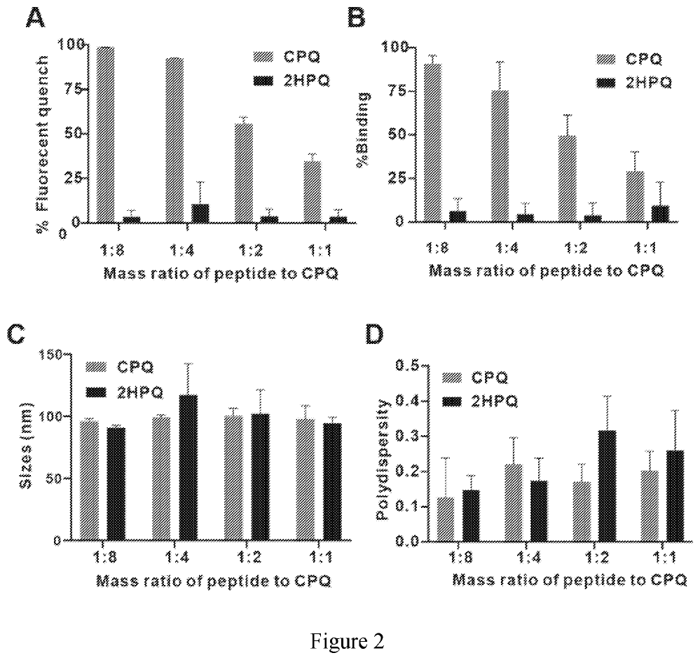

The A5 peptide was found to only bind with CPQ liposome when it contained a his-tag (FIG. 1B). Shortly after mixing A5 with liposomes, ˜80% were converted into particle form, as assessed by a microcentrifugal filtration assay. QS-21 and PHAD did not impact the binding between the liposome and peptide (FIG. 1C). However, the corresponding liposomes that contained the porphyrin but lacking cobalt displayed minimal binding to A5. Following particle formation with the peptide, the size of all the liposomes (CPQ, 2HPQ, CoPoP/PHAD (“CP”), CoPoP/QS-21 (“CQ”), and 2HPoP/PHAD (“2HP”)) remained ˜100-150 nm based on dynamic light scattering (FIG. 1D and FIG. 1E). Cryo-electron microscopy revealed that both CPQ liposomes with or without peptide bound were spherical and unilamellar, with size close to 100 nm (FIG. 1F). To test peptide binding kinetics and serum stability of the resulting vaccine, A5 was fluorescently labeled. Upon liposome binding, fluorescence energy transfer from the fluorophore to CPQ or 2HIPQ liposomes results in energy transfer, which can be measured. Within 20 min of incubation, the labeled A5 peptide fluorescence was fully quenched, reflecting rapid binding to the liposomes (FIG. 1G). 2HIPQ did not quench any fluorescence of A5 peptide. For maximum loading efficacy of the peptide, approximately 4 times the mass ratio of CoPoP was used (FIG. 2). These results are consistent with the microcentrifugal filtration assay using the unlabeled peptide. In refrigerated storage, CPQ with or without the A5 peptide bound was stable for at least three months, with sizes from 100-150 nm (FIG. 1H). In 40% human serum, the binding of A5 and CPQ liposomes remained intact for weeks, reflecting peptide-particle formation that is highly stable in biological fluids (FIG. 1I).

A5/CPQ Liposome Induces Robust Ag-Specific CD8+ T Cell Responses

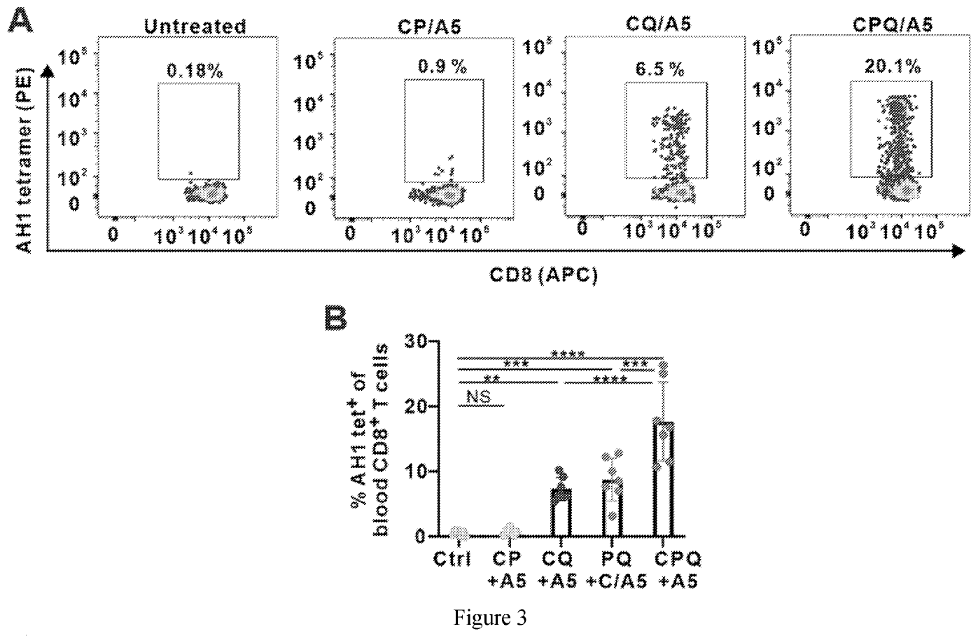

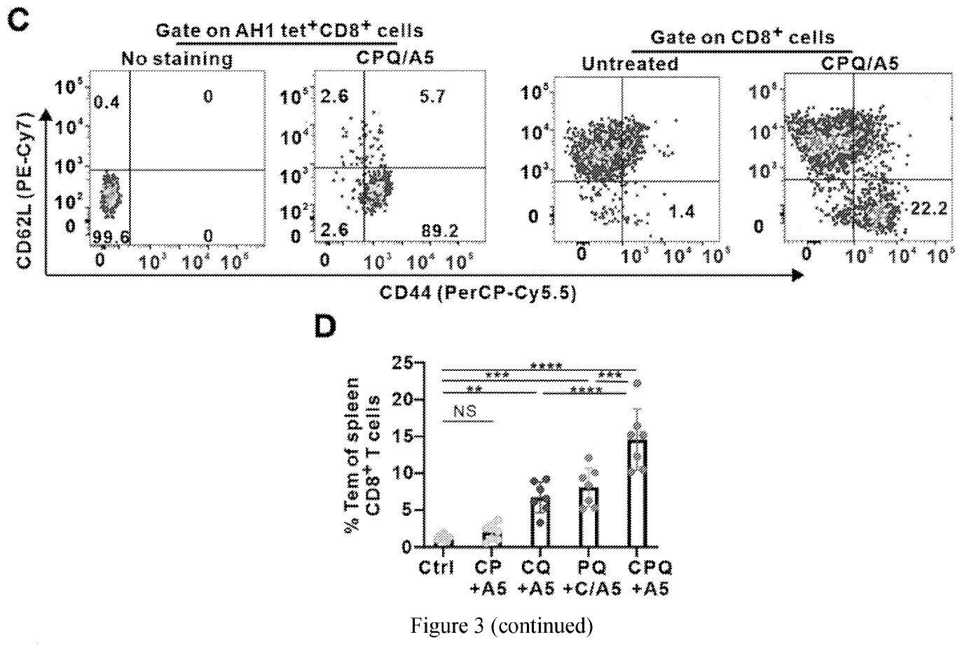

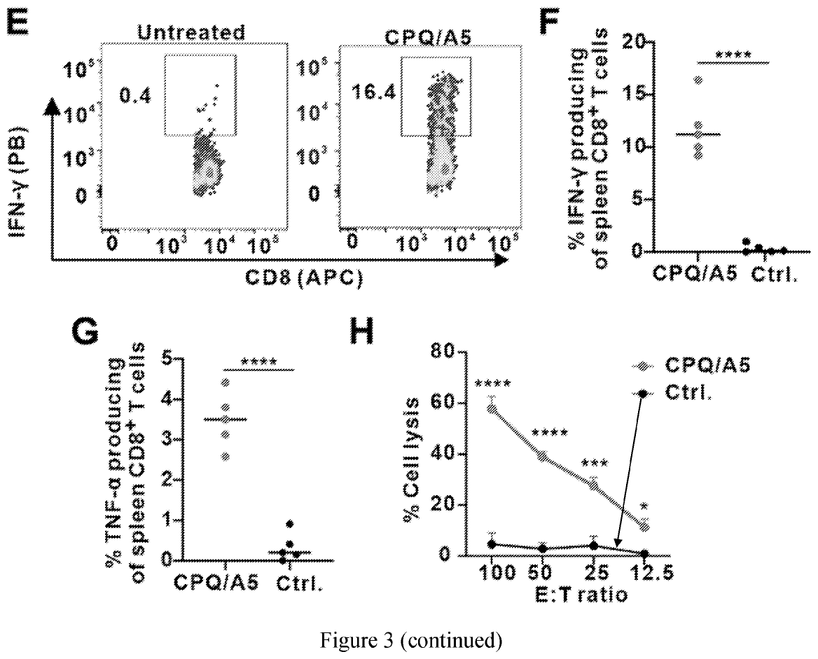

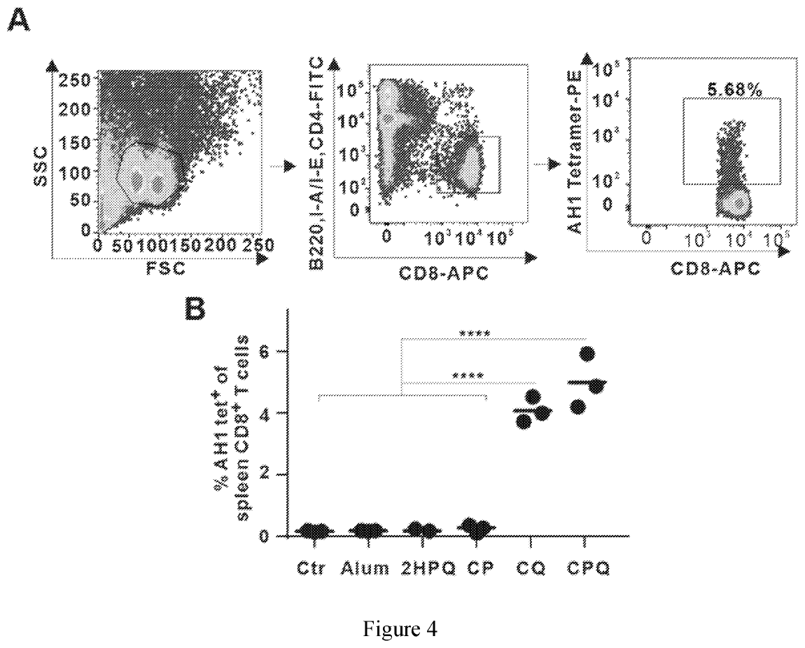

Next, BALB/c mice were immunized with 500 ng A5, admixed with CPQ on days 0 and 7, and peripheral blood was collected on day 7 and day 13. Mice immunized with CPQ admixed with A5 (CPQ/A5) induced ˜20% Ag-specific cells within the CD8⁺ T cell population. Vaccination using CoPoP liposomes without QS-21 (CP/A5) did not produce detectable Ag-specific CD8⁺ T cells in the blood (FIG. 3A and FIG. 3B) or spleen (FIG. 4). Without PHAD in the liposome, CQ/A5 produced less Ag-specific CD8⁺ T cells compared to CPQ/A5. The vast majority of these AH1 tetramer-positive CD8⁺ T cells (89%) were of the effector-memory T cell (Tem) phenotype based on differential expression of CD44 and CD62L (FIG. 3C). The overall percent of effector-memory CD8⁺ T cells was increased significantly after CPQ/A5 vaccination (FIG. 3D). We also tested whether presentation of the A5 peptides together on the same particle as QS-21 and PHAD was important. To do so, mice were immunized with CPQ/A5, or alternatively with CoPoP liposomes with A5 (C/A5) admixed with 2HIPQ liposomes, with equivalent doses of peptide and adjuvant. While particle presentation of A5 with QS-21 and PHAD in separate liposomes could still induce Ag-specific CD8⁺ T cells, presentation of all components on the same particle was significantly more effective.

Splenocytes were collected for intracellular staining of interferon-gamma (IFN-γ) and tumor-necrosis factor alpha (TNF-α) staining. Splenocytes from untreated mice or mice vaccinated with CPQ/A5 were prepared for in vitro stimulation by the A5 peptide. There was robust IFN-γ production after peptide stimulation; wherein ˜12% of the CD8⁺ T cells produced IFN-γ after stimulation and ˜4% of the CD8⁺ T cells produced TNF-α. This indicates that CPQ/A5 induced a strong CD8⁺ T cell response, as measured by cytokine production (FIG. 3E, FIG. 3F and FIG. 3G). Splenocytes were stimulated by A5 peptide and cultured together with IL2 for 5 days and then used as effector cells (E) and CT26 cells were pulsed with A5 peptide as target cells (T). These effector T cells lysed 60% of the tumor cells in 4 hours at an effector to target (E:T) cell ratio of 100 (FIG. 3H). Initial studies with the A5 peptide included a zwitterionic charged C-terminus tripeptide comprising the charged amino acids “ERR” with the aim to improve solubility, since MHC-I binding epitopes tend to be hydrophobic. However, this was later found to be non-essential and was not included for A5 in subsequent experiments. Other peptides might stand to benefit from solubilization approaches, which facilitate controlled peptide dissolution and dosing. The peptide sequences used for all experiments in this study are listed in Table 1-2.

CPQ/A5 as a Prophylactic Cancer Vaccine

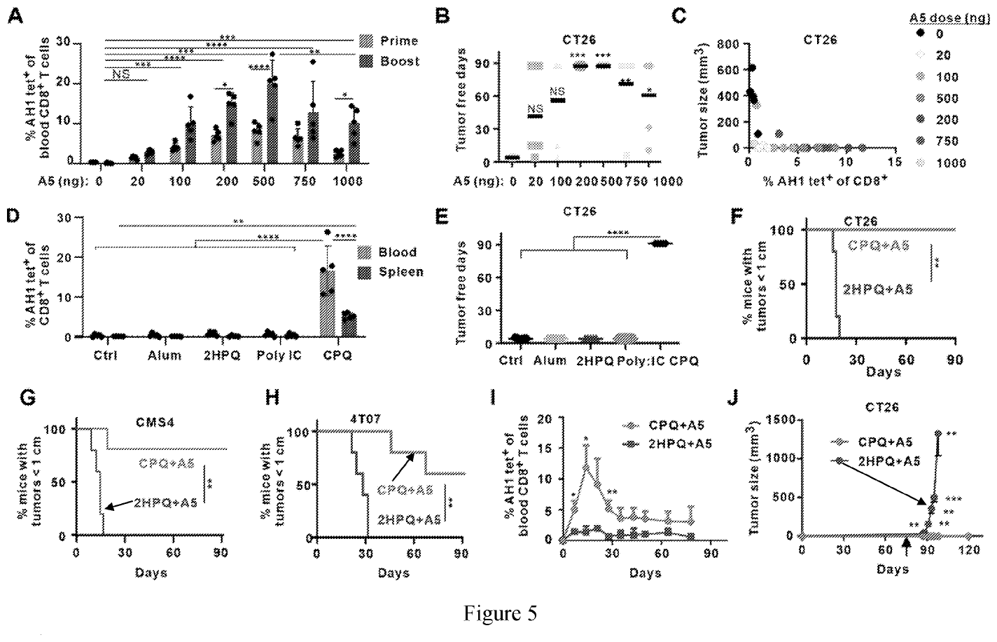

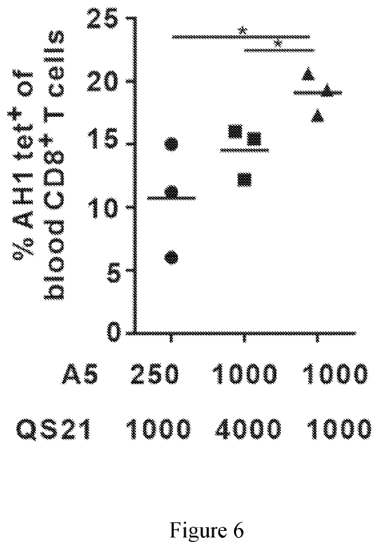

To test the function of the vaccine-induced, Ag-specific CD8⁺ T cells, BALB/c mice immunized with varying nanogram doses of CPQ/A5 mice were challenged with CT26 cells, which express gp70. 7 days after the first vaccination, there was an increase of AH1-specific CD8⁺ T cells. After the booster vaccination, even as little as 20 ng of A5 (along with 80 ng CoPoP, 80 ng PHAD and 80 ng QS-21) induced 5% of peripheral CD8⁺ T cells to be Ag-specific (FIG. 5A). A general dose response was followed, with increasing CPQ/A5 doses inducing a stronger CD8⁺ T cell response, up to a plateau of 500 ng A5. Higher doses did not increase the percentage of Ag-specific cells in CD8⁺ T cells, and actually resulted in a lower frequency. Since the peptide to adjuvant ratio was fixed in all cases, at higher peptide doses, more QS-21 was also administrated, which may have induced toxicity in the immune cells in the draining lymph nodes. Indeed, when 1000 ng A5 was immunized with liposomes formulated to provide 1000 ng QS-21 instead of 4000 ng (which was used with the fixed Ag to adjuvant ratio), Ag-specific CD8⁺ T cells increased significantly (FIG. 6).

Within four days of tumor challenge, all control mice developed palpable tumors. However, following vaccination with just 20 ng of A5 peptide, 40% of the immunized mice remained tumor-free for 90 days post-tumor challenge (FIG. 5B). Immunization with 200 or 500 ng of A5 led to complete tumor rejection in all mice. Mice immunized with doses higher than 500 ng actually had lower tumor rejection rates, although the average tumor-free period was still 70 and 60 days after tumor challenge, respectively. The lower protection rate is in accordance to the Ag-specific CD8⁺ T cell frequency data. Amongst the mice that developed tumors, higher induction of Ag-specific CD8⁺ T cells were associated with smaller tumor sizes (FIG. 5C).

Next, we compared the immunogenicity of 500 ng A5 peptide admixed with CPQ or other vaccine adjuvants including 2HIPQ (lacking cobalt), Alum, or poly(I.C). Only mice immunized with CPQ admixed with 500 ng A5 produced AH1-specific CD8⁺ T cells in both the blood and spleen (FIG. 5D), which was accompanied by completely rejection of a CT26 tumor challenge (FIG. 5E). In contrast, mice immunized with 500 ng A5 admixed with other adjuvants did not produce detectable AH1-specific CD8⁺ T cells and developed tumors within four days of challenge. Thus, CPQ improved immunization of the short A5 peptide.

Although gp70 is a shared biomarker expressed in several murine cancer cell lines, its use in cancer vaccines has generally been focused on CT26 tumors. To assess whether the CPQ/A5 could offer protection in other models, mice immunized with CPQ/A5 and non-identical but non-particle forming 2HPQ/A5 control were challenged with CT26 cells (FIG. 5F), as well as other models that express gp70; the CMS4 murine sarcoma (FIG. 5G) and the orthotopic 4T07 breast cancer (FIG. 5H) model. Immunization with CPQ/A5 significantly prevented tumor growth, resulting in much lower percentages of mice with tumor sizes reaching 1 cm following tumor challenge in all three cancer models, with 60-100% of mice showing complete tumor rejection.

The durability of CPQ/A5 immunization was next assessed. Mice were immunized with 500 ng A5 admixed with CPQ or 2HIPQ on days 0 and 7, and the Ag-specific CD8⁺ T cell response was followed in the peripheral blood. Ag-specific CD8⁺ T cells increased following the initial immunization and boosting, with a maximum frequency observed on day 14 (FIG. 5I). Mice immunized with 2HPQ/A5 had minimal Ag-specific CD8⁺ T cells at all time points tested. After day 14, the Ag-specific CD8⁺ T cell population gradually subsided, but was sustained around 5% of all CD8⁺ T cells by day 80. On day 80, over two months after the final boosting with 500 ng A5, mice were challenged with CT26 cancer cells. As shown in FIG. 5J, even at this time point, all CPQ/A5 immunized mice fully rejected the tumor challenge without any sign of growth for at least 40 days. All mice vaccinated with A5 admixed with cobalt-free liposomes developed rapidly growing tumors.

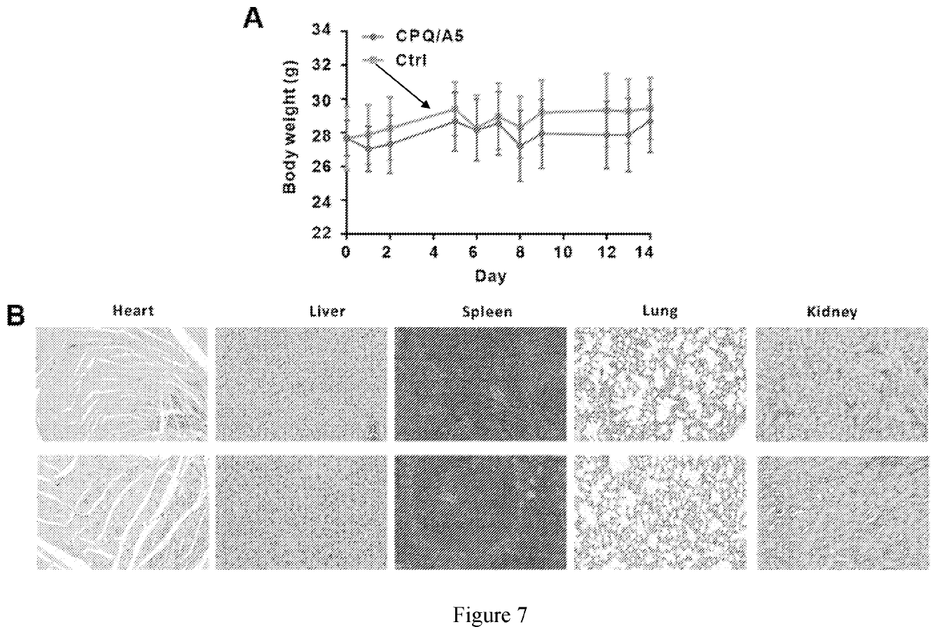

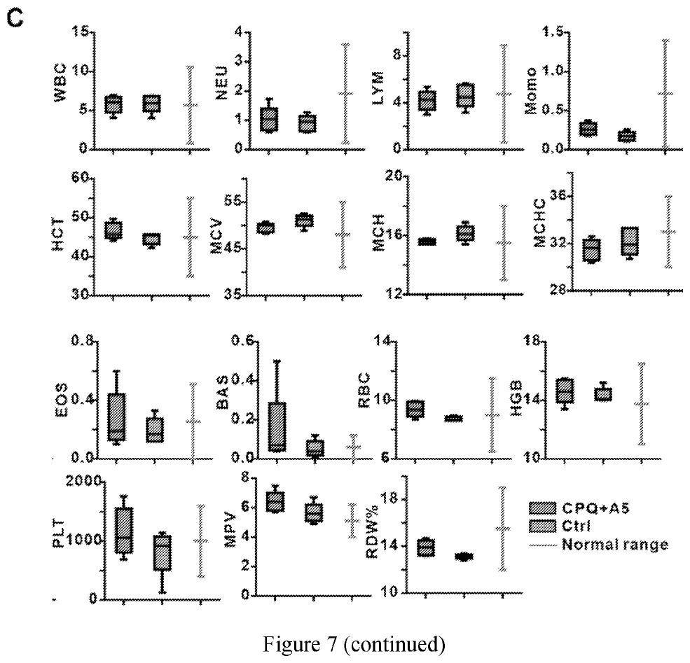

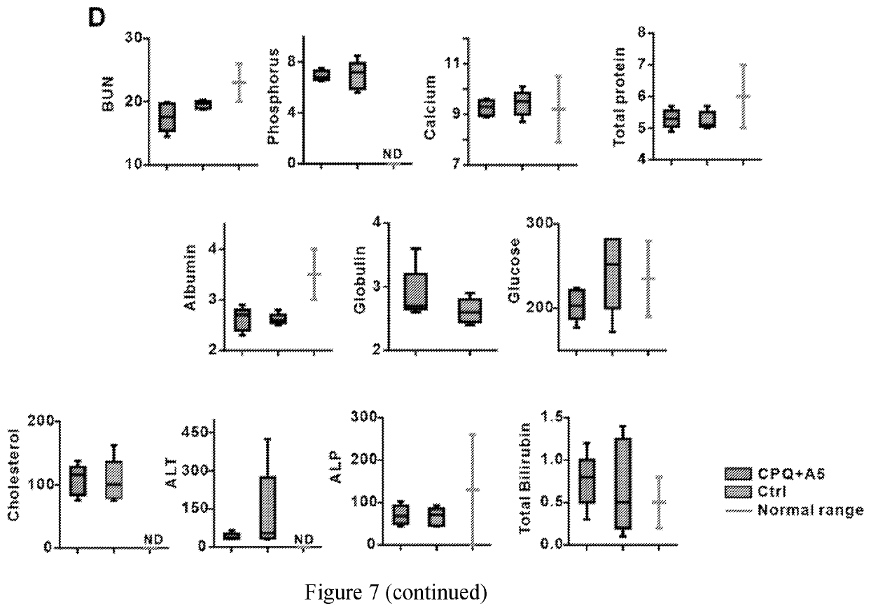

Safety studies were carried out in CD-1 mice, which were primed on day 0 and boosted on day 7 with CPQ/A5 at the functional dose of 500 ng of peptide. Outbred mice were selected to get a broader representation of potential toxicity responses. Mice exhibited normal weight gain (FIG. 7A). No obvious differences in the heart, liver, spleen, lung or kidney of mice were observed with histology (FIG. 7B). A complete blood cell count (FIG. 7C) and serum chemistry panel (FIG. 7D) revealed no significant differences between healthy mice and vaccinated mice.

CPQ/A5 as a Therapeutic Cancer Vaccine

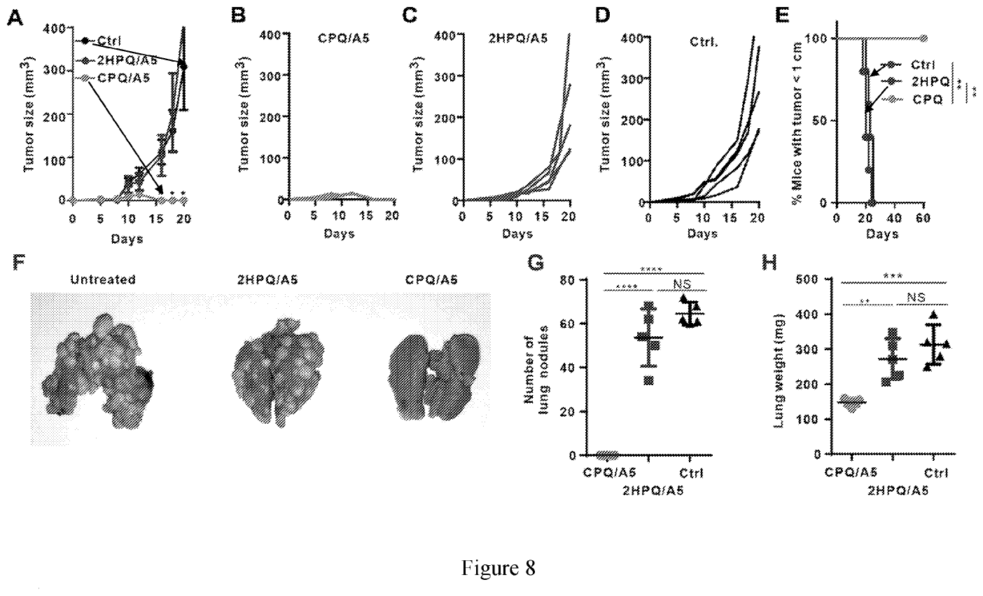

While CPQ/A5 was shown to be potent in a prophylactic setting, most cancer vaccines would be initially tested in patients with advanced or metastatic disease. To address this, CPQ/A5 was assessed in mice after tumor implantation or in settings of experimental lung metastasis. In the former setting, mice were inoculated with CT26 tumors 5 days before immunization. Mice were then immunized on day 5 with 500 ng A5, a time point at which tumors first became measurable and started rapidly growing (FIG. 8A). Mice were boosted with 500 ng A5 a week later, and in the intervening period, tumors grew to ˜3 by 3 mm by day 12. However, within days following the second immunization with CPQ/A5, all tumors shrank and disappeared without evidence of regrowth for 90 days, with 100% survival (FIG. 8B). Control mice or mice that were immunized with non-particle forming 2HPQ/A5 exhibited rapid tumor growth and no mice survived (FIG. 8C, FIG. 8D and FIG. 8E).

To test CPQ immunization in a metastatic setting, an experimental CT26 lung metastasis model was established by intravenous injection of tumors cells. Two and 9 days later, mice were immunized with 500 ng A5. Eighteen days after cancer cell injection, lung nodules were recorded. In untreated mice or in mice receiving the non-particleizing 2HPQ/A5 vaccine, dozens of nodules were observed (FIG. 8F). In stark contrast, nodules were not detectable at all in mice that were immunized with CPQ/A5. Control mice or mice injected with 2HPQ/A5 had on average more than 50 lung nodules per mouse (FIG. 8G). Mice immunized with CPQ/A5 did not have any visible evidence of disease. Lung metastasis was confirmed by increased lung weights; mice without any treatment or injected with 2HPQ/A5 had nearly double the lung weight compared to mice immunized with CPQ/A5 (FIG. 8H).

CPQ Mechanistic Features

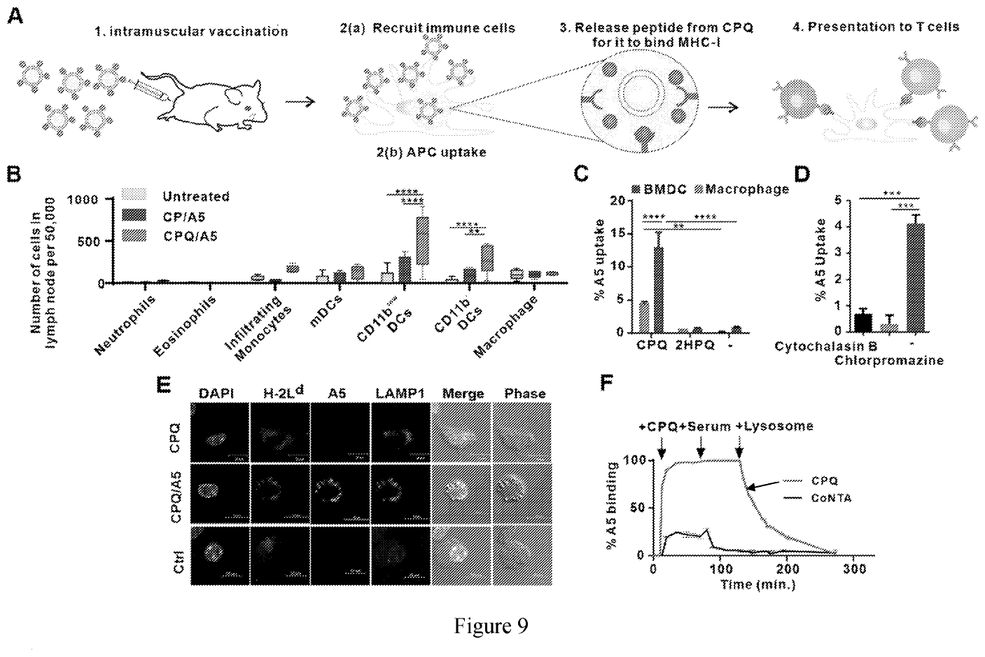

Next, we sought to determine the immunologic basis for vaccine potency of CPQ/A5. After admixing with CPQ, short peptides form particles that are stable in serum (FIG. 1), which can be transported to the draining lymph nodes. A schematic is shown in FIG. 9A. Immune cells are recruited to the lymph nodes, where Ag-presenting cells (APCs) take up the liposomes into phagosomes and endosomes. There, peptides are putatively released from the CPQ liposome where they are presented on MHC-I molecular expressed prior to externalization and activation of T cells.

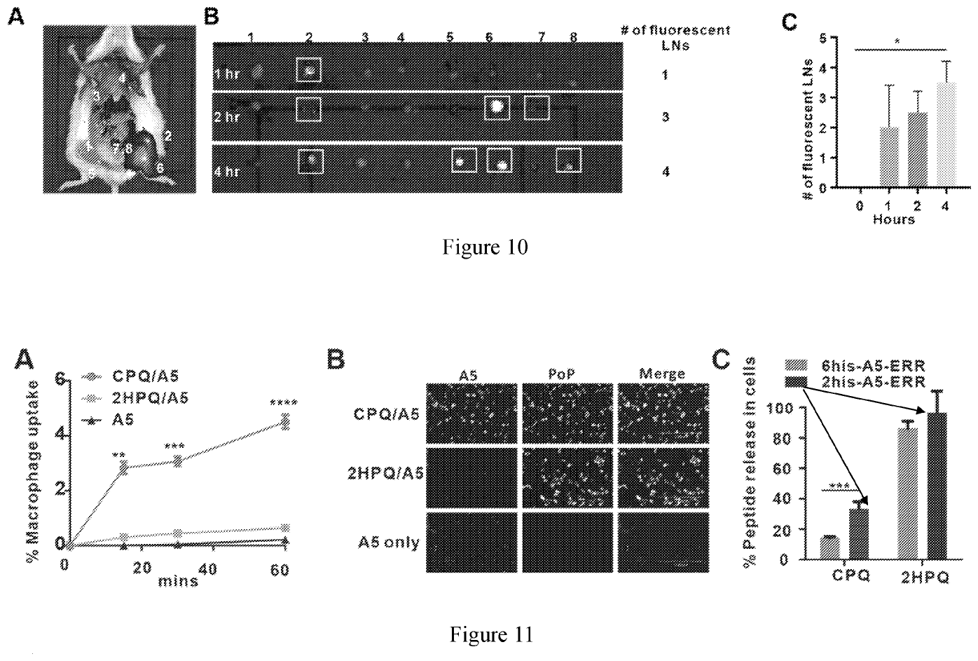

Following intramuscular administration, liposomes migrated to 1-3 lymph nodes close to the injection site in an hour and 3-4 lymph nodes in 4 hours (FIG. 10). To investigate immune cell recruitment, the draining inguinal lymph nodes were collected, and the cells were analyzed by flow cytometry 48 hours after intramuscular immunization with CPQ/A5 or CP/A5 (the difference between these being the inclusion of QS-21 in the CPQ liposomes). Compared to CP/A5-vaccinated mice and untreated mice, higher numbers of CD11b^{low} dendritic cells (DCs) and CD11b⁻ DCs were recruited in the lymph nodes of mice vaccinated with CPQ/A5 (FIG. 9B). MPLA and QS-21 has been shown to facilitate immune cell recruitment to the injection site. Other immune cell types (neutrophils, eosinophils, infiltrating monocytes, myeloid DCs (mDCs) and macrophage) were not observed to significantly increase in draining lymph nodes.

The modest increase of certain immune cells in the lymph nodes following immunization does not likely fully account for the robust enhancement in the Ag-specific CD8⁺ T cell response by CPQ. The uptake of the peptide Ag was examined in vitro using fluorescence microscopy with a labeled A5 peptide. Macrophages and bone marrow derived dendritic cells (BMDCs) were incubated with CPQ/A5 or 2HPQ/A5 and uptake was assessed. When admixed with CPQ, 5% of the total A5 peptide in the incubation mixture was taken up by macrophages, and 13% were taken up by BMDCs (FIG. 9C). However, there was no Ag uptake when A5 alone or 2HPQ/A5 were incubated with the cells. Kinetics revealed that in macrophages, ˜3% of the CPQ/A5 peptide is taken up within 20 minutes, increasing to 4-5% in an hour (FIG. 11A). Fluorescence microscopy confirmed these results; A5 was only taken up in macrophages when peptides were admixed with CPQ liposomes (FIG. 11B). Interestingly, as CPQ quenches the fluorescence of the labeled A5 peptide upon binding, the strong fluorescence of labeled A5 in the micrographs suggest that the peptide may be released from the liposomes after cellular uptake. Release of the peptide was detected in cells based on a fluorometric quenching assay that suggested there was greater intracellular peptide release with a shorter his-tag length (FIG. 11C). In the presence of cytochalasin B (a phagocytosis inhibitor) and chlorpromazine (an inhibitor of clathrin-mediated endocytosis), significantly less A5 peptide was taken up by macrophages (FIG. 9D). Taken together, these data showed that particle-based A5 is taken up by immune cells via phagocytic and endocytosis after admixing with CPQ.

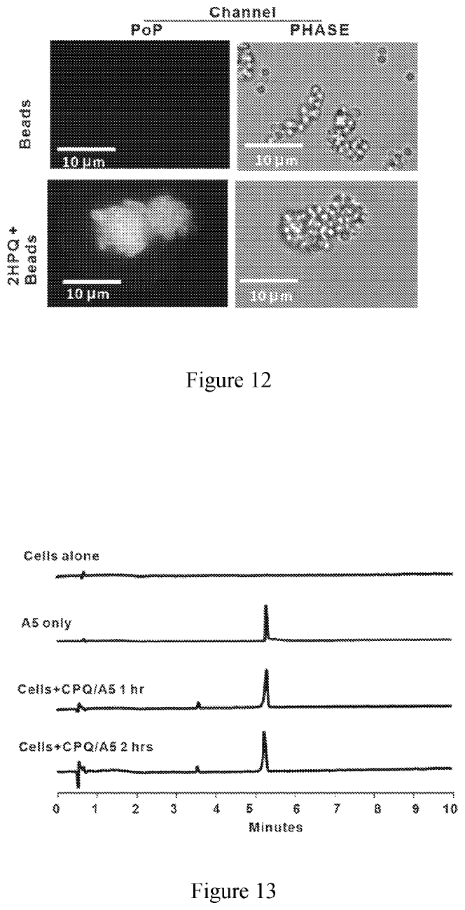

MPLA, which mimics components of the bacterial membrane, has been associated with MHC-I expression within phagosomes. The expression of the H-2L^{d} MHC-I haplotype (which is the restriction element for A5) was assessed following incubation with CPQ/A5 in BMDCs. To assist phagosome visualization, a protocol was developed to coat silica microbeads with CPQ or CPQ/A5 (FIG. 12). Immunofluorescence microscopy was carried out using antibodies (Abs) against the phagosome marker lysosomal-associated membrane protein 1 (LAMP-1), and H-2L^{d}. BMDCs incubated with CPQ beads showed co-localized fluorescence of both H-2L^{d} and LAMP-1, as expected. BMDCs that were incubated with CPQ/A5 showed co-localized fluorescence of all the components; A5, H-2L^{d} and LAMP-1 (FIG. 9E).

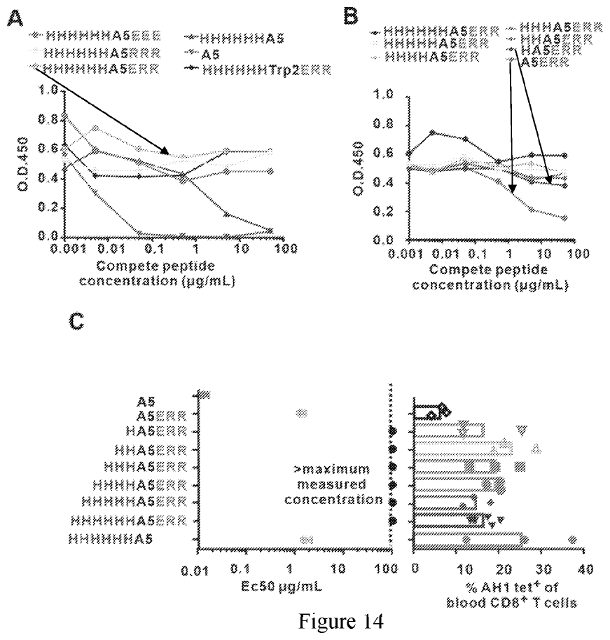

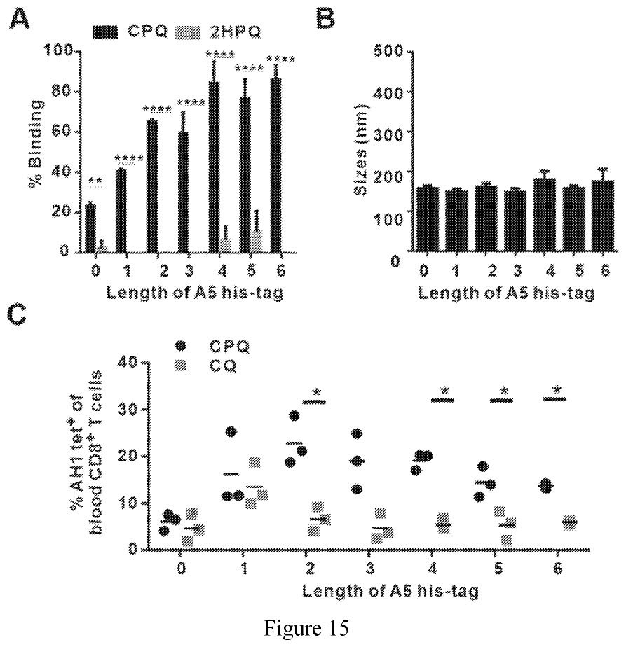

Since the detectable fluorescence signal of A5 within microphages and BMDCs implies it was released from the liposomes and became unquenched, these data suggest that the peptide was released following cellular uptake. Indeed, when incubated with commercial lysosome extract in vitro, A5 release from CoPoP liposomes was detected (FIG. 9F). Almost all the peptide was released from CPQ within 2 hr. The released peptides from macrophage cell extracts could be detected, intact, by high performance liquid chromatography (HPLC). As shown in FIG. 13, the elution time of A5 peptide in the cell lysate is the same as the pure A5 peptide. Taken together, these data suggest that peptides bound to CPQ liposomes are preferentially taken up in APCs, and possibly are released intact to MHC-I directly in cell phagosomes and lysosomes for presentation on their surface. This mechanism would be compatible with the vacuolar pathway for MHC-I peptide presentation, which avoids the necessity for the peptide to be transported first through the cytosol. We note that some studies within this work used the ERR tripeptide on the C terminus to address potential solubility issues, although we later found that this was generally not required in most cases. The presence of this charged tag inhibited peptide binding to recombinant H-2L^{d} in vitro using an enzyme-linked immunosorbent assay (ELISA) assay (FIG. 14). However, the presence or absence of the charged tripeptide did not impact the induction of Ag-specific CD8⁺ T cells. Thus, immunization did not appear overly sensitive to the flanking residues of the MHC-I epitope. Further proteolytic processing of the peptide to remove the small number of adjacent residues to the MHC-I epitope is possible and should be further investigated. It should be noted that shorter abbreviated his-tags on the N terminus resulted in a greater induction of Ag-specific CD8⁺ T cells (FIG. 15). The reason may be related to the enhanced release of the peptide following intracellular uptake as described above.

Antigen Screening Using CPQ

Modern genomic, proteomic, and bioinformatic approaches can rapidly identify extensive lists of coding mutations (i.e., neoantigens) in cancer cells, which have been reported for the murine CT26 and 4T1 cell lines. However, reliably determining which of these are immunogenic and can produce functional responses is not yet realized. Given the potency of the CPQ system, we assessed low-cost peptide micro-libraries to screen candidate peptides. We selected 100 predicted neoantigens on the basis of the strongest MHC-I affinity, as well as several neoantigens that were shared between both CT26 and 4T1 cell lines (Table 3).

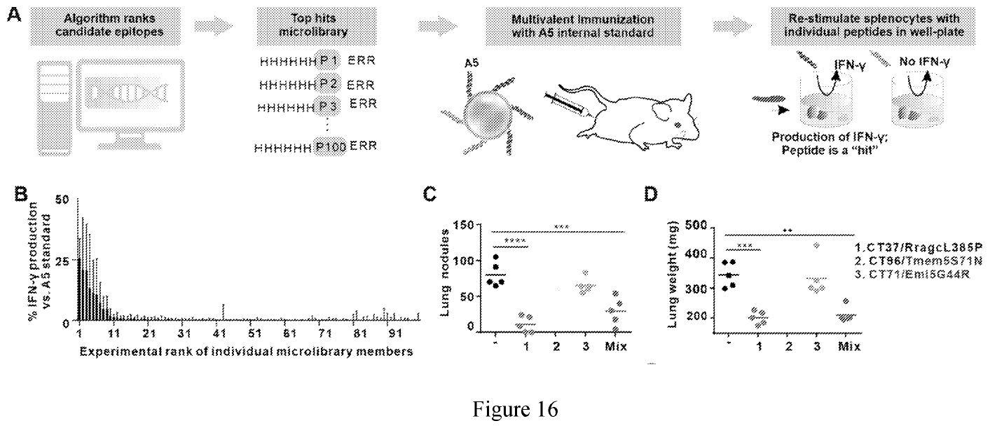

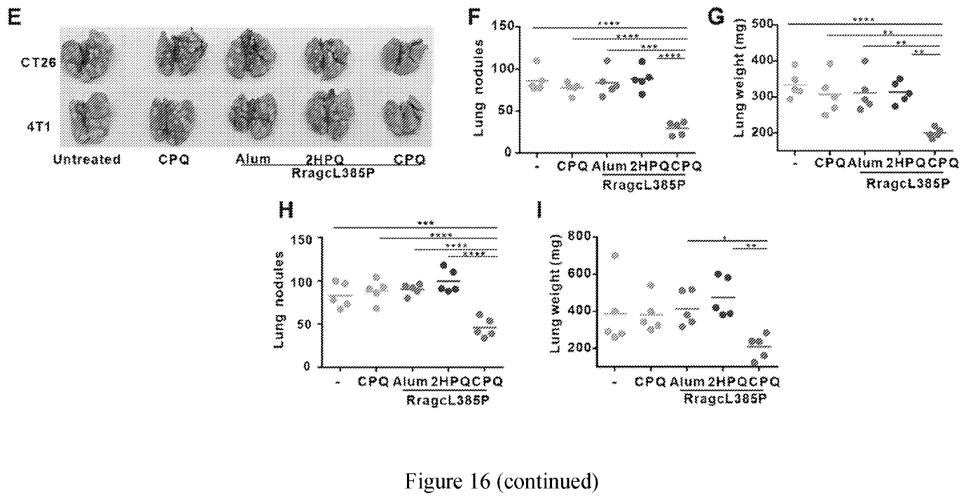

Mice were immunized with 5 library peptides at a time, along with the A5 peptide, with all peptides combined and admixed with CPQ. After two intramuscular injections, splenocytes were collected, and then re-stimulated with each of the synthetic micro-library peptides, individually. The overview of the screening process is shown in FIG. 16A. Production of IFN-γ was measured and peptide immunogenicity was determined relative to the A5 peptide, which served as in internal control that could induce strong Ag-specific CD8⁺ T cell responses.

For Ag screening, multivalent vaccine composed of peptide with the same MHC-I haplotypes (H-2L^{d}, H-2D^{d}, or H-2K^{d}) and the internal A5 peptide were admixed with CPQ. After immunization, only ˜10% of MHC-I binding peptides induced T cells in splenocytes that produced IFN-γ with peptide re-stimulation, and none were nearly as effective as A5 (FIG. 16B). The most highly immunogenic peptides only produced about a quarter of the level of IFN-γ relative to A5. This result underscores the challenges in identifying immunogenic CD8⁺ T cell epitopes from predicted MHC-I binding peptides, even using a strong adjuvant system. One caveat of these results is that we cannot rule out whether the immunodominance of A5 may have had a deleterious impact on the immunogenicity of the other peptides within the cocktail. It is also feasible that immunization with higher antigen doses of single individual peptides could have induced stronger immunogenic responses, although this would decrease the in vivo peptide micro-library screening throughput.

The three most immunogenic 9-mer peptides (RragcL385P, Tmem5S71N and Eml5G44R) were then assessed in the CT26 lung metastasis model. Vaccines were prepared by admixing peptides with CPQ liposomes, followed by two immunizations of 1000 ng (total) of peptides with CPQ, 1 and 8 days after intravenous administration of the tumor cells. Twenty days following challenge, untreated mice had ˜75 lung nodules (FIG. 16C). However, mice vaccinated with CPQ/RragcL385P had just 15 lung nodules. Mice vaccinated with the other two neoantigens showed no significant difference in lung metastasis compared to the untreated mice. Mice vaccinated with a combination of all three of the neoantigens had an average of 25 lung nodules, which was likely attributed to the presence of the RragcL385P Ag. The lung weight confirmed the efficiency of immunization with the RragcL385P vaccine in reducing lung metastases (FIG. 16D).

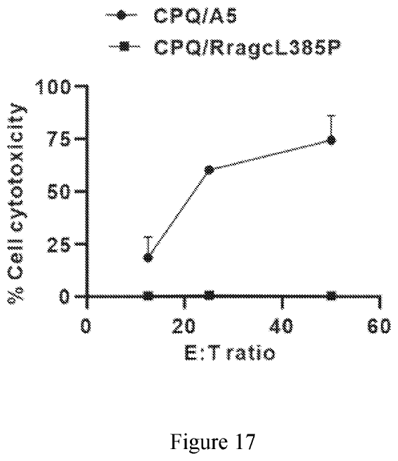

Anti-tumor efficacy of the short RragcL385P peptide (SPKALAHNG (SEQ ID NO:44)) admixed with CPQ was assessed in a therapeutic lung metastasis model. Lungs from mice inoculated with 4T1 tumor cells were collected on day 16 and lungs from mice inoculated with CT26 were collected on day 18. With a 500 ng peptide immunization dose, CPQ/RragcL385P vaccine significantly inhibited lung tumor growth in BALB/c mice (FIG. 16E and FIG. 16F). For mice inoculated with CT26 cells, lungs from control groups (CPQ alone, Alum/RragcL385P, 2HPQ/RragcL385P) had an average of 90 lung nodules and lungs from CPQ/RragcL385P had an average of 30 lung nodules (FIG. 16G). The lung weights reflected the result of the nodule counts (FIG. 16H). For mice inoculated with 4T1 cells, lungs from control groups (CPQ alone, and the peptide mixed with Alum or 2HPQ) had an average of 80 nodules, while lungs from CPQ/RragcL385P had an average of 40 (FIG. 16I); again, the lung weights reflected the results of the nodule counts (FIG. 16J). However, in vitro cell cytotoxicity experiments showed that splenocytes from CPQ/RragcL385P immunized mice were not cytotoxic against CT26 cells, compared to splenocytes from CPQ/A5 immunized mice (FIG. 17), the outcome appeared to be due to an off-target effect that requires further investigation. Thus, although the screening of MHC-I peptides successfully identified a functional epitope, the mechanism of action could not be confirmed to be from tumor cell lysis, so further investigation is warranted to determine the role of immunization with the Rragc epitope. In future work, we still anticipate this screening approach will likely be able to identify peptide epitopes that induce CD8⁺ cytotoxic T lymphocytes (CTLs) capable of epitope-specific tumor cell lysis.

RENCA Neo-Antigen Screening by CoPoP Liposome

We performed whole exome sequencing of mouse kidney carcinoma RENCA cells and tissue cells from healthy BALB/c mice to identify mutations in tumor cells (FIG. 18A). Then NetMHC was used to predict the binding affinity of peptides to MHC-I. Then all of these 20 predicted peptides were admixed with CoPoP liposome to vaccinate healthy BALB/c mice, the immunogenicity of individual peptides was assessed by IFN-γ production after antigen stimulation and the anti-tumor efficacy was assessed by tumor challenge. 8373 mutations were identified in the RENCA cells, 1007 exome coding variants were confirmed by RNA-Seq among these mutations. NetMHC was used to predict the binding affinity of predicated mutated 9 amino acid epitopes. 20 epitopes that has NetMHC percentile less than 0.1 were identified (FIG. 18B and Table 4).

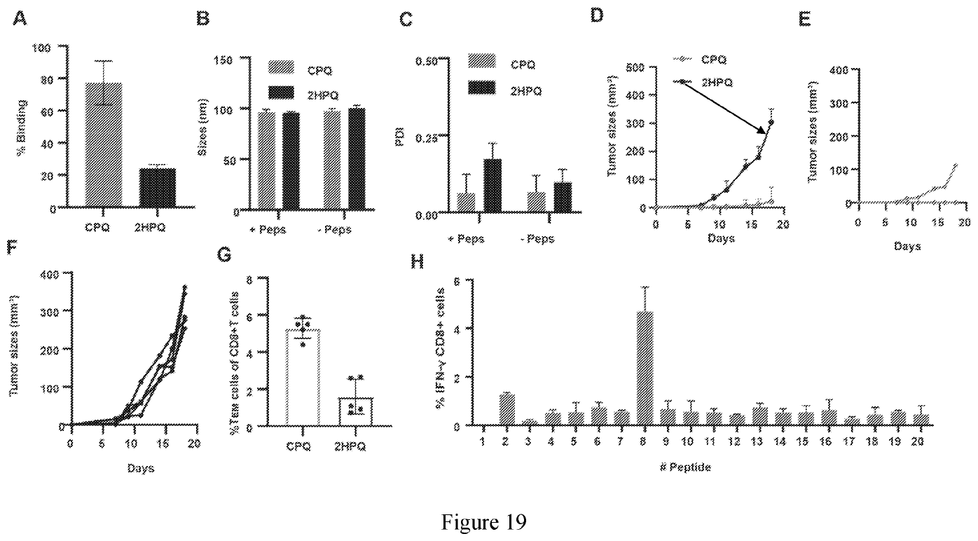

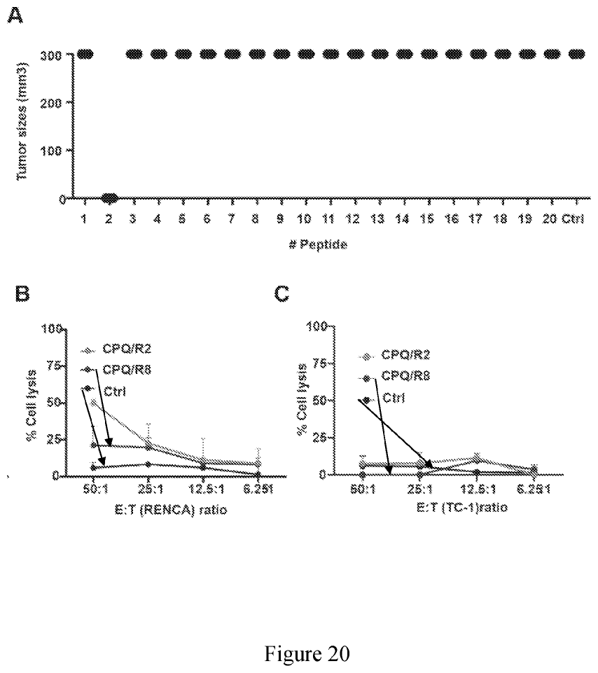

Multivalent vaccine was prepared by first incubate all 20 peptides with CPQ and 2HPQ liposome for a hour at room temperature with a mass ratio of 1:4. The binding percentage of peptides was assessed by peptide filtration assay. There were ˜80% of peptides bind with CoPoP with simple mix, but peptide showed no binding with 2HPQ which is identical liposome as CPQ but lacks cobalt (FIG. 19A). There was no significant change of sizes of liposome before or after binding, which were around 100 nm (FIG. 19B). The polydispersity index of liposomes is smaller than 0.25 (FIG. 19C). Mice were vaccinated with CPQ/peptides on day0 & 7, the injection dose is 50 ng per peptide and lug total peptide per mouse. Mice were then challenged with RENCA cells subcutaneously on day14. Mice vaccinated with CPQ/peptides had significant smaller average tumor sizes compares to 2HPQ/peptides (FIG. 19D). CPQ but not 2HPQ vaccine inhibited tumor growth for 4/5 mice (FIG. 19E) and 2HPQ (FIG. 19F). On day 18, splenocytes were prepared from these mice, CPQ vaccinated mice had significance more effector memory CD8⁺ T cells in the spleen compares to 2HPQ vaccinated mice (FIG. 19G). We also stimulated splenocytes with individual peptide in a 96 wells plate, peptide 2 and peptide 8 had around 2% and 5% of IFN-γ producing CD8⁺ T cells in the spleen but other peptides showed no obvious increasement in IFN-r producing CD8⁺ T cells (FIG. 19H).

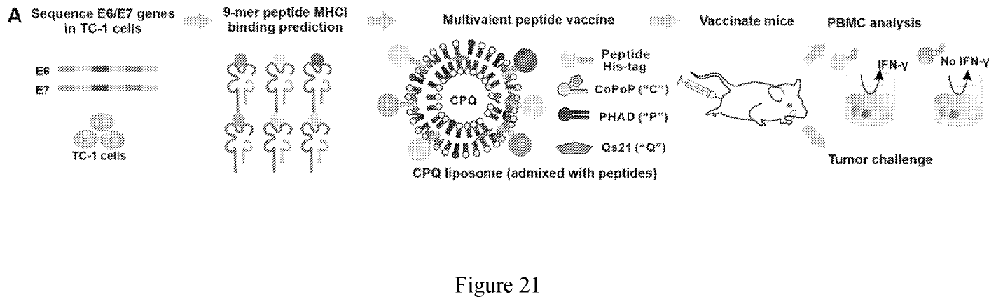

To confirm which peptide was responsible to the anti-tumor efficacy of the vaccine, we admixed single peptide with CoPoP liposome as a vaccine to inject BALB/c mice. These 20 vaccines were injected to 20 groups of BALB/c mice on day 0 & 7, RENCA cells were inoculated on day14. RENCA peptide 2 is the only peptide inhibited tumor growth and all other 19 peptides didn't inhibit tumor growth (FIG. 20A). In vitro cell lysis study confirmed that only peptide 2 induced functional T cells that specific targets to RENCA cells (FIG. 20B) but not TC-1 cells (FIG. 20C).

HPV-16 E6/E7 CD8⁺ T Cell Epitopes Screening by CoPoP Liposome

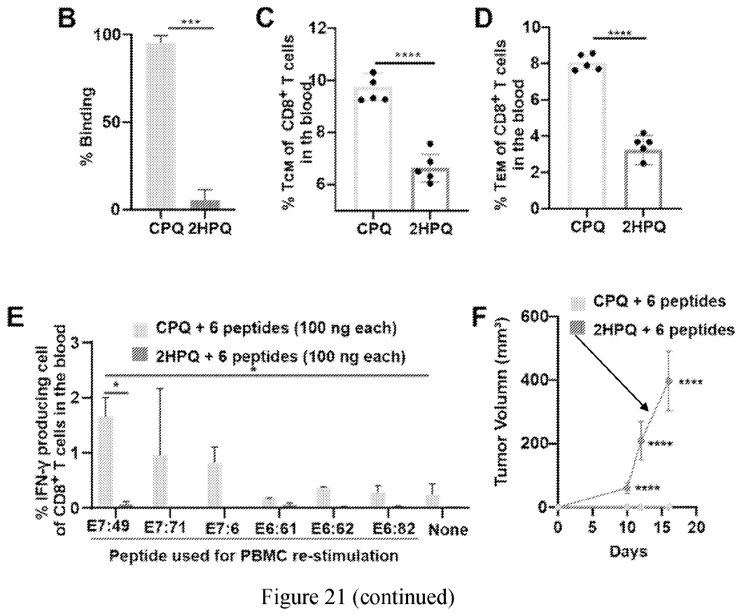

The E6 and E7 genes of TC-1 cells were sequenced from extracted DNA using Sanger sequencing. Six predicted H-2K^{b}- and H-2D^{b}-restricted epitopes within the top percentile of MHC-I binders were identified using a neural network simulation algorithm. These short 9-mer peptides were then chemically synthesized as shown in Table 5, along with a 3-residue polyhistidine sequence to induce particle formation upon admixing with CPQ liposomes. These 6 peptides were combined with CPQ to form a single multivalent vaccine for mice and the immunogenicity of each peptide were assessed by Interferon gamma (IFN-γ production after individual peptide stimulation of collected peripheral blood mononuclear cells (PBMCs). Mice were then challenged with TC-1 cells subcutaneously to assess the tumor protection of the vaccine (FIG. 21A).

The multivalent peptide vaccine was prepared by simple admixing of the 6 peptides with CPQ liposomes at a total peptide to CoPoP mass ratio of 1:4. The dose of each peptide was 100 ng. QS-21 and MPLA were also present in the liposomes at a peptide to each adjuvant mass ratio of 1:1.6. After mixing the 6 pooled E6/E7 epitope candidates with CPQ liposomes for 1 hr at room temperature, ˜100% of the peptides were converted into particle form, as assessed by a microcentrifugal filtration assay (FIG. 21B). Identical 2HIPQ liposomes lacking cobalt, displayed minimal binding with peptides.

To determine immunogenicity, C57BL/6 mice were then vaccinated intramuscularly with the multivalent vaccine on days 0 & 7, and peripheral blood mononuclear cells (PBMCs) were collected and mice challenged with TC-1 tumor cells on day 14. By then, there was a significant increase in the percentage of both central memory T cells (T_{CM}) of CD8⁺ T cells, defined as CD44⁺CD62L⁺ (FIG. 21C), and effector memory T cells (T_{EM}) of CD8⁺ T cells, defined as CD44⁺CD62L⁻ (FIG. 21D) in mice immunized with the peptides mixed with CPQ, but not 2HIPQ. Thus, the presentation of the peptides in the form of a particle, even with identical amounts of QS-21 and MPLA, appeared to be important for inducing memory CD8⁺ T cells.

Collected PBMCs were re-stimulated with the epitope candidates that comprised the multivalent vaccine after which CD8⁺ T cells were assessed for intracellular IFN-γ production, indicating the induction of Ag-specific CD8⁺ T cells. Three peptides induced higher percentage of IFN-γ producing cells of CD8+ T cells over background in the post-immune PBMCs; E7₄₉₋₅₇, E7₇₁₋₇₉ and E7₆₋₁₄. When PBMCs from mice injected with the non-particle forming 2HPQ/peptides were re-stimulated with the peptides, none induced IFN-γ producing CD8⁺ T cells (FIG. 21E). When immunized mice were challenged with TC-1 cells injected subcutaneously, the multivalent CPQ/peptides vaccine group completely rejected tumor growth. In contrast, mice vaccinated with the 2HPQ/peptides formulation developed rapidly growing tumors (FIG. 21F).

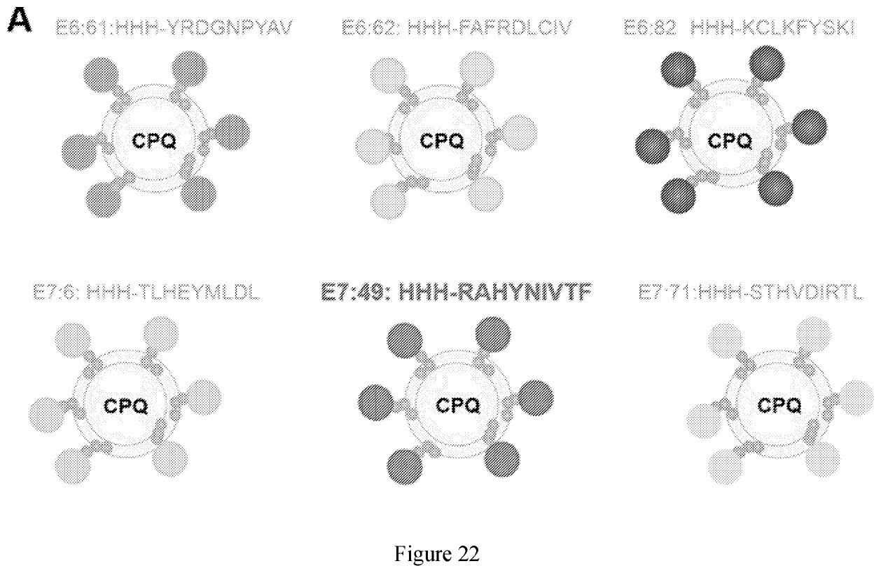

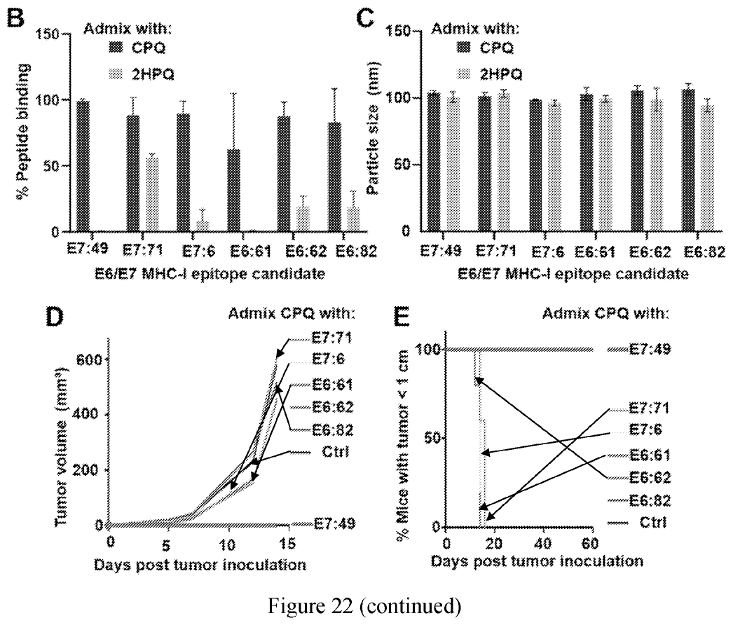

Next, to determine which of the peptide epitopes provided anti-tumor activity, peptides were admixed individually with CPQ or 2HIPQ liposomes (FIG. 22A). Individually, all of the peptides effectively bound to CPQ liposomes, but not to 2HIPQ liposomes lacking cobalt (FIG. 22B). Liposome size remained ˜100 nm after binding with E6 and E7 peptides (FIG. 22C). Next, mice were vaccinated with the peptides admixed with CPQ on days 0 & 7 at a peptide dose of 500 ng. Each mouse was then challenged with TC-1 cells on day 14. Of all the synthetic short peptide epitopes assessed, only the E7_{HHH49-57} peptide (sequence: HHH-RAHYNIVTF (SEQ ID NO:8)) inhibited tumor growth (FIG. 22D). Prolonged tumor-free survival was maintained to at least 60 days (FIG. 22E) reflecting the observational period, while no other predicted peptide epitopes had any measurable anti-tumor efficacy.

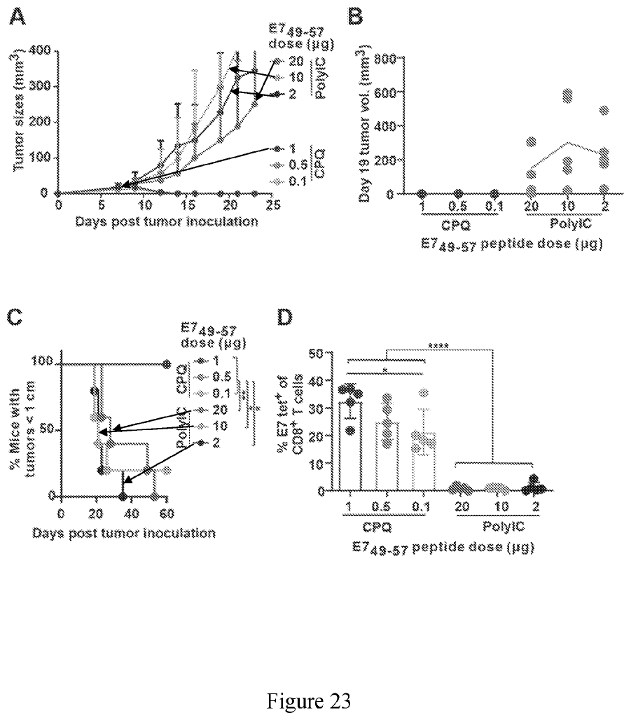

Since CPQ/E7_{HHH49-57} was effective in a prophylactic setting, we next assessed the vaccine on established TC-1 tumors, which is also the setting in which most cancer vaccines would be initially tested clinically. Poly(I:C) was used as an adjuvant comparator. For poly(I:C), the synthetic short peptide dose ranged from 20 pg to 2 pg per mouse while the poly(I:C) dose remained fixed at 20 pg/mouse. For the CPQ liposome adjuvant, the peptide dosing varied from 1 to 0.1 pg per mouse. Given that the ratio of liposomes to peptide was fixed, this corresponds to a 3D6A-PHAD dose and QS-21 dose ranging from 1.6 to 0.16 μg, along with a CoPoP dose ranging from 4 to 0.4 μg. The identical E7_{HHH49-57} peptide was admixed with either of the adjuvants just prior to intramuscularly immunization. Mice were first inoculated with TC-1 cells on day 0 and then vaccinated on days 2 and 9. Under these conditions, only the CPQ adjuvant, but not the poly(I:C) adjuvant resulted in effective tumor growth inhibition (FIG. 23A). On day 19, CPQ vaccinated mice had no detectable tumor growth, but nearly all the of the poly(I:C) vaccinated mice did, even at the highest dose of peptide (20 μg) (FIG. 23B). The tumors of all the poly(I:C)-vaccinated mice reached the 1 cm endpoint within 60 days, whereas all CPQ-vaccinated mice remained tumor-free even at just a 0.1 μg E7 peptide dose, which reflects a 200-fold lower dose compared to the upper peptide dose with poly(I:C) (FIG. 23C). As anti-tumor efficacy would be expected to relate to the frequency of Ag-specific CD8⁺ T cells, we examined these 19 days post-tumor inoculation. In the CPQ vaccinated mice, there were higher percent of CD8⁺ T cells that were Ag-specific in the blood, with increasing peptide dose. A peptide dose of 1 μg resulted in ˜30% tetramer-positive (tet⁺) CD8⁺ T cells, whereas, a peptide dose of 0.1 μg resulted in tet⁺ conversion of ˜20% CD8⁺ T cells. In strong contrast, poly(I:C) vaccinated mice had almost no Ag-specific CD8⁺ T cells in the blood (FIG. 23D).

CoPoP as enhanced mimotope screening system for Trp2 peptide we developed an improved mimotope evolution system, as outlined in FIG. 24A. The Tyrosinase-related protein 2 epitope (Trp2₁₈₀₋₁₈₈; SVYDFFVWL (SEQ ID NO:45)), a mouse H-2K^{b} and human HLA-A2 epitope was used as a model Ag. To develop a e-mimotope, we first obtained nine positional random libraries by inserting all 20 amino acid on the certain position and fix 8 positions of a peptide with the original amino acid of the wild type peptide. Thus, the method in one embodiment comprises an iterative amino acid randomization of candidate peptides. For each positional library, all 20 amino acid were present in at specific residue locations. C57BL/6 mice were then immunized on day 0 & 7 with the libraries (1 μg total peptide library dose). On day 14, mice were challenged with B16-F10 cells, which express Trp2, and tumor growth was monitored. Remarkably, a week after tumor inoculation, mice vaccinated with the Trp2 position 8 peptide library had significant smaller tumor sizes compared to all micro-libraries (FIG. 24B). Next, 20 individual Trp2 peptide variants were synthesized, with each peptide bearing a different amino acid at position 8. Mice were immunized with these peptides with CPQ individually. As shown in FIG. 24C, following tumor challenge, mice immunized with Trp2-8C showed inhibition of tumor growth on day 21, compared to the wild-type sequence (Trp-8W). Immunization with Trp-8Y also completely inhibited tumor growth in one mouse. Trp2-8C and Trp2-8Y were further studied to confirm whether the mimotope could inhibit tumor growth relative to the wild type epitope (Trp-8W).

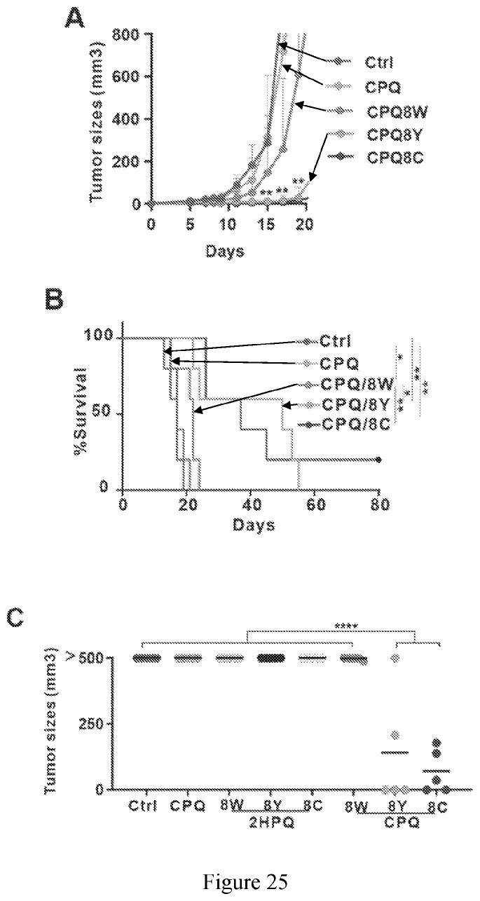

Mice were immunized with CPQ and 500 ng of peptide on day 0 & 7; then B16-F10 cells were inoculated on day 14. Mice immunized with Trp2-8Y and Trp2-8C had significantly delayed tumor growth compared to mice immunized with the wild-type Trp2-8W, or the adjuvant alone, with tumor growth in those groups similar to untreated mice (FIG. 25A). CPQ/Trp2-8C and CPQ/Trp2-8Y prolonged mice survival compares to CPQ/Trp2-8W (FIG. 25B). By day 23, only mice vaccinated with Trp2-8Y and Trp2-8C had tumor sizes smaller than 500 mm³ (FIG. 25C).

Positional Peptide Libraries as Immunogens for CPQ Liposomes

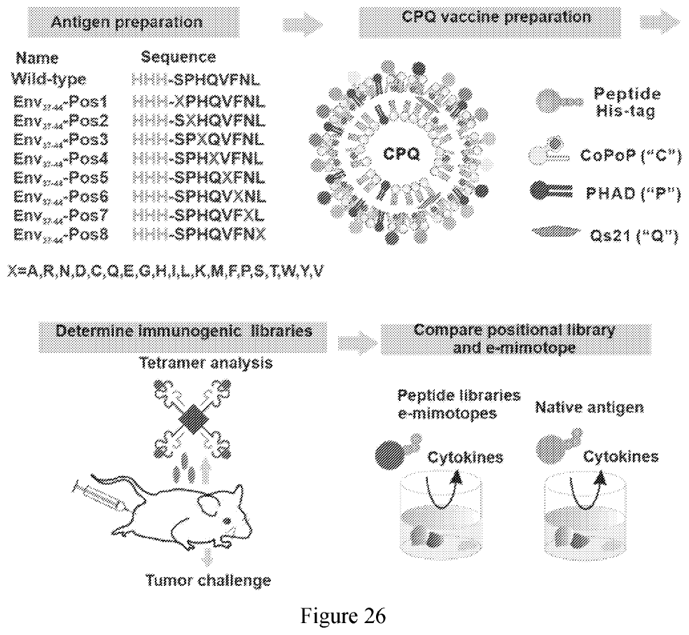

Position-scanning libraries were synthesized as shown in FIG. 26. Three histidine were added to the N-terminus of all peptides and peptide libraries for particle formation with CoPoP liposomes. For each library, the amino acid at a specific position was substituted with all 20 amino acids while the remainder of the positions were kept the same as the wild-type sequence. For this 8-mer peptide, we made 8 positional libraries. Peptides were combined with CPQ to form a single peptide vaccine or positional peptide library vaccine for mice. The immunogenicity of these immunogens was assessed by the wild-type antigen Env₃₇₋₄₄ tetramer staining; and the anti-tumor efficacy of these immunogens were assessed by challenging mice with CT26 cell subcutaneously. The cross-reactivity of T cells that induced by positional peptide libraries and single peptide mimotopes to native peptides were assessed by cytokine production after in vitro antigen stimulation of splenocytes.

The Immunogenicity of Env₃₇₋₄₄ Positional Peptide Libraries

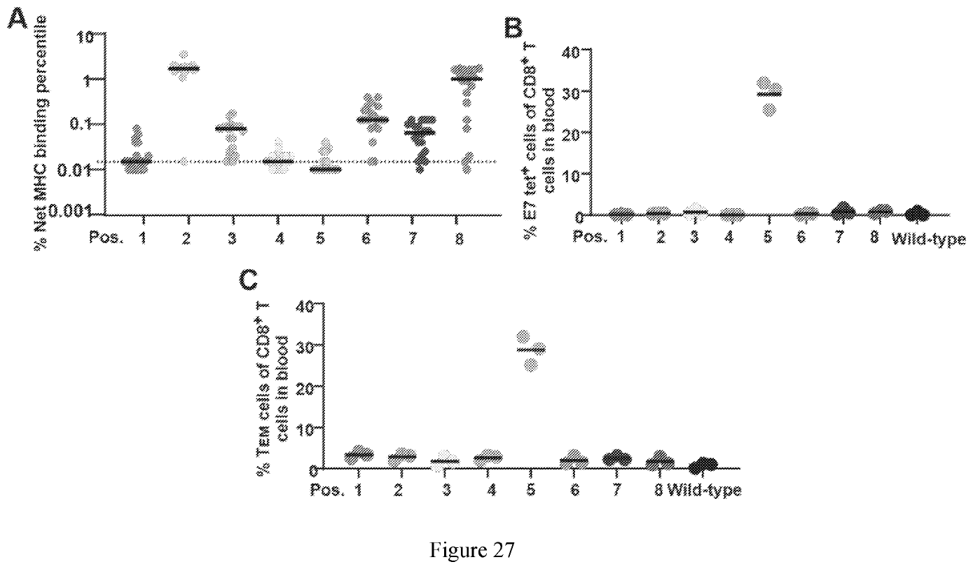

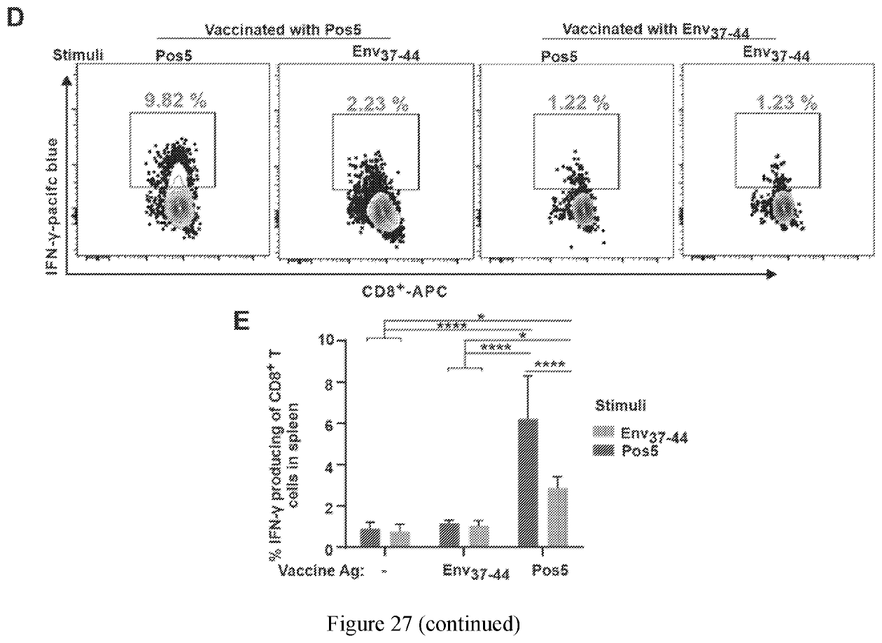

The NetMHC neural network algorithm was used to predict the binding affinity of each positional library. The H-2L^{d}-binding percentile of wild-type Env₃₇₋₄₄ is 0.015%, which represents an extremely good MHC-I binder. The individual library members of Env₃₇₋₄₄-Pos5 were predicted to have, in general, only slightly poorer binding compared to native epitope (FIG. 27A). Since Env₃₇₋₄₄ is a H-2L^{d}-reactive peptide, and the amino acid Pro at the second residue is a binding motif for H-2L^{d}, Pos2 library members that had substitutions at position 2 had orders of magnitude poorer H-2L^{d} binding. We next used the 8 positional peptide libraries to immunize compare immunogenicity to the native Env₃₇₋₄₄ vaccine. In all cases, the peptides were admixed with CPQ liposomes to induce particleized peptides. An Env₃₇₋₄₄ tetramer was used to assess Env₃₇₋₄₄ specific CD8⁺ T cells. BALB/c mice were immunized on days 0 and 7; then blood was collected on day 14 for tetramer analysis. Of all the positional libraries assessed, only CPQ/Env₃₇₋₄₄-Pos5 vaccine induced 30% of CD8⁺ T cells that were Env₃₇₋₄₄ specific, while other peptide library vaccines or the wild-type epitope vaccine did not elicit any detectable Env₃₇₋₄₄ specific CD8⁺ T cells (FIG. 27B). CPQ/Env₃₇₋₄₄-Pos5 vaccine induced ˜30% of effector-memory T cells within the CD8⁺ T cell population (FIG. 27C; CD62L⁻CD44⁺); in contrast, mice vaccinated other positional libraries or the wild-type peptide induced no detectable effector-memory T cells. To understand this remarkable efficacy of the mimotope library, we collected splenocytes from untreated mice or mice vaccinated with CPQ/Env₃₇₋₄₄-Pos5 or CPQ/Env₃₇₋₄₄ 23 days after tumor inoculation. Splenocytes from Pos5 vaccinated mice had ˜6% of CD8⁺ T cells that produced IFN-γ after Pos5 stimulation and ˜2-3% of CD8⁺ T cells produced IFN-γ after wild-type peptide stimulation (FIG. 27D and FIG. 27E). However, splenocytes from untreated mice or mice vaccinated with wild-type peptide showed no detectable IFN-γ production neither after Env₃₇₋₄₄-Pos5 stimulation nor wild-type peptide stimulation. It is important to note that these CD8⁺ T cells elicited by the Env₃₇₋₄₄-Pos5 vaccine produced more IFN-γ in response to Env₃₇₋₄₄-Pos5 stimulation compared to wild-type peptide stimulation. This indicates that CD8⁺ T cells elicited by Env₃₇₋₄₄-Pos5 vaccine bound with Env₃₇₋₄₄ tetramer and had higher affinity with Env₃₇₋₄₄-Pos5 compares to with Env₃₇₋₄₄.

AH1 Positional Peptide Libraries as Immunogens for CPQ Liposomal Vaccine

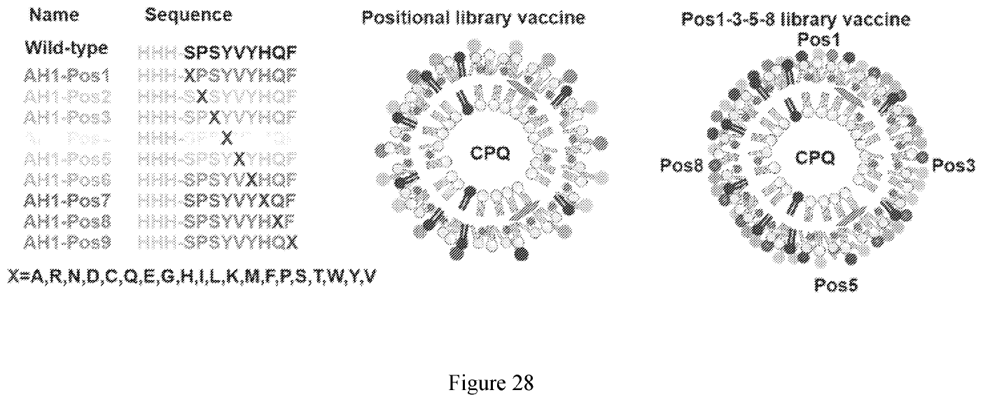

Next, we applied the positional library vaccine strategy to second MuLV epitope; AH1. Unlike Env₃₇₋₄₄, AH1 is an established tumor rejection epitope expressed highly on MHC-I of CT-26 cancer cells.^{[15]} Positional libraries were generated for each amino acid position of the 9mer AH1 sequence as shown in FIG. 28.

Anti-tumor efficacy of AH1 positional peptide libraries

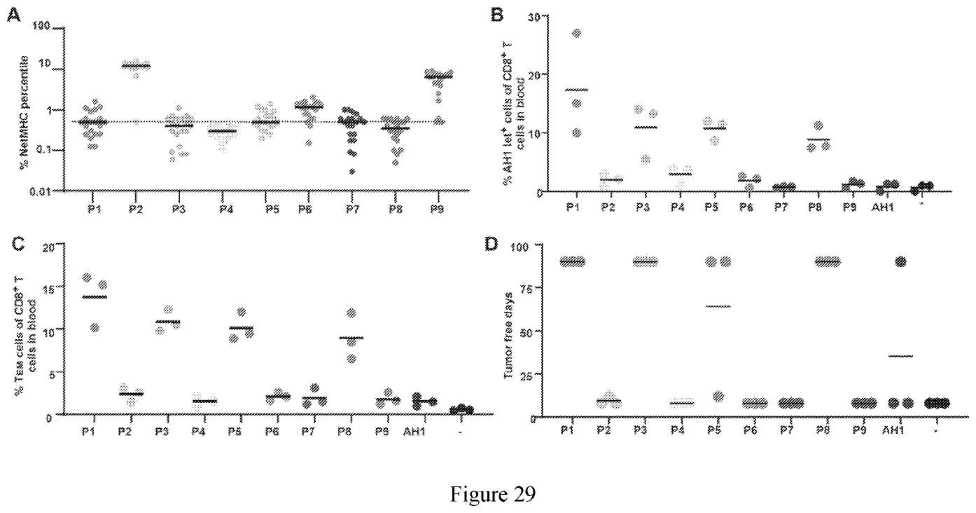

We first predicted the MHC-I binding affinity of AH1 libraries by NetMHC,^{[20]} The binding percentile of wild-type AH1 to the H-2L^{d} is 0.5%, which indicated that AH1 is a good MHC-I binder, and any amino acid change on positions 2 and 9 diminishes the MHC-I-peptide binding, but amino acid changes in other positions were not predicted to alter the average H-2L^{d} binding significantly (FIG. 29A). BALB/c mice were then immunized with CPQ and AH1 libraries or the wild-type AH1 peptide at 1 μg total peptide dose on day 0 and day 7 and blood was collected on day 14 for analysis. The wild-type AH1 peptide did not elicit detectable amount of AH1 tet⁺ CD8⁺ T cells but mice vaccinated with CPQ and peptide library Pos1, Pos3, Pos5 or Pos8 elicited more than 10% of CD8⁺ T cells that were Ag specific (FIG. 29B). These Ag specific T cells were effector memory T cell phenotype (FIG. 29C). Vaccine CPQ/P1, CPQ/P3. CPQ/Pos8 protected 100% mice from tumor challenging for at least 90 days. CPQ/Pos5 protected 2/3 mice from tumor challenging for at least 90 days (FIG. 29D). Only a single mouse was protected with the wild-type CPQ/AH1 immunization.

Identification of AH1 e-mimotope that has anti-tumor efficacy upon vaccination

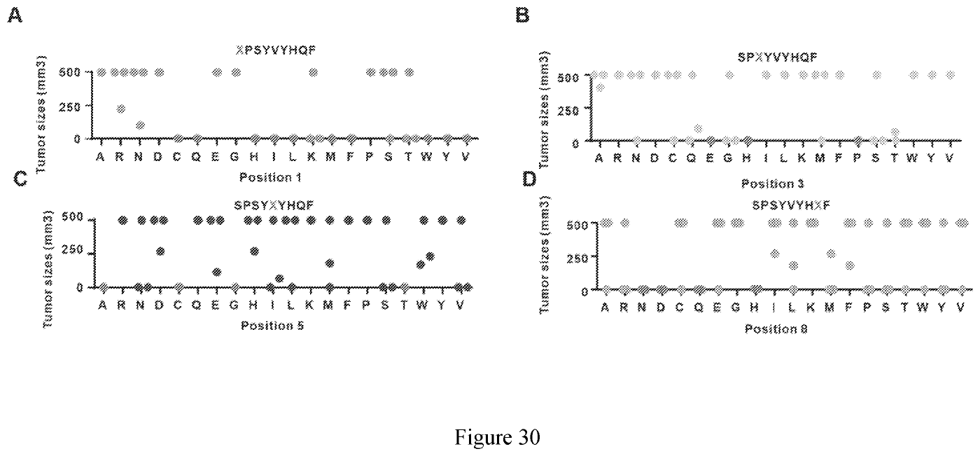

Next, 20 individual AH1 peptide variants were synthesized, with each peptide bearing a different amino acid at pos1, 3, 5, 8. Mice were immunized with these CPQ/peptide vaccines individually, to keep constant with the first position library screening, the injection dose of single peptide is 50 ng. As shown in FIG. 30A, for pos1, around 10 peptide variants with amino acid replacement (C, Q, H, I, L, M, F, W, Y, V) inhibited CT26 tumor growth. For pos3, 3 peptide variants with amino acid replacement (E, H, P) 100% inhibited CT26 tumor growth (FIG. 30B). For pos5, 4 peptide variants with amino acid replacement (A, C, G, T) 100% inhibited CT26 tumor growth (FIG. 30C). For position8, 4 peptide variants with amino acid replacement (N, D, Q, H) 100% inhibited CT26 tumor growth (FIG. 30D).

Conclusions

CPQ liposomes induced stable particle formation of short peptides and were demonstrated to be highly effective for inducing Ag-specific CD8⁺ T cells that inhibited tumor growth in several multiple mouse tumor models in both local and metastatic settings. Immunization was well-tolerated in mice. The putative mechanism of potency is related to encouraging infiltration of APCs into draining lymph nodes, enhanced delivery of the short peptide to APCs, followed by the putative release of the peptide for binding to MIC-I expressed within endosomes and phagosomes. Based on this potency, micro-libraries were screened to identify a shared epitope, RragcL385P, that reduced metastatic disease when vaccinated together with CPQ in both CT26 and 4T1 cell lines, although this epitope appeared to operate from an off-target effect, which requires further study to understand the basis for this observation. The same method was used to screen neo-antigen for RENCA cell line, peptide (AYTTQREEL (SEQ ID NO:43)) were discovered as a neo-antigen for RENCA tumor cell line, it delayed tumor growth when admix with CPQ liposome as a vaccine. We also use CPQ liposome as a screening tool to screen the HPV-16 cellular oncoproteins E6 and E7. Of the peptides screened, only the previously identified E7₄₉₋₅₇ epitope was functional. Immunization with the synthetic short peptide at a dose of 100 ng protected mice in a therapeutic tumor challenge when admixed with CoPoP liposomes, whereas 200-fold higher peptide doses were ineffective with the Poly(I:C) adjuvant. We also developed a method to use CPQ to find Trp2 and AH1 e-mimotope with improved function compared to the native epitope. Furthermore, we found that positional library itself can serve as a vaccine immunogen. The CPQ system can be used with other MIC-I epitopes, as well as for embodiments dedicated to functional epitope screening and discovery.

Experimental

Materials

Co(II)PoP was synthesized by methods known in the art. The following other lipids were used: DOPC (Corden; catalog number: LP-R4-070), Ni-NTA lipid dioleoylglycero-Ni-NTA (Avanti; catalog number: 790404P), cholesterol (PhytoChol; Wilshire Technologies), synthetic PHAD (Avanti; catalog number: 699800P). QS-21 was obtained from Desert King (catalog number: NC0949192). The following adjuvants were obtained: Alhydrogel 2% aluminium gel (Accurate Chemical and Scientific Corporation; catalog number: A1090BS). Poly (I:C) (Sigma; catalog number: P1530). Granulocyte-macrophage colony-stimulating factor (GM-CSF) was obtained from Shenandoah Biotechnology (catalog number: 200-15-AF). Chlorpromazine hydrochloride was obtained from VWR (catalog number: TCC2481). Cytochalasin B was obtained from Acros (catalog number: 228090010). Lysosomes was obtained from Xeno tech (catalog number: H0610.L). 10× catabolic buffer was obtained from Xeno tech (catalog number: K5200). The following antibodies were obtained from BioLegend, APC-CD8a antibody (Catalog number:100712), FITC-I-A/I-E antibody (catalog number:107605), FITC-B220 (Catalog number:103206), FITC-CD4 antibody (catalog number:100405), PerCP/Cyanine5.5-CD44 (Catalog number:103031), PE/Cy7-CD62L antibody (catalog number:104417), pacific blue IFN-γ (catalog number: 505818), PE-TNFα (catalog number: 506305), Alexa Fluor 488-Ly6C (catalog number: 128021), PE/Cy7-CD11b (catalog number: 101215), PE-Ly6G (catalog number: 127607), APC/Cy7-CD11c (catalog number: 117323), PerCP/Cyanine5.5-CD3 (catalog number: 100217), Alex Fluor 700-I-A/I-E (catalog number: 107621), Brefeldin A (BD, Catalog number: 555029), live/dead fixable dye (Invitrogen; catalog number: L34965), fixation/permeabilization kit (BD; catalog number: 554714). Cell lysis buffer was obtained from BioVision (Catalog number: 5830). A5-HiLyte488 and E7-HiLyte488 were synthesized by Anaspec. Collagenase Type I (Gibco; catalog number: 17018-029), DNase I (Roche Diagnostics; catalog number: 04536282001)

Vaccine Preparation and Characterization

Liposomes were prepared by ethanol injection and lipid extrusion as reported previously. The prepared liposomes were dialyzed in phosphate buffered saline (PBS) at 4° C. to remove ethanol and passed through a 0.2 m sterile filter. For liposomes containing QS-21, QS-21 (1 mg/mL) was added to liposomes overnight at 4° C. with the [DOPC:Chol:CoPoP/PoP:PHAD:QS-21] mass ratio of [20:5:1:1:1]. The final liposome concentration was adjusted to 320 μg/mL CoPoP; we did not actually measure individual lipid concentrations, but operated on the assumption that the input concentration was maintained.

To prepare CPQ, CP, CQ, 2HP and 2HPQ vaccine, liposome and peptides were incubated at mass ratio of 4:1 for 1 hr at room temperature. To prepare PQ+C/A5 vaccine, A5 peptide was incubated with CoPoP liposomes (lacking PHAD or QS-21) for an hour, then 2HPQ liposome was added to the sample immediately before injection. For desired Ag dosing, liposomes were incubated with Ag, as described above, then diluted in PBS. To prepare Alhydrogel (Alum) vaccines, A5 was mixed with 2% Alum for an hour and diluted with 4-(2-hydroxyethyl)-1-piperazineethanesulfonic acid (HEPES) buffer before injection. Each vaccine contained 500 ng of peptide and 75 μg Alum. To prepare the poly(I:C) vaccine, the peptide was mixed with poly(I:C) for 1 hr and then further diluted in PBS for a dose of 500 ng peptide and 50 μg poly(I:C) for A5 study and 20 μg poly(I:C) for TC-1 study.

To characterize binding of liposomes and peptides, peptides were incubated with liposomes or PBS for 1 hr at room temperature and subjected to micro-centrifugal filtration tube with a 100 kDa cutoff (PALL; catalog number: 29300) to separate free peptide from liposomes. Micro BCA (Thermo; catalog number: 23235) assays were used to determine the amount of free peptide in the filtrate. Percentage binding of peptides to liposomes was calculated by comparing liposome-bound peptides to free peptide. Dynamic light scattering with a NanoBrook 90 plus PALS instrument was used to measure sizes and polydispersity indexes of 500-fold diluted sample in PBS.

To characterize the serum stability of vaccine, pre-prepared A5-HiLyte488 or E7-HiLyte488 and liposome mixtures were incubated in 40% human serum or 10% FBS in PBS at 37° C. Samples were diluted in PBS for fluorescence measurement at different time points. The fluorescence of peptide was quenched once peptide bind to liposome due to energy transfer from HiLyte488 to porphyrin. The percentage binding of peptide was calculated based on the percent of fluorescence quench of peptide.

For the in vitro release of peptide in lysosome study, lysosome solutions were prepared as described in the manufacture's instruction. Briefly, in a 96 wells plate, 10 μL 10× catabolic buffer was mixed with 50 ul 1× lysosome and 40 ul water. Prepared CPQ/A5-HiLyte488, CoNTA/A5-HiLyte488 or PBS/A5-HiLyte488 were added to lysosome solution and incubated at 37° C. The fluorescence of the mixture was measured at indicated time points. The fluorescence of A5-HiLyte488 was read with 491 nm excitation and 527 nm emission in a microplate reader (TECAN Safire).

Cryo-Electron Microscopy

To analyze the morphology of CPQ liposomes before and after binding of A5 peptide, approximately 3.6 μL of each sample was applied to the holey carbon grids and manually blotted using the Vitrobot blotting paper (Standard Vitrobot Filter Paper, 055/20 mm, Grade 595). Right after blotting, a new drop of the sample was applied to the EM grid and blotted again using the standard routine with the two blotting pads in the Vitrobot Mark IV (Thermo Fisher Scientific) for 3 sec and a blot force +1. The grid was then immediately plunged into liquid ethane. The Vitrobot was set at 25° C. and 100% relative humidity. For all samples, we used c-flat grids (C-Flat 2/2-3Cu-T), which were washed with chloroform for 2 hr negative glow discharge in air at 5 mA for 15 seconds right before the sample was applied for vitrification. Samples were imaged in a Tecnai F20 electron microscope operated at 200 kV using a side-entry Gatan 626 single tilt cryo-holder. Images were collected in a TVIPS XF416 CMOS camera at a magnification of 50,000×, which produced images with a calibrated pixel size of 2.145 Å. Images were collected with a total dose of ˜10 e-/λ2 using a defocus ranging from −1.75 to −2.50 μm.

Sequencing of RNA from Healthy BALB/c Mice and RENCA Tumor Cells

Whole exome sequencing library was prepared using the “SureSelect^{XT} Mouse All Exon Kit” from Agilent according to the manufacturer's instructions. Pair-end sequencing was performed on Illumina NextSeq platform to produce 75 bp reads. For the RNA-Seq experiment, we used the Illumina Stranded TruSeq RNA library preparation kit, followed by 75-cycle paired-end sequencing on the NextSeq in mid-output mode, generating approximately 25 million reads per sample.

To make variant calls from whole exome sequencing data, we first aligned the raw sequencing reads (in fastq format) to the GRCm38 reference assembly using BWA (version 0.7.13, with the “mem -M” option), followed by merging and sorting (by genomic coordinates) of the individual fastq files from the same sample sequenced on multiple flow-cell lanes. We then used the Mutect2 tool from the Genome Analysis Toolkit (GATK, version 4.0.9.0) with default parameters to make variant calls using the coordinate-sorted bam file from tumor cell line as input for ‘-tumor’ and the bam file from BALB/c as input for ‘-normal’. We then filtered the variants in the resulting VCF file using the “FilterMutectCalls” tool in GATK. The filtered VCF files were further normalized by splitting multiple alleles and left-aligning indels using the bcftool in the samtools suite (samtools.github.io/bcftools/bcftools). The final VCF file was used as input to the online Ensembl Variant Effect Predictor (useast.ensembl.org/Mus_musculus/Tools/VEP) tool for variant functional effect annotation. For RNA-seq data, we first aligned the raw sequencing reads from the tumor cell lines as well from the Balb/c sample to the GRCm38 reference genome using STAR (version 2.6.1b_10-01) with the two-pass approach. The resulting bam files are used to make variant calls using Mutect2. We then filtered the vcf files using the ‘VariantFiltration’ tool in GATK with “-window 35-cluster 3-filterName FS filter “FS>30.0”-filterName QD-filter “QD<2.0” as parameters. To get the final candidate nonsynonymous variants, we filtered the variants in the exome sequencing VCF file that are annotated as nonsynonymous and are also detected in RNA-Seq data with minimum read depth of four. We then extracted the peptide sequence around these nonsynonymous variants using a custom Perl script and used the resultant peptide sequence as input for binding affinity prediction using the NetMHC-I binding prediction server (cbs.dtu.dk/services/NetMHC/).

Cell Studies

RAW264.7 murine macrophage cells were obtained from the American Type Culture Collection (ATCC) and cultured in Dulbecco's modified Eagle's medium (DMEM) with 10% fetal bovine serum (FBS) and 1% penicillin/streptomycin. CT26 colon cells were obtained from ATCC and cultured in RPMI 1640 with 10% FBS and 1% penicillin/streptomycin (pen/strep). The 4T07 cell line was kindly provided by Dr. Josh Gamble (Karmanos Cancer Institute, Detroit, MI) and cultured in DMEM containing 10% FBS and 1× Glutamine and 1% pen/strep. CMS4-met cells were kindly provided by Dr. Abrams (Roswell Park, Buffalo, NY) and cultured in Roswell Park Memorial Institute (RPMI) 1640 media containing 10% FBS and 1% pen/strep. 4T1 cells were kindly provided by Dr. Yun Wu (University at Buffalo, Buffalo, NY) and cultured in RPMI 1640 with 10% FBS and 1% pen/strep. RENCA cells were obtained from American Type Culture Collection (ATCC) and cultured in RPMI1640 supplemented with 10% Fetal Bovine Serum, 0.1 mM extra Non-essential amino acids (NEAA), 1 mM extra sodium pyruvate, 2 mM extra L-glutamine. TC-1 cells were obtained from Dr. Gomez-Gutierrez, (university of Louisville, Kentucky) and cultured in Dulbecco's modified Eagle's medium (DMEM) with 10% FBS and 1% penicillin/streptomycin (pen/strep). B16-F10 cells were obtained from ATCC and cultured in DMEM with 10% FBS and 1% penicillin/streptomycin. BMDCs were derived from bone marrow from the femurs and tibia of BALB/c mice. 10⁷ cells/mL were cultured in 10 mL RPMI 1640 culture medium with 10% FBS, 1% pen/strep, and 20 ng/mL of recombinant murine GM-CSF. On day 3, an additional 10 mL media containing GM-CSF was added, so the final volume was 20 mL. On day 6, non-adherent cells were collected and cultured in a 24-well plate at 5×10⁵ cell/mL in RPMI 1640 culture medium containing 10% FBS and 1% pen/strep. For the splenocyte studies, freshly isolated spleens were dissociated and filtered through a 70 μm cell strainer. The plunger from a sterile 3 mL syringe was used to dissociate tissue through the strainer, 5 mL of cold PBS was used to wash cells into a 50 mL tube. Cells were centrifuged at 500×g for 5 min, the supernatants were discarded. Red blood cells were lysed with a 5 mL red blood cell lysis buffer for 5 min, then 35 mL PBS was added to the tube. Cells were centrifuged again and the cell pellets were collected for further use. Splenocytes were cultured in RPMI 1640 supplemented with 10% FBS, 1% pen/strep, 2 mM glutamine, 1 mM sodium pyruvate, 1× non-essential amino acids solution and 50 pM 0-Mercapethanol. Cells were cultured in 5% CO₂/95% air at 37° C. in a humidified chamber.

For in vitro cell uptake studies, RAW264.7 cells (2.5×10⁵ per well) and BMDCs (2.5×10⁵ per well) were cultured in a 24-well plates overnight, then treated with CPQ/A5-HiLyte488, 2HPQ/A5-HiLyte488 and PBS/A5-HiLyte488 (peptide concentration of 1 μg/mL) for 10 min, 30 min or an hour. For phagocytosis and endocytosis inhibitor study, cells were first pre-incubated with cytochalasin B (10 μg/ml) or chlorpromazine (10 μg/mL) for an hour before the cell uptake study. Cells were washed and lysed with 0.1% Triton X-100 and 10 mM dithiothreitol (DTT). The fluorescence signals were measured before and after adding DTT. Cellular A5-HiLyte488 uptakes were calculated by preparing an A5-HiLyte488 standard curve.

For HPLC of cell lysate: 1×10⁶ RAW 264.7 murine macrophages were seeded in a T25 cell culture flask until confluent. CPQ/A5-HiLyte488 (peptide concentration of 2 μg/mL) or PBS was added to cell culture medium for the indicated hours. Cells were washed, lysed and centrifuged. Supernatant was collected and injected to a reversed phase HPLC column Agilent poroshell 120 EC-C18 (2.7 μm packing, 4.6×50 mm length). The mobile phase consisted of acetonitrile and 0.1% Trifluoroacetic acid (TFA) in water and the method was 5% to 60% acetonitrile for 10 mins at 1 mL/min. The HPLC system consist of Agilent Technologies 1260 Infinity and a Diode-array detector (G1315C DAD VL+) set at 475 nm.

Murine Studies

In vivo immunization: Murine studies were performed according to protocols approved by the University at Buffalo IACUC. 5-6 week-old female BALB/c mice (Charles River Laboratories, strain BALB/cAnNCrl) were immunized intramuscularly on the right hind leg. BALB/cJ mice (Jackson Laboratories) were used in this study only for DNA sequencing where indicated.

Tumor challenge: For the prophylactic vaccine tumor model, mice were vaccinated on day 0 and 7, and challenged on day 14. For testing the long-term protection of vaccine, mice were challenged on day 80. For the therapeutic vaccine tumor model, mice were inoculated with tumor cells subcutaneously on day 0, and then vaccinated with indicated vaccine on days 5 and 12. Tumor growth was monitored three times a week and tumor sizes were calculated by equation: Tumor volume=length×width²/2. Animals were euthanized when the tumor sizes reached 1 cm in diameter or when animals developed an ulceration. For the experimental lung metastasis tumor model, animals were injected intravenously via tail vein with tumor cells on day 0, then were left untreated or treated with intramuscular injection with the indicated vaccines on day 2 and 9 for the A5 vaccine studies or day 1 and 8 for the RragcL385P, Tmem5S71N or EML5G44R peptide screening studies. Lungs were excised and stained with Bouin's solution (Sigma Catalog: HT10132) on day 18 for mice injected with CT26 cells and on day 16 for mice injected with 4T1 cells. Tumor nodules were counted manually and lung weights were measured.

Acute toxicity studies: 8-week-old female CD-1 mice were either untreated or injected with CPQ/A5 on days 0 and 7 intramuscularly, with doses of 0.5 μg A5 peptide, 2 μg CoPoP, 2 μg PHAD and 2 μg QS-21 per mouse. On day 14, anticoagulated blood and serum were collected for standard complete blood cell count and serum panel, 15 μL of blood was assessed by Heska Element HT5 Hematology Analyzer for complete blood cell count within 4 hours of blood collection. Serum was assessed by the Heska Element DC Chemistry Analyzer. Organs (heart, liver, spleen, lung, kidney) were fixed in formalin, stored in 70% ethanol and subject to hematoxylin and eosin (H&E) staining and imaging.

IFN-γ ELISA: 2.5×10⁵ splenocytes were seeded in a 96 wells plate and stimulated with 10 μg/mL antigens for 72 hr. 50 μL of supernatant was collected from each well and subjected to Interferon gamma ELISA (Thermofisher; catalog: BMS606TEN) according to manufacturer protocol.

Antibody Staining

For tetramer staining, immunized mice were analyzed for the percentages of tumor Ag-specific CD8⁺ T cells by a tetramer staining assay. H-2L^{d}-restricted AH1 (SPSYVYHQF) peptide was complexed with MHC-I (H-2L^{d}) and conjugated with PE (the NIH Tetramer core facility). 60 μL of blood incubated with the AH1 tetramer for 1 hr at 4° C. (100× dilution), then incubated with CD8a, MHC-II (IA/IE), B220, CD4, CD44 and CD62L antibodies for 30 min at 4° C. (1000× dilution). Red blood cells were lysed by cell lysis buffer for 5 mins then cells were centrifuged at 500×g for another 5 min. The cell pellets were washed twice for flow cytometry analysis. For tetramer staining of splenocytes, 1×10⁶ cells were incubated with tetramer and antibodies in the same condition as blood, and then washed twice for flow cytometry analysis. Flow cytometry studies were carried out using a BD LSRFortessam X-20 cytometer. Flowjo (version 10) software was used for data analysis.

For Intracellular staining: 100 μL of 1×10⁶ splenocytes were seeded in a flat bottom 96 wells plate and stimulated with 10 μg/mL antigen for 15-18 hours in the cell culture incubator. Then Brefeldin A was added to the plates with a dilution of 1000× for another 5 hours. Cells were transferred to a round bottom 96 wells plate and centrifuged at 1350 rpm for 3 mins, the cell pellets were washed twice and stained with 500× live/dead fixable dye, 200× diluted APC anti-mouse CD8, 200× diluted FITC anti-mouse CD4, 200× diluted PE/Cy7 anti-mouse CD62L and 200× diluted PerCP/Cy5.5 anti-mouse for 25 min at 4° C. with shaking. Cells were washed twice and fixed and permeabilized by fixation/permeabilization buffer for 20 min at 4° C. Cells were washed twice with perm wash buffer and stained with 200× diluted pacific blue anti-mouse IFN-γ and 200× diluted PE anti-mouse TNF-α for 30 min on ice. Cells were washed twice by perm/wash buffer for flow cytometry.

For cell recruitment studies, CD-1 mice were either untreated or injected intramuscularly with CPQ/A5 or CP/A5. 48 hr later, mice were euthanized and lymph nodes were collected for cell extraction. Cells were fixed with 4% and washed, then stained with combination antibodies against Ly6C, CD11b, Ly6G, CD11c, CD3, I-A/I-E and F4/80 for 1 hr on ice and cells were identified.

For immunofluorescence microscopy, beads were coated by shaking liposomes (containing 320 μg/mL PoP or CoPoP in addition to other components including fluorescent A5) with 25 mg/mL beads (Spherotech Silica Particles, 1.5-1.9 μm; catalog: SIP-15-10) for 10 min at 2000 rpm, followed shaking at 1200 rpm for 45 min. Free liposomes (in the supernatant) were removed by centrifugation at 1200 rcf for 2 min, and beads were washed twice with PBS in this manner. Glass coverslips were treated with 1% of Alcian blue for 10 min at 37° C. in the incubator, followed by 3 washes with PBS. 5×10⁵ BMDCs were seeded on Alcian blue-treated glass coverslips for 30 min, then incubated with liposome-coated silica beads or uncoated silica beads for 3 hr. Cells were washed with PBS 3 times, then fixed with 4% Paraformaldehyde (VWR; catalog: 30525-89-4) for 20 min at 4° C. Slides were washed with PBS 3 times, followed by incubation with 5% BSA with 0.1% Triton X-100 (PBST) for 30 min at room temperature. Cells were incubated with anti-mouse H2Ld (1:500 dilution, Invitrogen, PIMA170109) at 4° C. overnight, and washed with 5% BSA in PBST 3 times, followed by Alexa Flour 555 anti-mouse secondary (1:1000 dilutions, Invitrogen; catalog: A21137) for 30 min at room temperature. The slides were wash with PBST for 3 times, and stained with anti-mouse LAMP1 (1:500 dilution, Invitrogen catalog 50-128-11) for 30 min at room temperature. After incubation, the slides were wash with PBST for 3 times, then stained with Alexa Flour 647 chicken anti-rat IgG (1:1000 dilutions, Invitrogen cat #A21137A21472) for 30 min at room temperature, then wash with PBST for 3 time. Slides stained with 4′,6-diamidino-2-phenylindole (DAPI) in anti-fade mounting medium (Vectashield, catalog: H-1200). Images were acquired on a Zeiss LSM 710 Confocal Microscope.

For Cytotoxic T lymphocyte (CTL) cytotoxicity assay, isolated splenocytes were cultured in the cell culture medium and stimulated by mouse IL-2 (Pepro tech; catalog: 212-12; 10 IU/mL) and antigens (10 μg/mL) for 5 days to use as the effector cells. 5000 CT26 cells were seeded in a 96 wells plate and pulsed with 10 μg/mL antigens for 1 hour, then splenocytes were added to the plate at different E:T ratios for 5 hours. The cytotoxicity of splenocytes on tumor cells was assessed by lactate dehydrogenase (LDH) release using Non-Radioactive Cytotoxicity Assay Kit (Promega; catalog: G1780) according to manufacturer instructions.

DNA Sequencing of RragcL385P in CT26 and 4T1 Cells

DNA was extracted using DNeasy Blood & Tissue kit (QIAGEN, catalog number: 69504) and PCR-amplified using forward primer TCACTGTTCACGTCTGTCCT (SEQ ID NO:46) and reverse primer ACTGAGTTCTGAGGTCTCT (SEQ ID NO:47). 1.5% agarose gels were used to purify DNA, and the DNA bands were cut and extracted using QIAquick Gel Extraction Kit (QIAGEN, catalog number: 28706). The quality and concentration of the isolated PCR products were measured using NanoDrop One (ThermoFisher). The purified DNA was sequenced by the Sanger sequencing method at the DNA Sequencing Core, Baylor College of Medicine, Houston, TX. Data was analyzed by Snapgene.

Statistical Analysis

Data were analyzed by one- or two-way analysis of variance (ANOVA), followed by Bonferroni post hoc test for comparison of multiple groups or analyzed by student t test for comparison of two groups or analyzed by log-rank test for comparison of survival with Prism 8 (GraphPad Software). P values less than 0.05 were considered statistically significant. Values are reported as means±SD with the indicated sample size.

TABLE 1
Peptides used in each figure. Refer to next table for peptide identity.

| | Peptide | | Tumor |
|---|---|---|---|
| FIG. | name | Vaccine dosing | challenge |
| FIG. 1B, D | PEP1, 6 | — | — |
| FIG. 1C, E, F, H | PEP1 | — | — |
| FIG. 1G, I | PEP13 | — | — |
| FIG. 2A | PEP13 | — | — |
| FIG. 2B, C, D | PEP1 | — | — |
| FIG. 3 | PEP1 | 500 ng, day 0 & 7 | — |
| FIG. 4 | PEP1 | 500 ng, day 0 & 7 | |
| FIG. 5A, B, C | PEP1 | 0, 20, 100, 200, | 1 × 10⁵ CT26, |
| | | 500, 750, 1000 ng, | s.c., day 14 |
| | | day & 7 | |
| FIG. 5D, E, F | PEP1 | 500 ng, day 0 & 7 | 1 × 10⁵ CT26, |
| | | | s.c., day 14 |
| FIG. 5G | PEP1 | 500 ng, day 0 & 7 | 1 × 10⁵ CMS4, |
| | | | s.c., day 14 |
| FIG. 5H | PEP1 | 500 ng, day 0 & 7 | 1 × 10⁵ 4T07, |
| | | | mammary fat |
| | | | pad, day 14 |
| FIG. 5I, J | PEP1 | 500 ng, day 0 & 7 | 1 × 10⁵ CT26, |
| | | | s.c., day 80 |
| FIG. 6 | PEP1 | 500 ng or 1000 ng, | — |
| | | day 0 & 7 | |
| FIG. 7 | PEP1 | 500 ng, day 0 & 7 | |
| FIG. 8A, B, C, D, E | PEP12 | 500 ng, day 5 & 12; | 3 × 10⁴ CT26, |
| | | | s.c., day 0 |
| FIG. 8F, G, H | PEP1 | 500 ng, day 2 & 9; | 1 × 10⁵ CT26, |
| | | | i.v., day 0 |
| FIG. 9B | PEP1 | 500 ng, day 0 & 7 | — |
| FIG. 9C, D, E, F | PEP13 | — | — |
| FIG. 11 | PEP13 | — | — |
| FIG. 13 | PEP13 | — | — |
| FIG. 14A | PEP1-5, 12 | — | — |
| FIG. 14B, C | PEP5-11 | 500 ng, day 0 & 7 | — |
| FIG. 15 | PEP5-11 | 500 ng, day 0 & 7 | — |

TABLE 2
AH1 related peptides

| | SEQ ID | | HPLC | Theoretical | Observed |
|---|---|---|---|---|---|
| Name | NO | Sequence | purity | MW | MW |
| PEP1 | 48 | HHHHHHSPSYAYHQFERR | 97.7 | 2363.48 | 2363.75 |
| PEP2 | 49 | HHHHHHSPSYAYHQFEEE | 97.8 | 2309.34 | 2309.6 |
| PEP3 | 50 | HHHHHHSPSYAYHQFRRR | 99.1 | 2390.56 | 2391 |
| PEP4 | 51 | HHHHHHSPSYAYHQF | 95.1 | 1922 | 1922.6 |
| PEP5 | 52 | SPSYAYHQF | 99.3 | 1099.16 | 1099 |
| PEP6 | 53 | SPSYAYHQFERR | 91.2 | 1540.64 | 1540.5 |
| PEP7 | 54 | HSPSYAYHQFERR | 93.4 | 1677.78 | 1677.6 |
| PEP8 | 55 | HHSPSYAYHQFERR | 94.8 | 1814.92 | 1814.8 |
| PEP9 | 56 | HHHSPSYAYHQFERR | 92.2 | 1952.06 | 1952 |
| PEP10 | 57 | HHHHSPSYAYHQFERR | 90.2 | 2089.2 | 2089.2 |
| PEP11 | 58 | HHHHHSPSYAYHQFERR | 96.3 | 2226.34 | 2226 |
| PEP12 | 59 | HHHSPSYVYHQF | 85.6 | 1538.63 | 1538.7 |
| PEP13 | 60 | HHHHHHSPSYAYHQFERR-KHiLyte488 | 93 | 2847.1 | 2848.8 |
| PEP14 | 61 | HHSPSYAYHQFERR-KHiLyte488 | 92 | 2298.5 | 2299.8 |

TABLE 3
CT26 peptides

| | | | SEQ | | | NetMHC | Experi- | | Theo- | Ob- |
|---|---|---|---|---|---|---|---|---|---|---|
| | | | ID | | MHC | per- | mental | HPLC | retical | served |
| Name | Gene | Mut | NO | Sequence | allele | centile | rank | purity | MW | MW |
| CT1 | E2f8 | I522T | 62 | H6-SGPSYATYL- | H-2D^{d} | 0.02 | 62 | 86.6 | 2222.35 | 2222.25 |
| | | | | ERR | | | | | | |
| CT2 | Ints13 | Y529C | 63 | H6-KGPKRDEQC- | H-2D^{d} | 0.025 | 65 | 97.7 | 2324.52 | 2324.7 |
| | | | | ERR | | | | | | |
| CT3 | Aen | A111P | 64 | H6-PGPIKCVPI- | H-2D^{d} | 0.04 | 22 | 98.2 | 2187.51 | 2187.5 |
| | | | | ERR | | | | | | |
| CT4 | Fubp1 | L371F | 65 | H6-GPPGGFQEF- | H-2D^{d} | 0.06 | 63 | 86.5 | 2199.32 | 2199.5 |
| | | | | ERR | | | | | | |
| CT5 | Chd8 | L1008F | 66 | H6-QFPSESEFF- | H-2D^{d} | 0.1 | 18 | 90 | 2381.5 | 2381.75 |
| | | | | ERR | | | | | | |
| CT6 | Trpm7 | A986T | 67 | H6-TGPYVMMIG- | H-2D^{d} | 0.125 | 84 | 85.9 | 2232.52 | 2232.75 |
| | | | | ERR | | | | | | |
| CT7 | Haus6 | A821T | 68 | H6-SYETLKKSL- | H-2K^{d} | 0.01 | 94 | 97.3 | 2332.55 | 2332.6 |
| | | | | ERR | | | | | | |
| CT8 | Hdac2 | P228S | 69 | H6-KYYAVNFSM- | H-2K^{d} | 0.03 | 88 | 76.4 | 2386.63 | 2387.25 |
| | | | | ERR | | | | | | |
| CT9 | Tax1bp1 | H107Y | 70 | H6-CYVTYKGEI- | H-2K^{d} | 0.06 | 89 | 94.7 | 2339.57 | 2339.7 |
| | | | | ERR | | | | | | |
| CT10 | Slc20a1 | T425I | 71 | H6-SYTSYIMAI- | H-2K^{d} | 0.125 | 69 | 89.4 | 2312.54 | 2312.8 |
| | | | | ERR | | | | | | |
| CT11 | Sel1l | A299T | 72 | H6-RYWTGIGVL- | H-2K^{d} | 0.125 | 92 | 89.7 | 2328.57 | 2329.25 |
| | | | | ERR | | | | | | |
| CT12 | Glud1 | V546I | 73 | H6-AYVNAIEKI- | H-2K^{d} | 0.15 | 93 | 87.3 | 2284.51 | 2284.75 |
| | | | | ERR | | | | | | |
| CT13 | Cone1 | D259A | 74 | H6-AYVNATGEV- | H-2K^{d} | 0.15 | 95 | 87.3 | 2187.31 | 2187.75 |
| | | | | ERR | | | | | | |
| CT14 | Phf3 | G1814E | 75 | H6-FPPQNMFEF- | H-2K^{d} | 0.01 | 98 | 83.4 | 2420.64 | 2421 |
| | | | | ERR | | | | | | |
| CT15 | Zeb1 | L659F | 76 | H6-EPQVEPLDF- | H-2L^{d} | 0.04 | 81 | 99.5 | 2337.49 | 2337.49 |
| | | | | ERR | | | | | | |
| CT16 | Csnk2b | G129D | 77 | H6-IPDEAMVKL- | H-2L^{d} | 0.07 | 33 | 96.5 | 2279.56 | 2279.75 |
| | | | | ERR | | | | | | |
| CT17 | Plaa | G54R | 78 | H6-SPNRRFTEM- | H-2L^{d} | 0.175 | 58 | 80.7 | 2401.6 | 2402.1 |
| | | | | ERR | | | | | | |
| CT18 | Fbln5 | D415N | 79 | H6-RPIKGPRNI- | H-2L^{d} | 0.2 | 47 | 93.3 | 2314.59 | 2314.8 |
| | | | | ERR | | | | | | |
| CT19 | Agl | L1529F | 80 | H6-VVLETFYDL- | H-2L^{d} | 0.25 | 56 | 81.5 | 2362.58 | 2362.75 |
| | | | | ERR | | | | | | |
| CT20 | Ubqln1 | A456V | 81 | H6-NPRAMQVLL- | H-2L^{d} | 0.4 | 38 | 85.1 | 2305.6 | 2305.5 |
| | | | | ERR | | | | | | |
| CT21 | Ttc15 | A359P | 82 | H6-DPFATPLSM- | H-2L^{d} | 0.2 | 83 | 90.9 | 2242.45 | 2242.5 |
| | | | | ERR | | | | | | |
| CT22 | Ralgapa2 | A1523P | 83 | H6-EPQIAMDDM- | H-2L^{d} | 0.25 | 74 | 92.7 | 2313.51 | 2313.5 |
| | | | | ERR | | | | | | |
| CT23 | Anks6 | H194Y | 84 | H6-GYEAVVRLL- | H-2L^{d} | 0.6 | 77 | 87.8 | 2283.53 | 2283.75 |
| | | | | ERR | | | | | | |
| CT24 | Mtor | V971M | 85 | H6-HHTMMVQAI- | H-2K^{d} | 0.5 | 86 | 93 | 2331.62 | 2331.6 |
| | | | | ERR | | | | | | |
| CT25 | Eml5 | D1349A | 86 | H6-HYLNDGDAI- | H-2K^{d} | 0.3 | 80 | 95.9 | 2281.38 | 2281.5 |
| | | | | ERR | | | | | | |
| CT26 | Pdgfra | V103I | 87 | H6-LFVTVLEVI- | H-2K^{d} | 3.3 | 91 | 88.5 | 2296.61 | 2296.75 |
| | | | | ERR | | | | | | |
| CT27 | Ptpn13 | S715P | 88 | H6-PYFRLEHYL- | H-2K^{d} | 0.9 | 87 | 97.6 | 2501.74 | 2502 |
| | | | | ERR | | | | | | |
| CT28 | Slc41a2 | G546D | 89 | H6-PYLTALDDL- | H-2K^{d} | 1.9 | 82 | 92.9 | 2284.47 | 2284.75 |
| | | | | ERR | | | | | | |
| CT29 | Noc31 | H231Y | 90 | H6-SYIKKLKEL- | H-2K^{d} | 1.6 | 90 | 96.9 | 2385.7 | 2385.8 |
| | | | | ERR | | | | | | |
| CT30 | Vps26b | G121E | 91 | H6-SYTEQNVKL- | H-2K^{d} | 0.6 | 85 | 99.2 | 2345.51 | 2345.7 |
| | | | | ERR | | | | | | |
| CT31 | Tarsl2 | E353K | 92 | H6-TYWKGNPEM- | H-2K^{d} | 4.4 | 97 | 93.2 | 2389.59 | 2389.4 |
| | | | | ERR | | | | | | |
| CT32 | Rwdd2b | S61N | 93 | H6-VYFTINVNL- | H-2K^{d} | 0.7 | 43 | 97 | 2346.58 | 2347 |
| | | | | ERR | | | | | | |
| CT33 | Dhx35 | T646I | 94 | H6-YYMRDVIAI- | H-2K^{d} | 0.1 | 96 | 93.3 | 2407.69 | 2408.4 |
| | | | | ERR | | | | | | |
| CT34 | Pigo | E293K | 95 | H6-LFLKSPTAL- | H-2K^{d} | 0.3 | 99 | 95.7 | 2253.54 | 2253.9 |
| | | | | ERR | | | | | | |
| CT35 | Qsox1 | G268R | 96 | H6-SYLRRLPGL- | H-2K^{d} | 0,5 | 100 | 98.6 | 2338.61 | 2338.8 |
| | | | | ERR | | | | | | |
| CT36* | Fam120a | Q652K | 97 | H6-KTPEL VEAL- | H-2D^{d} | 4.5 | 51 | 96.2 | 2263.49 | 2263.8 |
| | | | | ERR | | | | | | |
| CT37* | Rragc | L385P | 98 | H6-SPKALAHNG- | H-2L^{d} | 2.5 | 1 | 98.6 | 2158.32 | 2158.5 |
| | | | | ERR | | | | | | |
| CT38* | Ppplr12c | S348F | 99 | H6-SSKHRRSFV- | H-2K^{d} | 4.9 | 45 | 97.6 | 2367.57 | 2367.9 |
| | | | | ERR | | | | | | |
| CT39* | Ppp6r1 | A368T | 100 | H6-KLLASALST- | H-2K^{d} | 12.3 | 5 | 99.4 | 2281.51 | 2281.8 |
| | | | | ERR | | | | | | |
| CT40* | Ppp6r1 | D309N | 101 | H6-NALSSMGAL- | H-2K^{d} | 2 | 6 | 93.1 | 2127.32 | 2127.75 |
| | | | | ERR | | | | | | |
| CT41 | Man2b2 | P285S | 102 | H6-RTPHVLWSW- | H-2D^{d} | 0.125 | 40 | 98.2 | 2445.68 | 2445.9 |
| | | | | ERR | | | | | | |
| CT42 | Gpld1 | R829W | 103 | H6- | H-2D^{d} | 0.15 | 28 | 88.1 | 2224.42 | 2225 |
| | | | | WGAWLSGAL- | | | | | | |
| | | | | ERR | | | | | | |
| CT43 | Phf3 | G1814E | 104 | H6-FPPQNMFEF- | H-2D^{d} | 0.175 | 15 | 77.4 | 2420.64 | 2421.25 |
| | | | | ERR | | | | | | |
| CT44 | Pola1 | C501Y | 105 | H6-KGPYWLEVK- | H-2D^{d} | 0.175 | 44 | 95.7 | 2383.64 | 2384.1 |
| | | | | ERR | | | | | | |
| CT45 | Fbx16 | Q187R | 106 | H6-LVPNRFSRL- | H-2D^{d} | 0.2 | 13 | 98.8 | 2365.63 | 2366.1 |
| | | | | ERR | | | | | | |
| CT46 | Ddr1 | T490P | 107 | H6-RGPPPHSAP- | H-2D^{d} | 0.3 | 72 | 93.6 | 2179.34 | 2179.8 |
| | | | | ERR | | | | | | |
| CT47 | Ddr1 | T490P | 108 | H6-GPPPHSAPC- | H-2D^{d} | 0.3 | 71 | 95.3 | 2126.3 | 2126.7 |
| | | | | ERR | | | | | | |
| CT48 | Col8a1 | G514E | 109 | H6-SGPIGPPEI- | H-2D^{d} | 0.4 | 70 | 86 | 2130.3 | 2130.75 |
| | | | | ERR | | | | | | |
| CT49 | Zfp64 | A641P | 110 | H6-TPPSIFSTQ- | H-2D^{d} | 0.4 | 73 | 82.6 | 2241.4 | 2241.6 |
| | | | | ERR | | | | | | |
| CT50 | Aifm1 | I564N | 111 | H6-YGKGVNFYL- | H-2D^{d} | 0.5 | 68 | 96.3 | 2324.53 | 2324.7 |
| | | | | ERR | | | | | | |
| CT51 | Scaf8 | P993L | 112 | H6-GRPSIDNVL- | H-2D^{d} | 0.5 | 37 | 99.1 | 2234.41 | 2234.7 |
| | | | | ERR | | | | | | |
| CT52 | Rnps1 | P246T | 113 | H6-PTPRRFSPP- | H-2D^{d} | 0.5 | 24 | 96.6 | 2318.53 | 2319 |
| | | | | ERR | | | | | | |
| CT53 | Rab3ip | C373R | 114 | H6-RGGPKKCAL- | H-2D^{d} | 0.5 | 32 | 75.2 | 2193.47 | 2193.9 |
| | | | | ERR | | | | | | |
| CT54 | Pcfl1 | G900S | 115 | H6-EGPHGQPVS- | H-2D^{d} | 0.5 | 57 | 98.8 | 2171.27 | 2171.4 |
| | | | | ERR | | | | | | |
| CT55 | Hdac5 | Y248H | 116 | H6-TPPSHKLPL- | H-2D^{d} | 0.5 | 23 | 95.4 | 2253.5 | 2253.6 |
| | | | | ERR | | | | | | |
| CT56 | Syncrip | G480E | 117 | H6-RGEYEDPYY- | H-2D^{d} | 0.6 | 35 | 99.2 | 2455.54 | 2455.8 |
| | | | | ERR | | | | | | |
| CT57 | Fbln5 | D415N | 118 | H6-KGPRNIQLD- | H-2D^{d} | 0.6 | 48 | 97.1 | 2304.51 | 2304.9 |
| | | | | ERR | | | | | | |
| CT58 | Xrn2 | S485F | 119 | H6-GFPSPLGGI- | H-2D^{d} | 0.7 | 42 | 93.7 | 2108.3 | 2108.75 |
| | | | | ERR | | | | | | |
| CT59 | Pola2 | L314R | 120 | H6-TGRRRTATK- | H-2D^{d} | 0.7 | 30 | 94.9 | 2310.52 | 2310.6 |
| | | | | ERR | | | | | | |
| CT60 | Plaa | G54R | 121 | H6-DSPNRRFTE- | H-2D^{d} | 0.8 | 20 | 99 | 2385.49 | 2385.9 |
| | | | | ERR | | | | | | |
| CT61 | Impdh1 | Y339C | 122 | H6-CQIAMVHYI- | H-2K^{d} | 0.175 | 10 | 96.9 | 2341.65 | 2342.1 |
| | | | | ERR | | | | | | |
| CT62 | Mast2 | P202SS | 123 | H6-SFQPTADEL- | H-2K^{d} | 0.3 | 11 | 93.8 | 2271.38 | 2271.9 |
| | | | | ERR | | | | | | |
| CT63 | E2f8 | I522T | 124 | H6-SYATYLQPA- | H-2K^{d} | 0.3 | 19 | 96.7 | 2277.43 | 2278 |
| | | | | ERR | | | | | | |
| CT64 | Cep295 | S136N | 125 | H6-LYLANLRHM- | H-2K^{d} | 0.3 | 14 | 91.3 | 2394.7 | 2395.5 |
| | | | | ERR | | | | | | |
| CT65 | Suv39h2 | D376Y | 126 | H6-YYESDEFTV- | H-2K^{d} | 0.3 | 17 | 78 | 2416.5 | 2417 |
| | | | | ERR | | | | | | |
| CT66 | Tnfaip1 | D50N | 127 | H6- | H-2K^{d} | 0.4 | 61 | 98.8 | 2365.68 | 2366.4 |
| | | | | RHNTMLKAM- | | | | | | |
| | | | | ERR | | | | | | |
| CT67 | Slc20a1 | T425I | 128 | H6-SYIMAICGM- | H-2K^{d} | 0.4 | 36 | 75.9 | 2252.58 | 2252.75 |
| | | | | ERR | | | | | | |
| CT68 | Brcc3 | R186K | 129 | H6-EYEKIEIPI- | H-2K^{d} | 0.4 | 66 | 98.2 | 2397.62 | 2397.9 |
| | | | | ERR | | | | | | |
| CT69 | Tomm70a | K129Q | 130 | H6-KYFQAGKYE- | H-2K^{d} | 0.4 | 21 | 86.7 | 2397.59 | 2398.2 |
| | | | | ERR | | | | | | |
| CT70 | Elmo2 | A239V | 131 | H6-TY AIALINV- | H-2K^{d} | 0.4 | 29 | 87.3 | 2241.49 | 2241.75 |
| | | | | ERR | | | | | | |
| CT71 | Eml5 | G44R | 132 | H6-VYFVAGVRV- | H-2K^{d} | 0.5 | 3 | 98.3 | 2273.53 | 2274 |
| | | | | ERR | | | | | | |
| CT72 | Fbxl6 | Q187R | 133 | H6-SRLQSLTLI- | H-2K^{d} | 0.5 | 7 | 96.7 | 2294.55 | 2294.7 |
| | | | | ERR | | | | | | |
| CT73 | Cwf1912 | L610F | 134 | H6-FFMKMASKF- | H-2K^{d} | 0.5 | 78 | 96 | 2400.76 | 2401.2 |
| | | | | ERR | | | | | | |
| CT74 | Qsox1 | G268R | 135 | H6-FYTSYLRRL- | H-2K^{d} | 0.5 | 76 | 96.9 | 2482.74 | 2483.1 |
| | | | | ERR | | | | | | |
| CT75 | E2f8 | I522T | 136 | H6-TYLQPAQAQ- | H-2K^{d} | 0.6 | 12 | 96.3 | 2283.44 | 2284 |
| | | | | ERR | | | | | | |
| CT76 | Hnrnpl | I203R | 137 | H6-IYSRTTDVL- | H-2K^{d} | 0.5 | 64 | 97.9 | 2331.53 | 2331.9 |
| | | | | ERR | | | | | | |
| CT77 | Foxp4 | S573N | 138 | H6-SYQAALAEN- | H-2K^{d} | 0.6 | 39 | 94.7 | 2230.33 | 2230.75 |
| | | | | ERR | | | | | | |
| CT78 | Kdelr1 | L132M | 139 | H6-IYLESVAIM- | H-2K^{d} | 0.7 | 50 | 71.4 | 2302.59 | 2302.75 |
| | | | | ERR | | | | | | |
| CT79 | Sp3 | Q434H | 140 | H6-GHVAAGGAL- | H-2K^{d} | 0.6 | 46 | 96 | 2016.16 | 2016.5 |
| | | | | ERR | | | | | | |
| CT80 | Snd1 | G169S | 141 | H6-SEGNSSHTI- | H-2K^{d} | 0.8 | 31 | 98.4 | 2195.25 | 2195.4 |
| | | | | ERR | | | | | | |
| CT81 | Cpd | M945L | 142 | H6-LNYPHITNL- | H-2L^{d} | 0.4 | 54 | 98.5 | 2348.56 | 2349.3 |
| | | | | ERR | | | | | | |
| CT82 | Phf3 | G1814E | 143 | H6-PPQNMFEFP- | H-2L^{d} | 0.6 | 67 | 99.8 | 2370.58 | 2370.75 |
| | | | | ERR | | | | | | |
| CT83 | Chd8 | L1008F | 144 | H6-FPSESEFFK- | H-2L^{d} | 0.6 | 26 | 75.7 | 2381.54 | 2382 |
| | | | | ERR | | | | | | |
| CT84 | Smc2 | R1132Q | 145 | H6-IGQMLQTHF- | H-2L^{d} | 0.7 | 49 | 96.9 | 2338.59 | 2338.8 |
| | | | | ERR | | | | | | |
| CT85 | Ankrd13b | A429T | 146 | H6-IPIFHILNT- | H-2L^{d} | 0.7 | 8 | 97.2 | 2331.61 | 2332.2 |
| | | | | ERR | | | | | | |
| CT86 | Steap1 | G165D | 147 | H6-MLARKQFDL- | H-2L^{d} | 0.7 | 60 | 97.1 | 2385.69 | 2385.9 |
| | | | | ERR | | | | | | |
| CT87 | Vps26b | G121E | 148 | H6-KPYESYTEQ- | H-2L^{d} | 0.7 | 75 | 90.7 | 2408.52 | 2409 |
| | | | | ERR | | | | | | |
| CT88 | Mkrn2 | S34F | 149 | H6-KPFTICKYY- | H-2L^{d} | 0.8 | 34 | 99.3 | 2426.73 | 2427 |
| | | | | ERR | | | | | | |
| CT89 | Fubp1 | L371F | 150 | H6-PGGFQEFNF- | H-2L^{d} | 0.8 | 25 | 94.2 | 2306.43 | 2306.5 |
| | | | | ERR | | | | | | |
| CT90 | Pdgfrb | N718T | 151 | H6-TALPVGFSL- | H-2L^{d} | 0.8 | 79 | 98.4 | 2168.39 | 2168.75 |
| | | | | ERR | | | | | | |
| CT91 | Pola1 | C501Y | 152 | H6-RKIKGPYWL- | H-2L^{d} | 0.9 | 52 | 98.2 | 2424.74 | 2425.2 |
| | | | | ERR | | | | | | |
| CT92 | Sel1l | A299T | 153 | H6-LGYRYWTGI- | H-2L^{d} | 0.9 | 41 | 97.6 | 2392.61 | 2392.9 |
| | | | | ERR | | | | | | |
| CT93 | Gyk | P107LL | 154 | H6-LLYNAVVWL- | H-2L^{d} | 0.9 | 27 | 96.8 | 2354.65 | 2354.75 |
| | | | | ERR | | | | | | |
| CT94 | Xrn2 | D763N | 155 | H6-DPQFAENYV- | H-2L^{d} | 0.9 | 55 | 77.1 | 2346.45 | 2346.5 |
| | | | | ERR | | | | | | |
| CT95 | Hdac2 | P228S | 156 | H6-KYYAVNFSM- | H-2L^{d} | 0.9 | 59 | 94.9 | 2386.63 | 2386.8 |
| | | | | ERR | | | | | | |
| CT96 | Tmem5 | S71N | 157 | H6- | H-2L^{d} | 0.9 | 2 | 70.7 | 2326.57 | |
| | | | | VVEANWTML- | | | | | | |
| | | | | ERR | | | | | | |
| CT97 | Man2b2 | P285S | 158 | H6-TPHVLWSWG- | H-2L^{d} | 0.9 | 4 | 91.4 | 2346.54 | 2346.9 |
| | | | | ERR | | | | | | |
| CT98 | Nudt19 | L335F | 159 | H6-YVYEIYMTF- | H-2L^{d} | 1 | 9 | 76.6 | 2492.75 | 2493.5 |
| | | | | ERR | | | | | | |
| CT99 | Fubp1 | L371F | 160 | H6-GPPGGFQEF- | H-2L^{d} | 1 | 53 | 98.7 | 2199.32 | 2199.75 |
| | | | | ERR | | | | | | |
| CT100 | Kdelr1 | L132M | 161 | H6-MPQLFMVSK- | H-2L^{d} | 1.1 | 16 | 82.4 | 2344.7 | 2344.8 |
| | | | | ERR | | | | | | |

| | | | | | | |
|---|---|---|---|---|---|---|
| 3his-CT37/RragcL385P | 162 | H3SPKALAHNG | H-2L^{d} | 94.1 | 1305.41 | 1305.6 |
| 3his-CT71/Eml5G44R | 163 | H3VYFVAGVRV | H-2K^{d} | 95.9 | 1420.63 | 1420.5 |
| 3his-CT96/Tmem5S71N | 164 | H3VVEANWTML | H-2L^{d} | 97.2 | 1473.67 | 1473.4 |

*indicates mutation is shared in CT26 and 4T1.

TABLE 4
RENCA peptides screened.

| | | | SEQ | | | NetMHC |
|---|---|---|---|---|---|---|
| | | | ID | | MHC | percentile/ |
| Name | Gene | Mut | NO | Sequence | allele | Rank |
| Renca_1 | Syne2 | L4492R | 165 | 3His-AYATTYRQL | H-2K^{d} | 0.04 |
| Renca_2 | Syne2 | L4492R | 166 | 3His-AYTTQREEL | H-2K^{d} | 0.06 |
| Renca_3 | Mrps9 | K384M | 167 | 3His-EGARRMFTW | H-2D^{d} | 0.01 |
| Renca_4 | Ipo9 | A698V | 168 | 3His-FPAVVQCTL | H-2L^{d} | 0.1 |
| Renca_5 | Fnbp1 | K315T | 169 | 3His-GGTSRGKLW | H-2D^{d} | 0.08 |
| Renca_6 | Tmem189 | V202I | 170 | 3His-GLPYWVTIL | H-2D^{d} | 0.1 |
| Renca_7 | Pank2 | L176I | 171 | 3His-GYFGAVGAI | H-2K^{d} | 0.06 |
| Renca_8 | Yme111 | A539G | 172 | 3His-GYHESGHAI | H-2K^{d} | 0.01 |
| Renca_9 | Fndc3b | E728Q | 173 | 3His-HGPQLECTV | H-2D^{d} | 0.09 |
| Renca_10 | Actb | N280T | 174 | 3His-IHETTFTSI | H-2K^{d} | 0.07 |
| Renca_11 | Scap | F509V | 175 | 3His-IYVLARTRL | H-2K^{d} | 0.04 |
| Renca_12 | Mfsd1 | A339T | 176 | 3His-LYAVATTLV | H-2K^{d} | 0.09 |
| Renca_13 | Rock2 | A145P | 177 | 3His-MPFANSPWV | H-2L^{d} | 0.07 |
| Renca_14 | Pum3 | E500Q | 178 | 3His-SYLQGHTQQ | H-2K^{d} | 0.06 |
| Renca_15 | Slc12a6 | F254V | 179 | 3His-SYVMISRAL | H-2K^{d} | 0.04 |
| Renca_16 | mt-Col | A303T | 180 | 3His-TYFTSATMI | H-2K^{d} | 0.01 |
| Renca_17 | Usp38 | R327G | 181 | 3His-VGPGALAVL | H-2D^{d} | 0.025 |
| Renca_18 | Polr2j | A30T | 182 | 3His-VPNTCLFTI | H-2L^{d} | 0.025 |
| Renca_19 | Vat1 | A165V | 183 | 3His-VPSVQTFLM | H-2L^{d} | 0.1 |
| Renca_20 | Edem3 | E5425D | 184 | 3His-YYLDVGKTL | H-2K^{d} | 0.02 |

TABLE 5
Predicted MHC-I epitopes in E6/E7 oncogenes sequenced from TC-1
cells.

| | SEQ | | MHC | NetMHC | HPLC | Theoretical | Observed |
|---|---|---|---|---|---|---|---|
| Position | ID NO | Sequence* | allele | percentile | purity (%) | M.W. | M.W. |
| E7₄₉₋₅₇ | 185 | H3-RAHYNIVTF | H-2D^{b} | 0.0101 | 98 | 1531.7 | 1531.6 |
| E7₇₁₋₇₉ | 186 | H3-STHVDIRTL | H-2D^{b} | 0.3098 | 97.7 | 1452.6 | 1452.4 |
| E7₆₋₁₄ | 187 | H3-TLHEYMLDL | H-2D^{b} | 0.9233 | 92.9 | 1545.7 | 1545.9 |
| E6₆₁₋₆₉ | 188 | H3-YRDGNPYAV | H-2D^{b} | 0.6144 | 98.4 | 1465.5 | 1465.2 |
| E6₆₂₋₇₀ | 189 | H3-FAFRDLCIV | H-2D^{b} | 0.7017 | 91.0 | 1494.7 | 1494.6 |
| E6₈₂₋₉₀ | 190 | H3-KCLKFYSKI | H-2K^{b} | 0.4193 | 95.3 | 1540.8 | 1540.8 |

*H3 represents three histidine residues on the N terminus. M.W. represents molecular weight.

TABLE 6
Trp2 peptides

| | SEQ ID | | MHC | NetMHC | HPLC | Observed | Theoretical |
|---|---|---|---|---|---|---|---|
| Name | NO | Sequence | allele | percentile | purity | MW | MW |
| Trp2_1 | 191 | HHHXVYDFFVWL | H-2K^{b} | | Crude | | |
| Trp2_2 | 192 | HHHSXYDFFVWL | H-2K^{b} | | Crude | | |
| Trp2_3 | 193 | HHHSVXDFFVWL | H-2K^{b} | | Crude | | |
| Trp2_4 | 194 | HHHSVYXFFVWL | H-2K^{b} | | Crude | | |
| Trp2_5 | 195 | HHHSVYDXFVWL | H-2K^{b} | | Crude | | |
| Trp2_6 | 196 | HHHSVYDFXVWL | H-2K^{b} | | Crude | | |
| Trp2_7 | 197 | HHHSVYDFFXWL | H-2K^{b} | | Crude | | |
| Trp2_8 | 198 | HHHSVYDFFVXL | H-2K^{b} | | Crude | | |
| Trp2_9 | 199 | HHHSVYDFFVWX | H-2K^{b} | | Crude | | |
| H3-Trp2 | 200 | HHHSVYDFFVWL | H-2K^{b} | 0.7 | 83.5 | 1586.4 | 1586.76 |
| H3-Trp2_8A | 201 | HHHSVYDFFVAL | H-2K^{b} | 0.1 | 99.2 | 1471.4 | 1471.62 |
| H3-Trp2_8R | 202 | HHHSVYDFFVRL | H-2K^{b} | 0.07 | 97.8 | 1556.7 | 1556.73 |
| H3-Trp2_8N | 203 | HHHSVYDFFVNL | H-2K^{b} | 0.04 | 72.4 | 1514.7 | 1514.65 |
| H3-Trp2_8D | 204 | HHHSVYDFFVDL | H-2K^{b} | 0.25 | 98.8 | 1515.3 | 1515.63 |
| H3-Trp2_8C | 205 | HHHSVYDFFVCL | H-2K^{b} | 0.4 | 97.1 | 1503.4 | 1503.69 |
| H3-Trp2_8Q | 206 | HHHSVYDFFVQL | H-2K^{b} | 0.12 | 74.3 | 1528.5 | 1528.68 |
| H3-Trp2_8E | 207 | HHHSVYDFFVEL | H-2K^{b} | 0.2 | 96 | 1529.6 | 1529.66 |
| H3-Trp2_8G | 208 | HHHSVYDFFVGL | H-2K^{b} | 0.07 | 99.1 | 1457.4 | 1457.6 |
| H3-Trp2_8H | 209 | HHHSVYDFFVHL | H-2K^{b} | 0.06 | 96.7 | 1537.5 | 1537.69 |
| H3-Trp2_8I | 210 | HHHSVYDFFVIL | H-2K^{b} | 0.4 | 96 | 1513.5 | 1513.7 |
| H3-Trp2_8L | 211 | HHHSVYDFFVLL | H-2K^{b} | 0.15 | 92 | 1513.8 | 1513.7 |
| H3-Trp2_8K | 212 | HHHSVYDFFVKL | H-2K^{b} | 0.17 | 98.2 | 1528.5 | 1528.72 |
| H3-Trp2_8M | 213 | HHHSVYDFFVML | H-2K^{b} | 0.15 | 96.1 | 1531.8 | 1531.74 |
| H3-Trp2_8F | 214 | HHHSVYDFFVFL | H-2K^{b} | 0.17 | 94.6 | 1547.7 | 1547.72 |
| H3-Trp2_8P | 215 | HHHSVYDFFVPL | H-2K^{b} | 0.06 | 94.7 | 1497.4 | 1497.66 |
| H3-Trp2_8S | 216 | HHHSVYDFFVSL | H-2K^{b} | 0.1 | 89.6 | 1487.4 | 1487.62 |
| H3-Trp2_8T | 217 | HHHSVYDFFVTL | H-2K^{b} | 0.12 | 93.7 | 1501.65 | 1501.65 |
| H3-Trp2_8W | 218 | HHHSVYDFFVWL | H-2K^{b} | 0.7 | 96.7 | 1586.7 | 1586.76 |
| H3-Trp2_8Y | 219 | HHHSVYDFFVYL | H-2K^{b} | 0.09 | 98.5 | 1563.6 | 1563.72 |
| H3-Trp2_8V | 220 | HHHSVYDFFVVL | H-2K^{b} | 0.4 | 98.2 | 1499.4 | 1499.68 |
| Trp2 | 221 | SVYDFFVWL | H-2K^{b} | 0.04 | 96.7 | 1174.6 | 1175.34 |

While the invention has been described through specific embodiments, routine modifications will be apparent to those skilled in the art and such modifications are intended to be within the scope of the present disclosure.

What is claimed is:

1. A method for reducing the growth of the tumor in a subject comprising administering to a subject in need of treatment a composition comprising:
a) liposomes comprising i) a bilayer, wherein the bilayer comprises one or more phospholipids and one or more porphyrin phospholipid conjugates having cobalt coordinated thereto forming cobalt-porphyrin phospholipid conjugate; and ii) a polyhistidine-tagged MHC-I restricted tumor peptide, wherein at least a portion of the amino acid sequence of the MHC-I restricted peptide is exposed to the outside of the liposome; and
b) a pharmaceutical carrier,
wherein administration of the composition results in inhibition of growth of the tumor.

2. A method for generating an immune response against a tumor antigen comprising administering to a subject afflicted with the tumor a composition comprising:
a) liposomes comprising i) a bilayer, wherein the bilayer comprises one or more phospholipids and one or more porphyrin phospholipid conjugates having cobalt coordinated thereto forming cobalt-porphyrin phospholipid conjugate; and ii) a polyhistidine-tagged MHC-I restricted tumor peptide, wherein at least a portion of the amino acid sequence of the MHC-I restricted peptide is exposed to the outside of the liposome; and
b) a pharmaceutical carrier,
wherein administration of the composition results in inhibition of growth of the tumor.

3. The method of claim 2, wherein the immune response is an induction of CD8+ T cells.

4. The method of any one of claims 1-3, wherein the MHC-I binding peptide is 7 to 11 amino acids long excluding the polyhistidine tag.

5. The method of any one of claims 1-3, wherein the MHC-I binding peptide does not significantly bind to MHC-II molecules.

6. The method of any one of claims 1-3, wherein the polyhistidine tag comprises 6 to 10 histidine residues.

7. The method of any one of claims 1-3, wherein the liposome further comprises one or more adjuvants incorporated therein.

8. The method of claim 7, wherein the one or more additional adjuvant is QS21 and/or MPLA.

9. The method of claim 7, wherein the one or more additional adjuvant further comprises MPLA or a synthetic variant thereof.

10. The method of claim 9, wherein the synthetic variant is PHAD, 3D6A-PHAD, 3D-PHAD) or a combination thereof.

11. The method of claim 7, wherein the mass ratio of the MHC-I restricted peptide to the adjuvant is from 1:1 to 10:1.

12. The liposome of claim 11, wherein the MHC-I restricted peptide, the QS21 and PHAD are present in mass ratios of 1:1:1, 2:1:1, 3:1:1, 4:1:1, 5:1:1, 6:1:1, 7:1:1, 8:1:1, 9:1:1, or 10:1:1.

13. The method of any one of claims 1-3, wherein the subject is a human.

14. The method of any one of claims 1-3, wherein the composition is administered multiple times.

15. A vaccine composition comprising:
a) liposomes comprising i) a bilayer, wherein the bilayer comprises one or more phospholipids and one or more porphyrin phospholipid conjugates having cobalt coordinated thereto forming cobalt-porphyrin phospholipid conjugate; and ii) a polyhistidine-tagged amino acid sequence of a MHC-I binding tumor peptide, wherein at least a portion of the polyhistidine tag resides in the hydrophobic portion of the bilayer and one or more histidines of the polyhistidine tag are coordinated to the cobalt in the cobalt-porphyrin phospholipid conjugate, and wherein at least a portion of the MHC-I binding tumor peptide sequence is exposed to the outside of the liposome; and
b) a pharmaceutical carrier.

16. The vaccine composition of claim 15, wherein the polyhistidine-tag comprises 2-6 histidine residues.

17. The vaccine composition of claim 15, wherein the MHC-I restricted peptides does not bind to MHC-II class molecules.

18. The vaccine composition of claim 15, wherein the liposome further comprises one or more additional adjuvants incorporated therein.

19. The vaccine composition of claim 18, wherein the one or more adjuvant is QS21 and/or PHAD.

20. The vaccine composition of claim 18, wherein the one or more additional adjuvant further comprises MPLA or a variant thereof.

21. The vaccine composition of claim 20, wherein the MPLA variant is PHAD, 3D6A-PHAD, or 3D-PHAD.

22. The vaccine composition of claim 19, wherein the mass ratio of the MHC-I restricted peptide to the QS21 or PHAD is from 1:1 to 10:1.

23. The vaccine composition of claim 19, wherein the MHC-I restricted peptide, the QS21 and PHAD are present in mass ratios of 1:1:1, 2:1:1, 3:1:1, 4:1:1, 5:1:1, 6:1:1, 7:1:1, 8:1:1, 9:1:1, or 10:1:1.

24. The vaccine composition of claim 15, wherein the bilayer further comprises a cobalt porphyrin.

25. The vaccine composition of claim 24, wherein the cobalt porphyrin-phospholipid conjugate makes up from 1 to 25 mol % of the monolayer or the bilayer.

26. The vaccine composition of claim 25, wherein the cobalt porphyrin-phospholipid conjugate makes up from about 2% to about 8%, preferably 3-4% mass ratio of the bilayer.

27. The vaccine composition of claim 15, wherein the bilayer further comprises cholesterol with mass ratio of about 15 to 20%.

28. The vaccine composition of claim 15, wherein size of the liposome is 50 nm to 250 nm.

29. A method comprising immunizing mice with a combination of candidate neoepitope peptides and selecting one or more the peptides that bind to MHC-I and stimulate CD8+ T cells in the mice.

30. The method of claim 29, further comprising formulating a composition for use in stimulating CD8+ T cells with one or more of the selected peptides.

31. A method comprising randomization of one or a combination of amino acids in one or more candidate neoepitope peptides, testing peptides comprising the one or more randomized amino acids, and comparing a property of one or more of said peptides to properties of an unrandomized peptide used for randomization to identify one or more peptides with improved properties.

32. The method of claim 31, wherein the one or more improved properties comprises a higher affinity for MHC-1, improved activation of CD8+ T cells, improved anti-cancer activity, or a combination thereof.
